(19) United States
(12) Patent Application Publication
(10) Pub. No.: US 2014/0243239 A1
(43) Pub. Date: Aug. 28, 2014

(54) MULTIPLEXED KINASE INHIBITOR BEADS AND USES THEREOF

(71) Applicant: University of North Carolina at Chapel Hill, Chapel Hill, NC (US)

(72) Inventors: Gary Johnson, Chapel Hill, NC (US); James S. Duncan, Chapel Hill, NC (US); Martin C. Whittle, Chapel Hill, NC (US); Jin Jian, Chapel Hill, NC (US)

(21) Appl. No.: 14/351,362

(22) PCT Filed: Oct. 10, 2012
(86) PCT No.: PCT/US2012/059535

§ 371 Date: Apr. 11, 2014

Related U.S. Application Data
(60) Provisional application No. 61/546,399, filed on Oct. 12, 2011.

(51) Int. Cl. G01N 33/573

(52) U.S. Cl. CPC G01N 33/573
USPC 506/9; 506/15

(57) ABSTRACT

This invention is directed to a multi-analyte column comprising two or more layers with a first solid support having specific binding affinity for kinases and a second solid support having non-specific binding affinity for kinases. Methods are also provided, including methods of detecting low abundance kinases, predicting resistance to chemotherapy, determining cancer prognosis, and improving the effectiveness of a treatment regimen.

### CROSS REFERENCE TO RELATED APPLICATIONS

This application claims the benefit of U.S. Provisional Application No. 61/546,399 filed Oct. 12, 2011, Johnson et al., having Atty. Dkt. No. UNC11001USV, which is hereby incorporated by reference in its entirety.

### STATEMENT REGARDING FEDERALLY SPONSORED RESEARCH OR DEVELOPMENT

This invention was made at least in part with government support under grant numbers CA58223, DK37871 and GM30324 awarded by the National Institutes of Health. The United States Government has certain rights in the invention.

### 1. FIELD OF THE INVENTION

This invention relates generally to the discovery of a multi-analyte column comprising two or more layers with a first solid support having specific binding affinity for kinases and a second solid support having non-specific binding affinity for kinases. Methods are also provided, including methods of detecting low abundance kinases, predicting resistance to chemotherapy, determining cancer prognosis, and improving the effectiveness of a treatment regimen.

### 2. BACKGROUND OF THE INVENTION

### 2.1. Kinases and Phosphatases

The phosphorylation state of proteins in eukaryotic cells is responsible for much of signal transduction and controls essential cellular processes such as metabolism, transcription, cell cycle progression, cytoskeletal rearrangement and cell movement, apoptosis, and differentiation. Manning et al., 2002 Science 298 1912-1934. The reversible phosphorylation is performed by kinases and phosphatases which are either receptor (transmembrane) or cytoplasmic. There are 518 putative protein kinases in the human genome of which 90 are tyrosine kinases (PTKs) and 428 are serine/threonine kinases (PSKs). Shi, 2009 Cell 139 468-484. The putative phosphatases are fewer, 107 tyrosine phosphatases and ˜30 serine/threonine phosphatases. Shi, 2009.

The cellular phosphorylation state, collectively the kinome, has been associated with a variety of disorders including a wide variety of cancers, autoimmune diseases, metabolic disorders, and neurological disorders. Many drugs have been approved or are in the pipeline that target kinases. Examples of approved small molecule kinase inhibitors are imatinib (Gleevac®) an inhibitor of breakpoint cluster region-abelson (BCR-ABL) approved initially for chronic myelogenous leukemia (CML). Another drug, sirolimus (Rapamune®), an inhibitor of mammalian inhibitor of rapamycin (mTOR), was approved initially as an immunosuppressant. Examples of monoclonal antibody kinase inhibitors are trastuzumab (Herceptin®), an inhibitor of ERB-B2 and approved for breast cancer or bevacizumab (Avastin®) an inhibitor of vascular endothelial growth factor (VEGF) approved for colorectal cancer. Table 1 lists a number of approved kinase inhibitor drugs. See, Janne et al., 2009 Nat. Rev. Drug Disc. 8 709-723; Levitzki and Klein, 2010 Mol. Aspects Med. 31, 287-329; and Mellor et al. 2011 Tox. Sci. 120(1) 14-32.

TABLE 1

| | | Intended Kinase | |
|---|---|---|---|
| Drug | Class | Target(s) | Approved Indication |
| Axitinib (Inlyta ®) | Small molecule | VEGFR-1, VEGFR-2, | 2^{nd} line RCC |
| | | and VEGFR-3 | |
| Bevacizumab (Avastin ®) | Monoclonal | VEGF | Colorectal cancer, met BrCA |
| Cetuximab (Erbitux ®) | Monoclonal | ERB-B1 | Head & Neck cancer |
| Crizotinib (Xalkori ®) | Small molecule | EML4-ALK | NSCLC |
| | | rearrangements | |
| Dasatinib (Sprycel ®) | Small molecule | ABL, ARG, KIT, | CML with imatinib resist. |
| | | PDGFRα/β, SRC | and/or intolerance |
| Erlotinib (Tarceva ®) | Small molecule | EGFR | NSCLC and pancreatic |
| | | | carcinoma |
| Everolimus (Afinitor ®) | Small molecule | mTOR | BrCA, pancreatic, RCC |
| Gefitinib (Iressa ®) | Small molecule | EGFR | NSCLC |
| Imatinib (Gleevec ®) | Small molecule | ABL, ARG, PDGFR-α/β, | CML, GIST, B-ALL, CMML, |
| | | KIT | CEL |
| Lapatinib (Tykerb ®) | Small molecule | EFGR (ERB-B1 and 2) | HER2 positive BrCA |
| Nilotinib (Tasigna ®) | Small molecule | ABL, ARG, KIT, | CML with imatinib resist. |
| | | PDGFRα/β | and/or intolerance |
| Panitumumab (Vectibix ®) | Monoclonal | EGFR | Colorectal cancer |
| Pazopanib (Votrient ®) | Small molecule | VEGFR, PDGFRα/β, and | RCC, 2^{nd} line adv STS |
| | | KIT | |
| Pegaptanib (Macugen ®) | RNA aptamer | VEGF | Macular degeneration |
| Ranibizumab (Lucentis ®) | Monoclonal | VEGF-A | Macular degeneration |
| Ruxolitinib (Jakafi ™) | Small molecule | JAK1, JAK2 | Myelofibrosis |
| Sirolimus (Rapamune ®) | Small molecule | mTOR | Transplant rejection |
| Sorafenib (Nexavar ®) | Small molecule | B-RAF, VEGFRs, | RCC, liver carcinoma |
| | | PDGFRα/β, FLT3, KIT | |
| Sunitinib (Sutent ®) | Small molecule | VEGFR, PDGFR, | RCC, GIST |
| | | CSF1R, FLT3, KIT | |
| Temsirolimus (Torisel ®) | Small molecule | mTOR | RCC |
| Trastuzumab (Herceptin ®) | Monoclonal | ERB-B2 | HER2 positive BrCA |
| Vandetanib (Caprelsa ®) | Small molecule | EGFR, VEGFR, TIE2, | Thyroid cancer |
| Vemurafenib (Zelboraf ®) | Small molecule | B-RAF | Melanoma |

Abbreviations:
For kinase targets see Gene Cards (http://www.genecards.org/). For indications B-ALL,
Acute B Lymphoblastic Leukemia;
BrCA, breast cancer;
CEL, chronic eosinophilic leukemia;
CML, chronic myeloid leukemia;
CMML, chronic myelomonocytic leukemia;
GIST, gastrointestinal stromal tumor;
NSCLC, non-small cell lung cancer;
RCC, renal cell carcinoma;
STS, soft tissue sarcoma.

Many of these drugs are approved for use with a companion diagnostic. For example, trastuzumab (Herceptin®) is approved for breast cancer over expressing ERB-B2 and cetuximab (Erbitux®) for patients with wild-type KRAS. Amado et al., 2008, J Clin Oncol 26 (10): 1626-1634; Allegra et al., 2009 J Clin Oncol 27 2091-2096. Another kinase inhibitor approved for use with a diagnostic is crizotinib (Xalkori®) approved with a fluorescent in situ hybridization (FISH) test for ALK rearrangements (Vysis LSI ALK Dual Color, Break Apart Rearrangement Probe; Abbott Molecular, Abbott Park, Ill.). Shah et al., 2011 Lancet Oncol 12 1004-1012; Shaw et al., 2009 J Clin Oncol 27 4247-4253. Vemurafenib (Zelboraf®) is approved for use in patients with BRAF V600E mutation (Cobas 4800 BRAF V600 Mutation Test, Roche Molecular Diagnostics, Pleasanton, Calif.). Chapman et al., 2011 NEJM 364 2507-2516.

### 2.2. Current Methods to Study the Kinome

From the early days of 2-D gel chromatography more than thirty years ago scientists have used mass spectroscopic techniques to analyze proteins and metabolites. These techniques have advanced greatly over the decades. However, most approaches require large sample sizes or knowledge a priori of the kinase structure. Ciaccio et al. recently reported using specific phospho-antibodies in a microwestern array to measure 91 phosphosites on 67 proteins in a cancer cell line after stimulation with EGF. Ciaccio et al., 2010, Nat. Meth. 7(2) 148-157. However, these methods may be biased based on the choice of antibody binding sites and phosphorylation state.

Wissing et al. reported using four consecutive columns containing affinity matrices with structurally different kinase ligands as immobilized capture ligands. Wissing et al. 2007, MCB, 537-547, FIG. 1. After sample loading and washing, the columns were disconnected and proteins released by column specific elution procedures (using the corresponding free inhibitor). Wissing et al. analyzed cell lines and required ˜10⁹ cells. Daub et al. report using five different immobilized kinase inhibitors with distinct, overlapping kinase binding profiles in three consecutive columns (VI16832, bisindoylmaleimide-X, AX14596, SU6668, and purvalanol B. Daub et al., 2008, Mol Cell 31 438-448, FIG. 1B. Bantscheff et al. report contacting a sample simultaneously with seven immobilized inhibitor beads (Kinobeads) and elution with free inhibitor or compound of interest. Bantscheff et al., 2007, Nat Biotech 25(9) 1035-1044, Supplementary Methods, page 2, ˜5 mg of protein; WO 2006/134056, Drewes et al. page 43, 50 mg protein; elution procedure from the beads, page 79.

However, nearly all the work to date has been performed with passaged cells or cell lines rather than actual tissue samples. The changes associated with passaging cells and/or immortalized cell lines create artifacts that reduce their usefulness. Staveren et al., 2009, Biochim. Biophys. Acta Rev. Cancer, 1795 (2) 92-103. Because of the quantity of sample required, the methods described in paragraph 7 above are not suited to analysis of small samples from human tissues and thus not feasible for clinical use on samples from patient biopsies.

### 2.3. Targeted Cancer Therapies

Unfortunately, the function for most of the kinome remains unknown particularly for actual human or animal samples. As Fedorov et al. reported “academic research is largely bias[ed] towards kinases with well-established roles in cellular signaling” and about 50% of the kinome is largely uncharacterized. Fedorov et al., 2010, Nat. Chem. Biol. 6, 166-169. Rationally devising novel kinase inhibitor combination therapies requires detailed knowledge of kinome activity, not simply measuring the effect of an inhibitor on one or a few kinases in a pathway.

Moreover, molecularly targeted cancer therapies can fail when tumor cells circumvent the action of a single inhibitor, facilitating the development of resistance. Acquired or selected mutations can decrease affinity for therapeutic kinase inhibitors, but resistance also develops by alternate kinase activation bypassing the action of a highly specific inhibitor. Chandarlapaty et al., 2011 Cancer Cell 19, 58-71; Hochgräfe et al., 2010 Cancer Research 70, 9391-9401; Johannessen et al., 2010, Nature 468, 968-972; Nazarian et al., 2010 Nature 468, 973-977; Sun et al., 2011 Cancer Cell 18, 683-695; Villanueva et al., 2010 Cancer Cell 18 683-695. These findings suggest that inhibition of multiple kinases might be required for successful therapy.

### 2.4. Pancreatic Cancer Treatments

Finding effective therapies for pancreatic cancer continues to be a nearly insurmountable problem. Despite significant success with targeted therapies in other cancers, progress for pancreatic cancer has been disappointingly slow. Despite FDA approval in 2005 of the small molecule epidermal growth factor receptor (EGFR) inhibitor erlotinib, gemcitabine as a single agent or combinations of traditional chemotherapeutic agents remain the standard of care as erlotinib has not been widely embraced. Moore et al. 2005 J Clin Oncol 23 1. Other disappointing failures have occurred recently including therapies aimed at insulin-like growth factor 1 receptor IGF1R (ganitumab) or at smoothened (saridegenib), both of which were felt to have great preclinical promise. Yeh et al. 2007 Expert Opin Ther Targets 11 673-694. It is clear that better means of vetting agents preclinically for clinical testing are needed.

Protein kinases, with their key roles in promoting cell growth, proliferation, migration and survival, remain the most tractable targets for cancer therapy. Approximately 90% of pancreatic cancers have oncogenic Ras mutations but relatively few activating kinase mutations. Whole exome sequencing studies of primary and metastatic pancreatic cancer have found only 2 kinase genes with mutations (less than 3% of all mutations). Yachida et al., 2010 Nature 467 1114-1117; Jones et al. 2008 Science 321 1801-1806. Pancreatic cancer mutations are rare compared to mutations in 76 kinase genes in breast cancer and 141 kinase genes in lung cancer. Stephens et al. 2005 Nat Genet 37 590-592; Davies et al. 2005 Cancer Res 65 7591-7595. Although mutationally activated kinases have been the best examples of “Achille's heels” of several cancers, there is growing evidence that kinases may be activated by gene amplification (e.g., Her2) or chromosomal translocation (e.g., Bcr-Abl). Kinases activated by overexpression, gene fusions or truncations can still be susceptible to kinase inhibitor therapy. McDermott et al. 2009 J Clin Oncol 27 5650-5659. One of the best examples of this is the success of trastuzumab and other agents for the treatment of HER2-amplified breast cancer. In addition, a subset of non-small cell lung cancers show response to small molecule EGFR inhibitors without the presence of EGFR mutations. Furthermore, a recent study of 48,178 drug-cell line combinations (studied in an effort to identify genomic markers of drug sensitivity) demonstrated that a substantial number of potential drug-cell line combinations have no obvious mutational or genomic associations. Garnett et al. 2012 Nature 483 570-575. For example, genomic alterations could not be found to explain why RCC cell lines (another cancer where kinase mutations are uncommon), were sensitive to multiple SRC kinase inhibitors.

With over 130 kinase-specific inhibitors currently in Phase 1-3 clinical trials for different diseases, developing combination therapies for cancer subtypes should be a highly tractable goal. Currently, there is no feasible mechanism to effectively define the dynamic activity of the kinome in response to inhibitors. Such techniques could be used to assess global kinome behavior and its response to one or more small molecule inhibitors, leading to new and effective therapies to treat disease.

### 3. SUMMARY OF THE INVENTION

In particular non-limiting embodiments, the present invention provides a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity.

The invention also provides a method for detecting low abundant kinases in a sample comprising: loading a sample on a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity; washing the multi-analyte column to remove any unbound proteins; eluting any kinases bound to the multi-analyte column with a denaturing agent; and detecting the eluted kinases.

In particular non-limiting embodiments, the invention provides a method of selecting a kinase activity modulator, the method comprising the steps of: contacting a cell, a tissue, or an organism with a compound; contacting a protein extract from the cell, the tissue, or the organism with a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity; eluting any kinases bound to the solid supports with a denaturing agent; measuring levels of a plurality of the kinases detected; comparing the levels measured in step (d) to a standard level(s) to obtain a kinase profile; and using the kinase profile to select the kinase activity modulator.

In particular non-limiting embodiments, the invention provides a kit comprising: multi-analyte column with a first and a second layer wherein: the first layer comprises a first solid support having at least two different affinity ligands with specific kinase binding affinity; and the second layer comprises a second solid support having at least two different affinity ligands with non-specific kinase binding affinity; and instructions for use in measuring level of a plurality of kinases in a subject who has cancer or been previously treated with a chemotherapy regimen.

In particular non-limiting embodiments, the invention provides compounds having the structures shown in Section 5.4 below.

### 4. BRIEF DESCRIPTION OF THE FIGURES

FIG. 1A-1I. Kinome profiling of TNBC reveals elevated ERK signaling. (FIG. 1A) Experimental strategy for the rational design of kinase inhibitor combination therapies. To define kinome inhibitor response signatures, expression profiling is integrated with kinase affinity capture and MS quantitative assessment of the activation state of the kinome. RNAi is used to analyze kinase function in survival response to inhibitors. (FIG. 1B) Venn diagram showing kinase expression defined by RNA-seq across patient TNBC and MDA-MB-231 and SUM159 cell lines. (FIG. 1C) Venn diagram showing kinases captured and identified by MIB-based proteomics across patient-sample TNBC and MDA-MB-231 and SUM159 cell lines. (FIG. 1D) The distribution and (FIG. 1E) the overlap of expressed and MIB-bound kinases across the TNBC patient sample and the MDA-MB-231 and SUM159 cell lines. The kinome trees EW based on the layout of Manning et al. and poster that appeared in Science Magazine. Manning et al. 2002 Science, 298 1912-1934. MIB/MS captures 50-60% of the expressed kinome as defined by RNA-seq. See Table 3 for MS identifications of protein kinases. (FIG. 1F) RAF/MEK/ERK pathway activated in patient TNBC tumors. Quantitative comparison of patient TNBC to matched uninvolved mammary tissue using MIB/MS. The line graphs show iTRAQ determined quantitative changes in MIB binding as a ratio of tumor/uninvolved. Ratio <1 denotes decreased MIB binding and >1 increased MIB binding of kinase in tumor versus control tissue. (FIG. 1G) Immunoblotting confirms an activated RAF/MEK/ERK pathway in TNBC cell lines and TNBC patient samples. (FIG. 1H) RTK array analysis of patient TNBC tumors reveals multiple Tyr phosphorylated RTKs, including EGFR and PDGFRβ. FIG. 1(I) shows number of kinases bound to each affinity bead in the multiplexed inhibitor beads using 5 mg extract of cellular protein. Specifically, 13 kinases bound the Bis-X bead; 15 kinases bound the lapatinib bead; 26 kinases bound the SB203580 bead; 46 kinases bound the dasatinib bead; 88 kinases bound the purvananol B bead; 104 kinases bound the PP58 bead; and 124 kinases bound the VI16832 bead. Between all the beads 186 unique kinases were bound and a total of 416 kinases were bound (some kinases bound to multiple beads). Loading additional material allows deeper recovery of the kinome (more unique kinases). See Section 5.6.1 Bead Design, Section 6.18 Chromatography, Section 6.22 Mass Spectrometry for the experimental details.

FIG. 2A-2J. Reprogramming of the kinome in response to MEK inhibition. (FIG. 2A) Growth inhibition of SUM159 cells in response to 5 μM AZD6244 or U0126. Triplicate experiments+SD. (FIG. 2B) Reactivation of MEK and ERK in the continued presence of 5 μM AZD6244 shown by western blot. (FIG. 2C) Loss of ERK regulated feedback of the RAF/MEK/ERK pathway and downstream signaling. SUM159 cells were treated with 5 μM AZD6244 for 12 h and kinome phosphorylation analyzed using MIB/MS. (FIG. 2D) Activation and repression of the kinome in response to MEK inhibition in SUM159 cells. Line graphs show iTRAQ determined quantitative changes in MIB binding as a ratio of AZD6244/DMSO. Ratio <1 denotes decreased MIB binding and >1 increased MIB binding of kinases in treated versus control cells. (FIG. 2E) MEK2 and ERK1 escape AZD6244 inhibition. MIB/MS profile of RAF-MEK-ERK binding from SUM159 cells treated with 5 μM AZD6244 for 4, 12 and 24 h or 5 μM U0126 for 24 h. (FIG. 2F) MEK2 and ERK1 promote survival following MEK inhibition. siRNA knockdown of MAPK signaling components in SUM159 cells shows loss of MEK2, but not MEK1, inhibits growth in the presence of U0126. (FIG. 2G) Kinome response signature for MEK inhibition in SUM159 cells. Triplicate MIB/MS runs of SILAC labeled SUM159 cells ±5 μM AZD6244 or U0126 relative to DMSO. Error bars represent mean+SD where kinases are significant at FDR of 0.05. (FIG. 2H) Kinome map of AZD6244 response (light gray: downregulated, dark gray: upregulated) as determined by MIB/MS and RTK arrays. (FIG. 2I) Increased tyrosine phosphorylation of RTKs in response to MEK inhibition. SUM159 cells treated with 5 μM AZD6244 for 24 h and analyzed by RTK array. (FIG. 2J) Concentration-dependent RTK reprogramming in response to MEK inhibition. Induction of RTK expression and activity in SUM159 cells following treatment with increasing dose of AZD6244 for 24 h was determined by western blot.

FIG. 3A-3H. AZD6244-induced kinome reprogramming is target specific and involves rapid, stable transcriptional upregulation of RTKs and cytokines. (FIG. 3A) AZD6244 treatment over time reveals an early response in which ERK is inhibited and MKP3 accumulates. With prolonged AZD6244 treatment, increased RTK expression and downstream survival signaling occurs, coinciding with the reactivation of RAF/MEK/ERK signaling. (FIG. 3B) Time-dependent increase in RTK and (FIG. 3C) cytokine gene expression in SUM159 cells following 5 μM AZD6244 treatment determined by qRT-PCR over a 48 h time course. (FIG. 3D) Prolonged treatment of SUM159 cells with 5 μM AZD6244 leads to stable upregulation of RTKs. SUM159 cells were treated with DMSO or 5 μM AZD6244 for 4, 24, 48 or 72 h and RTK tyrosine phosphorylation compared to SUM159-R cells using RTK antibody arrays. (FIG. 3E) Treatment with 5 μM AZD6244 enhances phosphorylation of PDGFRβ at multiple sites, including the activation loop as shown by western blot. (FIG. 3F) Generation of AZD6244-resistant SUM159 cells following stable treatment with 5 μM AZD6244. Increased cell growth of SUM159-R cells compared to SUM159 cells treated with 5 μM AZD6244 for 72 h determined by cell counts (*p-value <0.001). (FIG. 3G) Maintenance of RTK reprogramming in SUM159-R cells accompanied by increased survival signaling. Kinome reprogramming in SUM159 cells treated with DMSO or 5 μM AZD6244 for 4 h was compared to SUM159-R cells by western blot. (FIG. 3H) AZD6244 and BEZ235 are target-specific in their reprogramming of kinome response and growth arrest. Treatment of SUM159 cells with 50 nM BEZ235 induces a unique kinase response different from AZD6244, even though both inhibit cell growth. Error bars represent triplicate experiments ±S.D.

FIG. 4A-4Q. AZD6244-mediated loss of ERK1/2 causes rapid degradation of c-Myc, destabilization of Myc-Max complexes and promotion of RTK expression. (FIG. 4A) Loss of ERK1/2 activity following AZD6244 treatment promotes c-Myc degradation. SUM159 cells were treated with 5 μM AZD6244 for 72 h and ERK-mediated phosphorylation of c-Myc at Ser62 and c-Myc degradation monitored by western blot. (FIG. 4B) Stable suppression of c-Myc RNA levels following AZD6244 treatment. MDA-MB-231 and SUM159 cells were treated with 5 μM AZD6244 for 48 h and c-Myc gene expression determined using qRT-PCR.

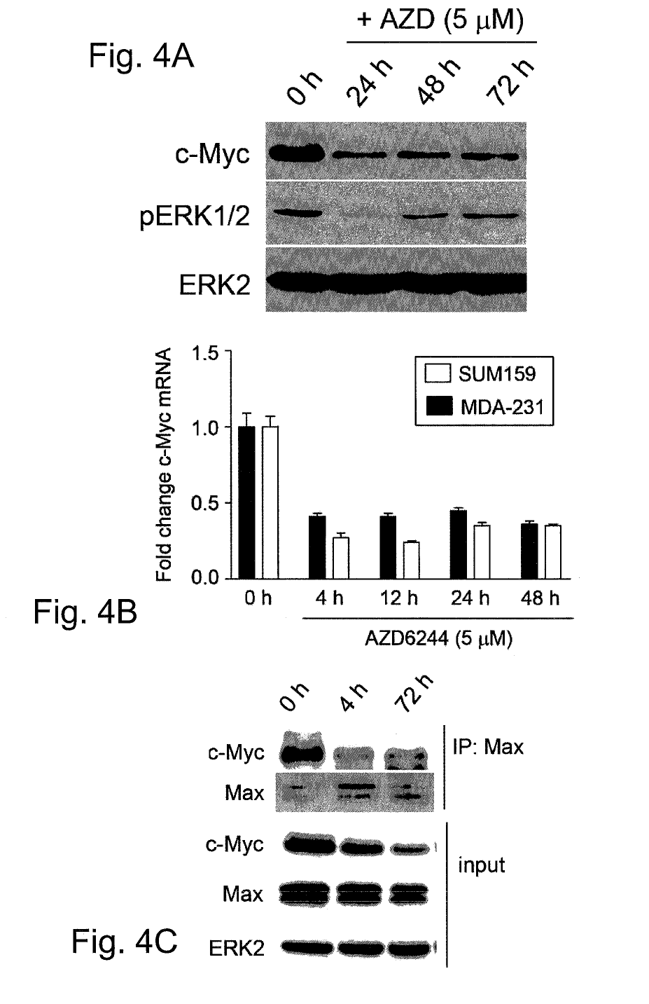

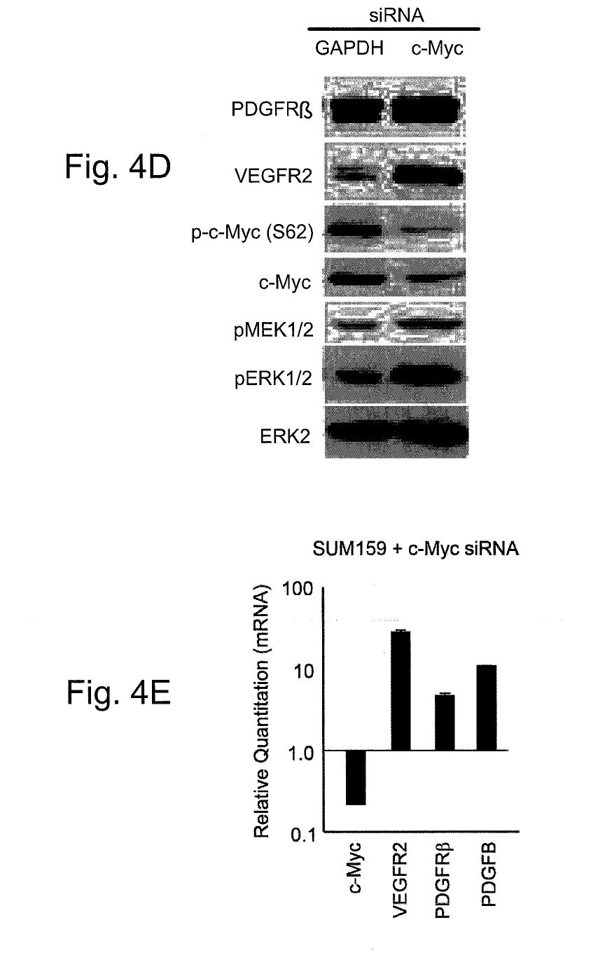

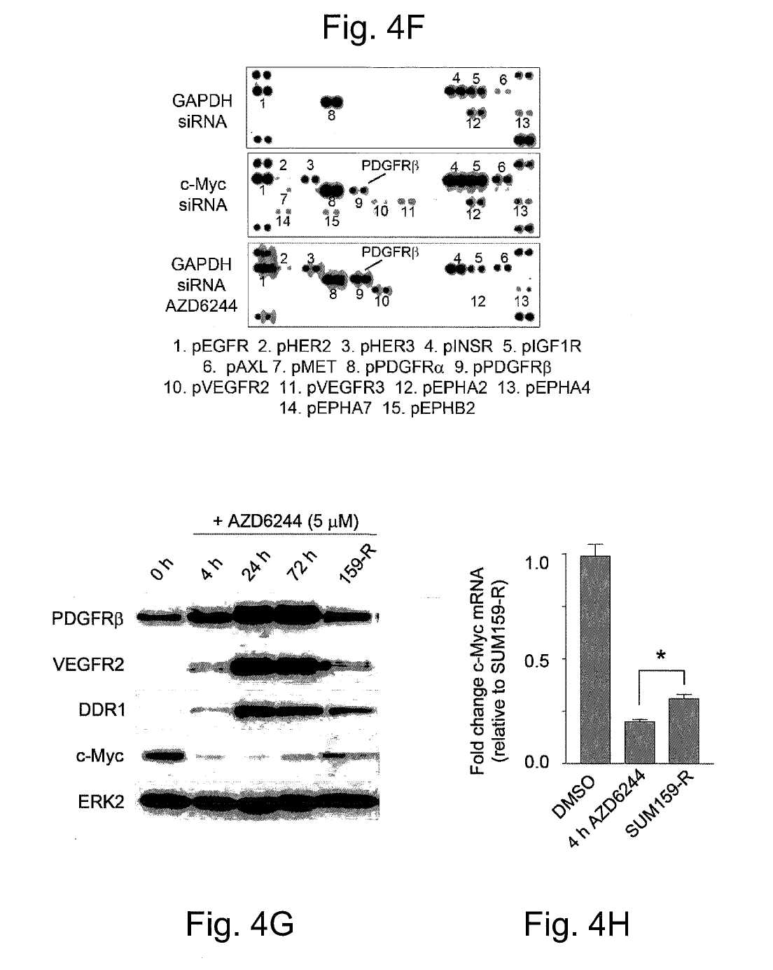

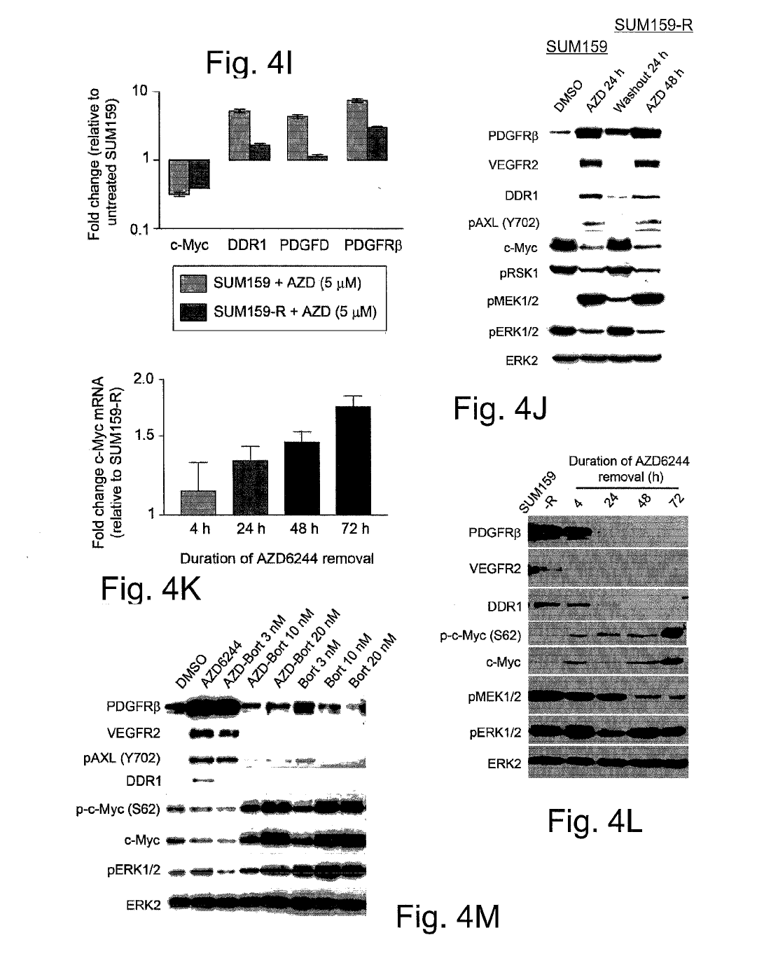

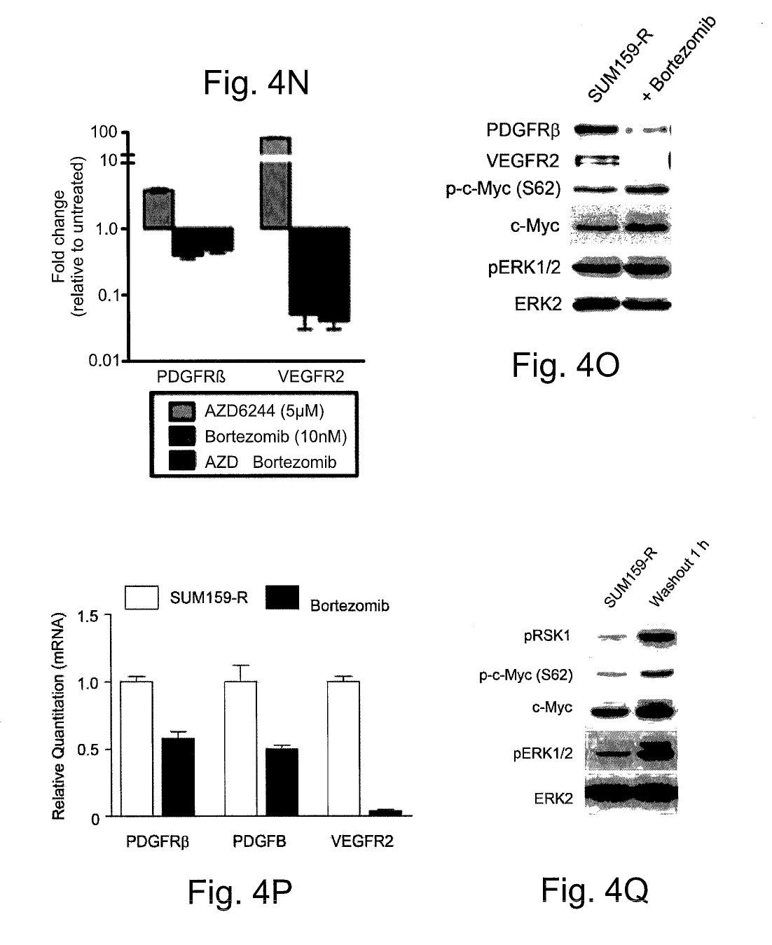

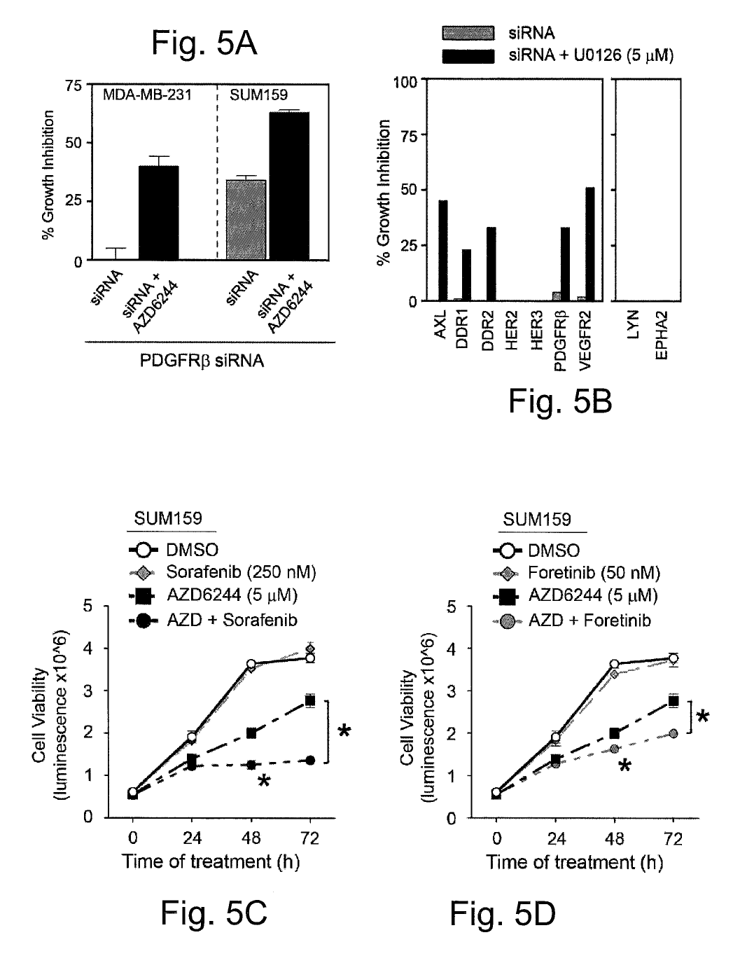

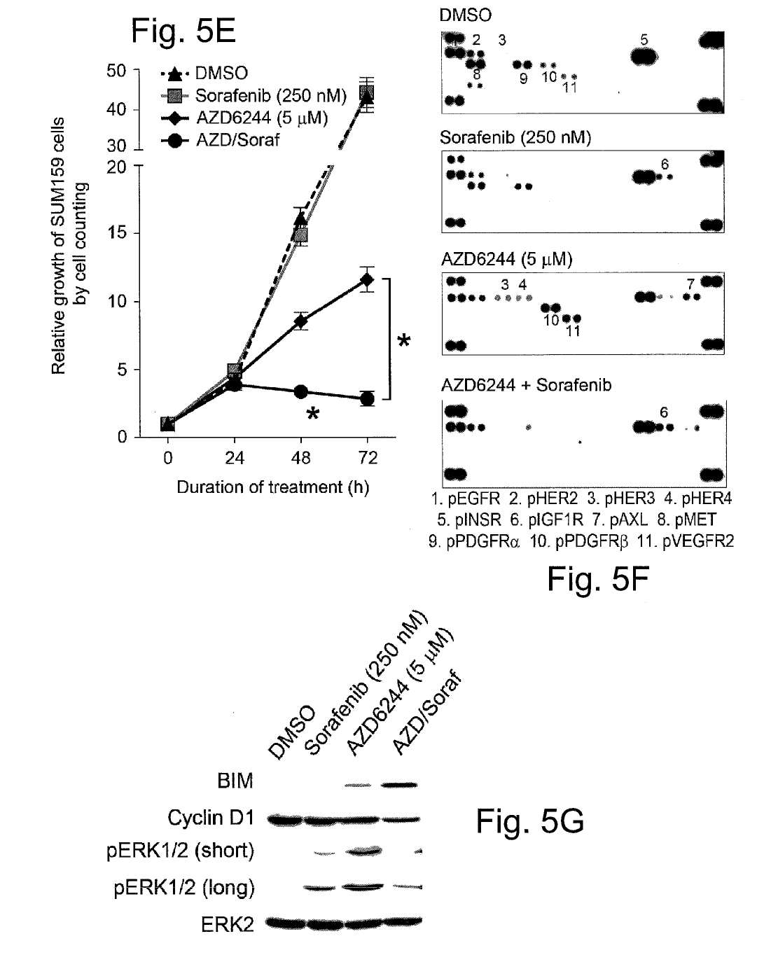

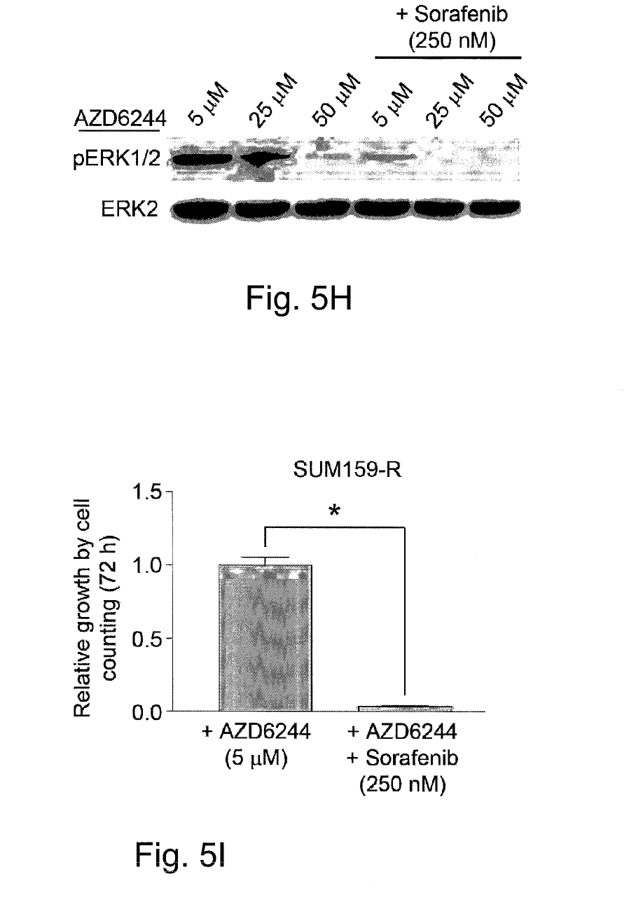

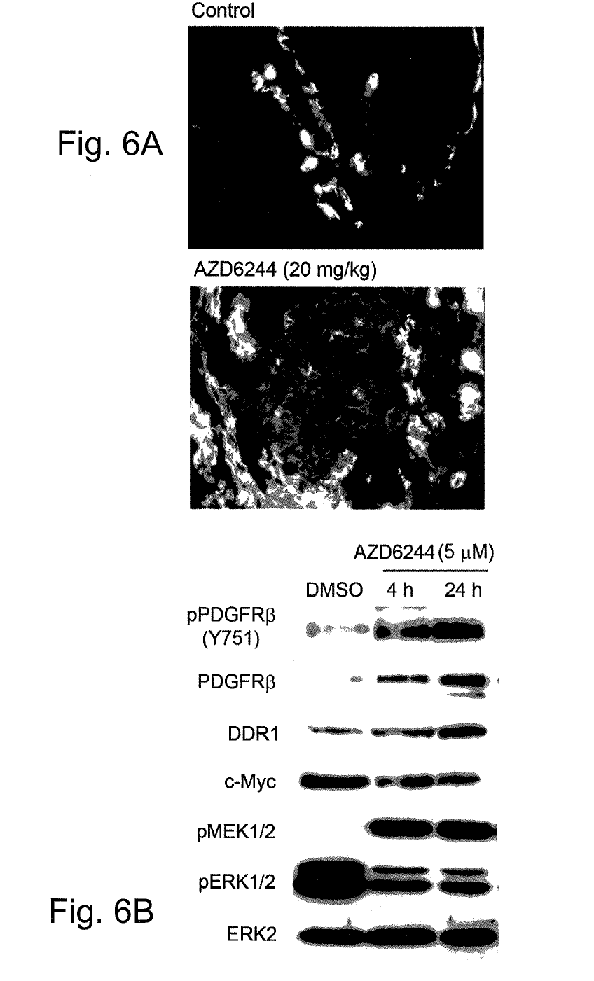

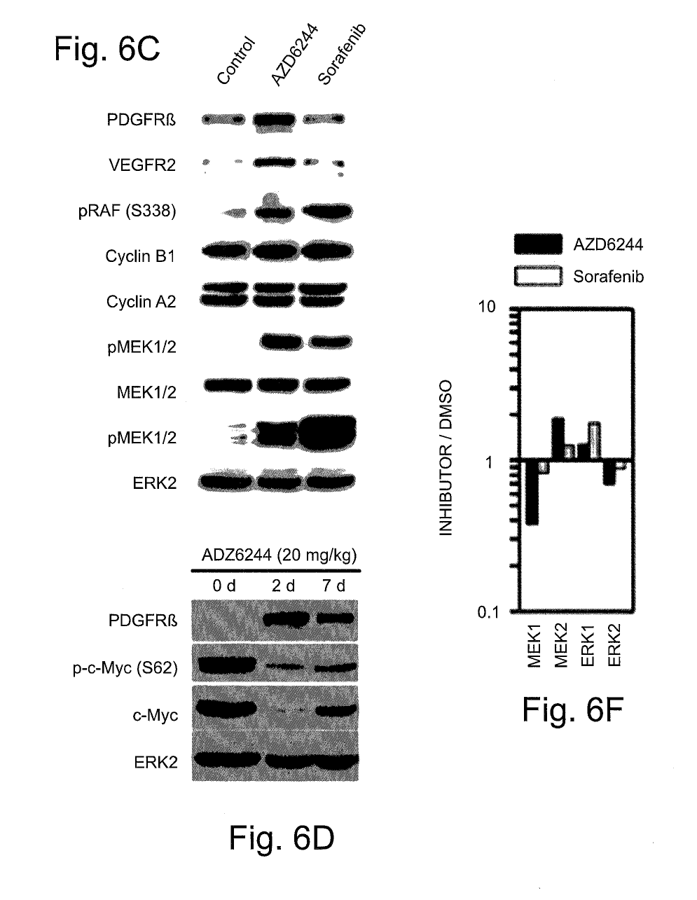

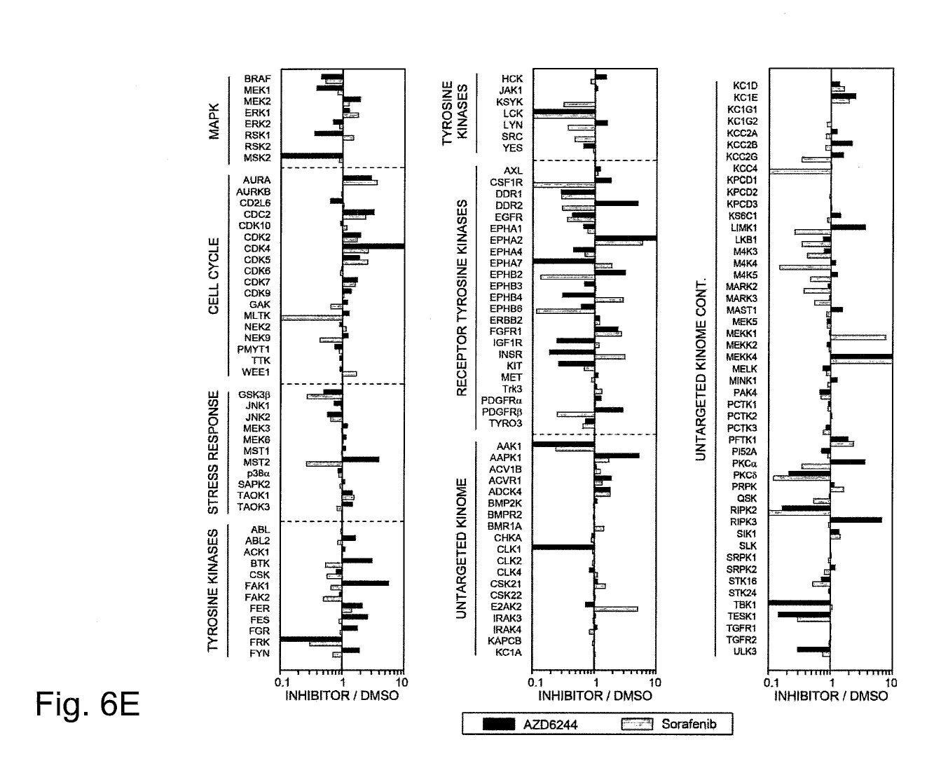

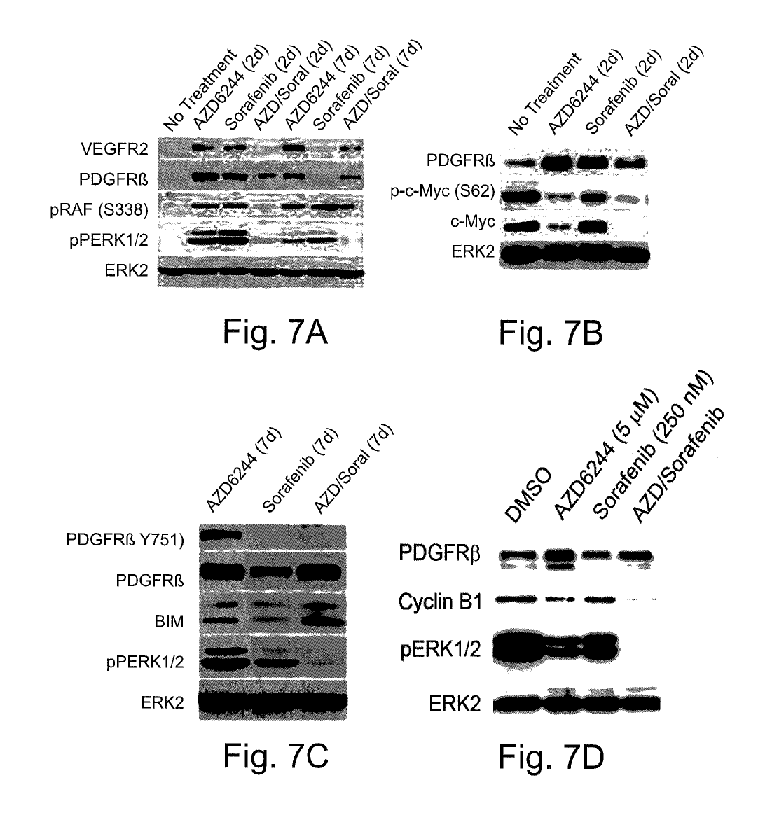

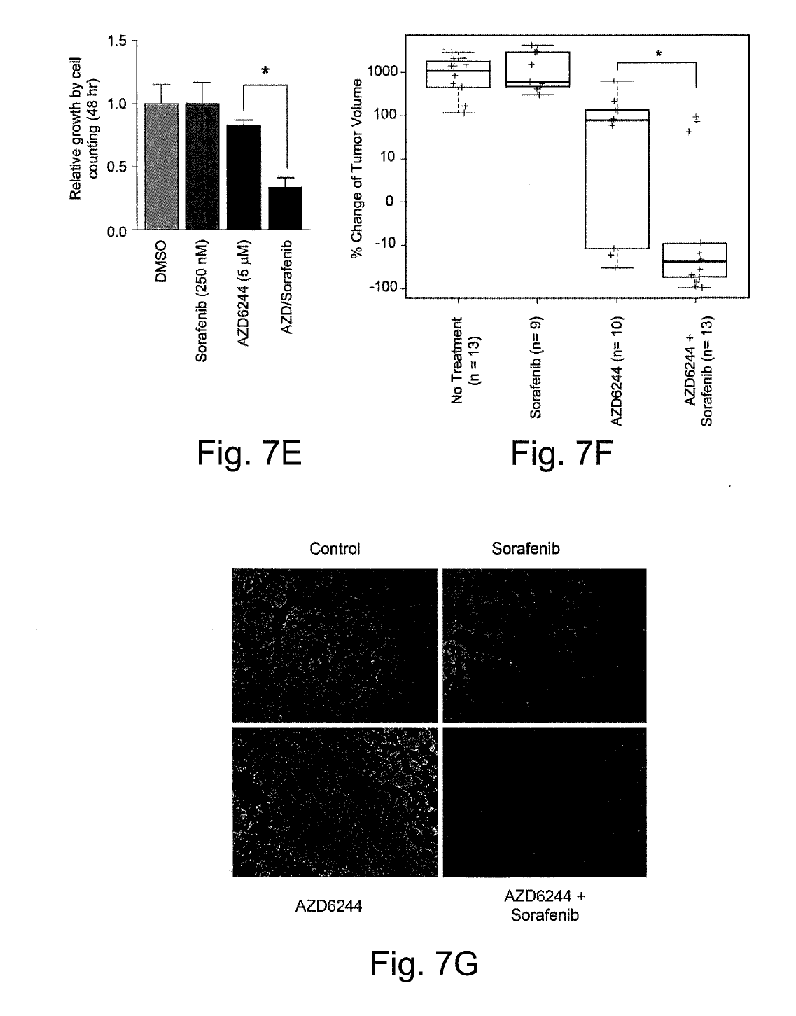

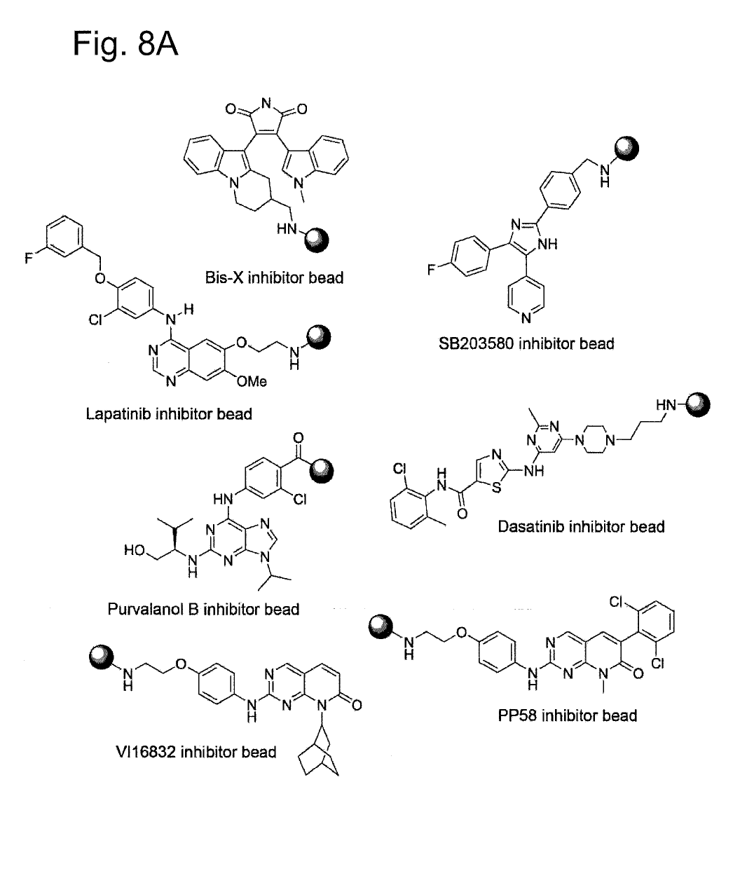

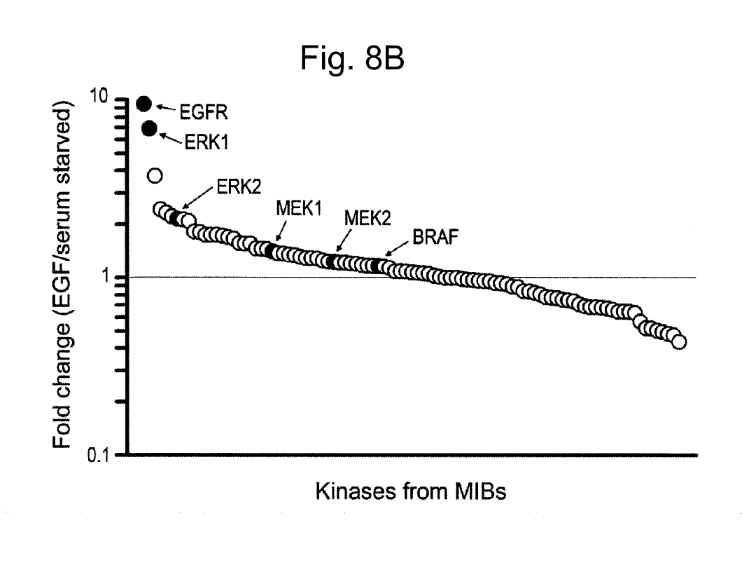

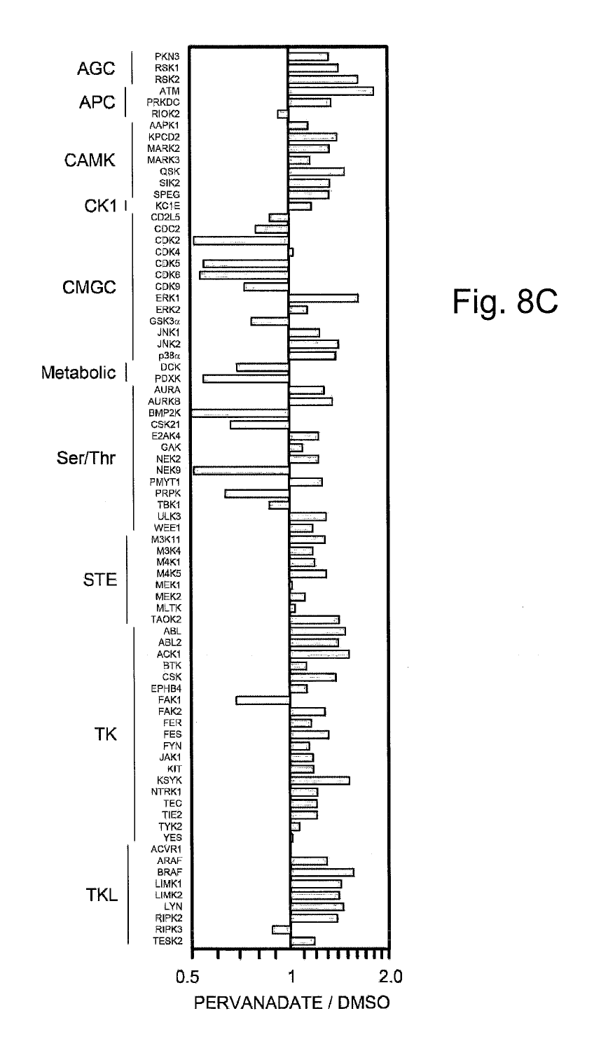

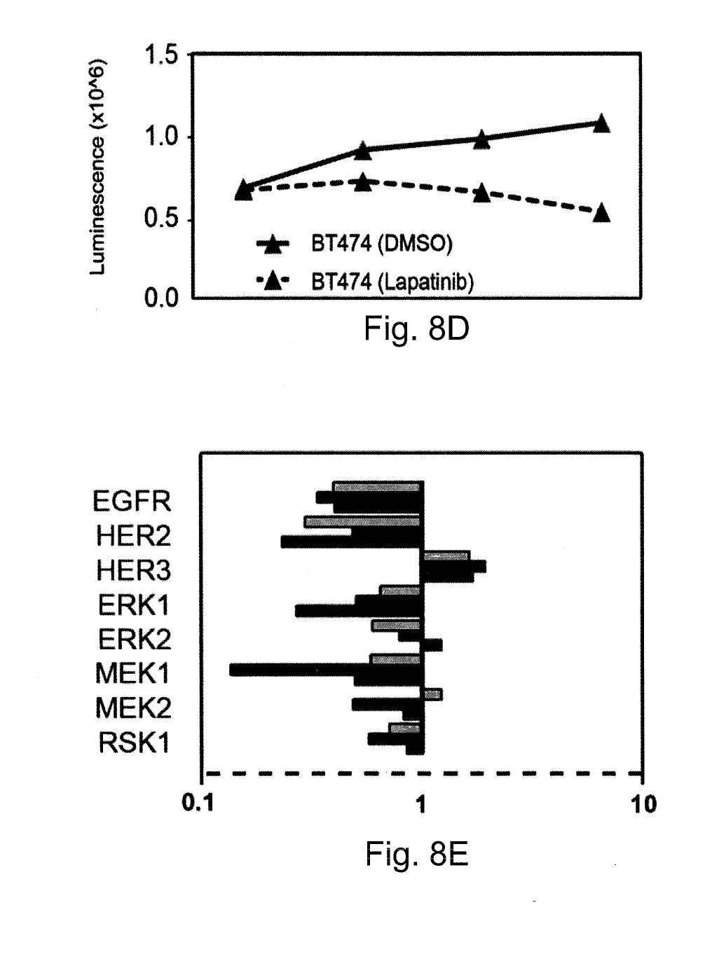

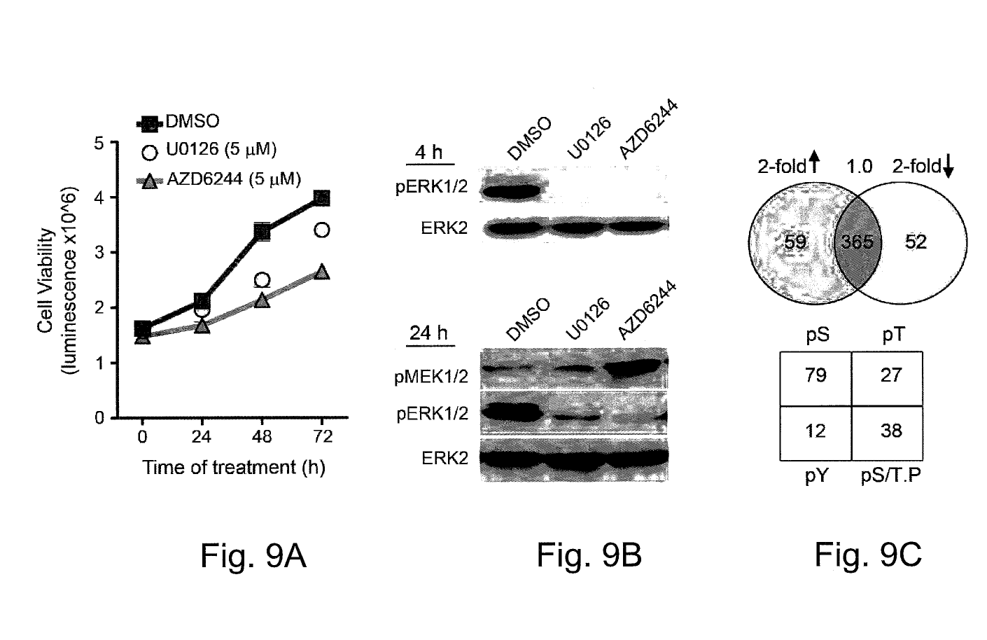

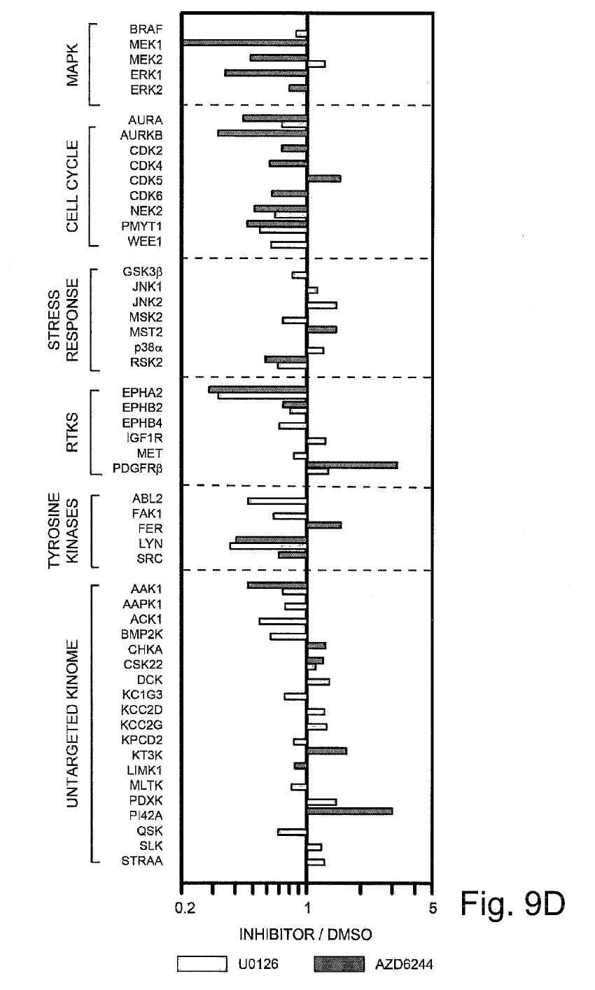

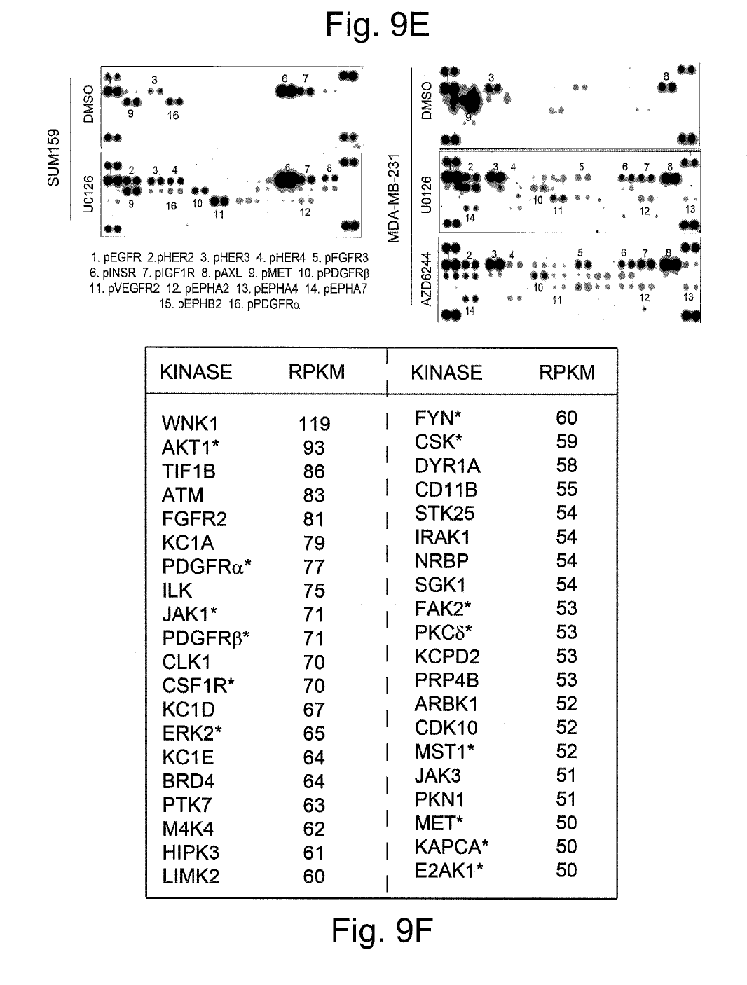

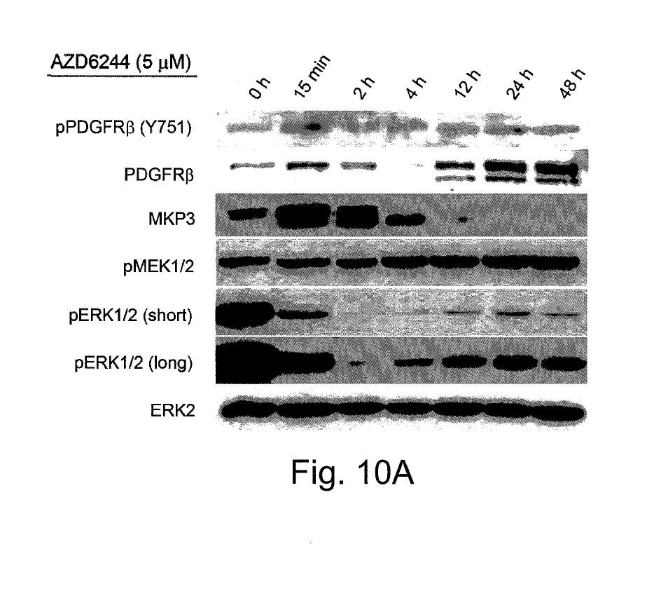

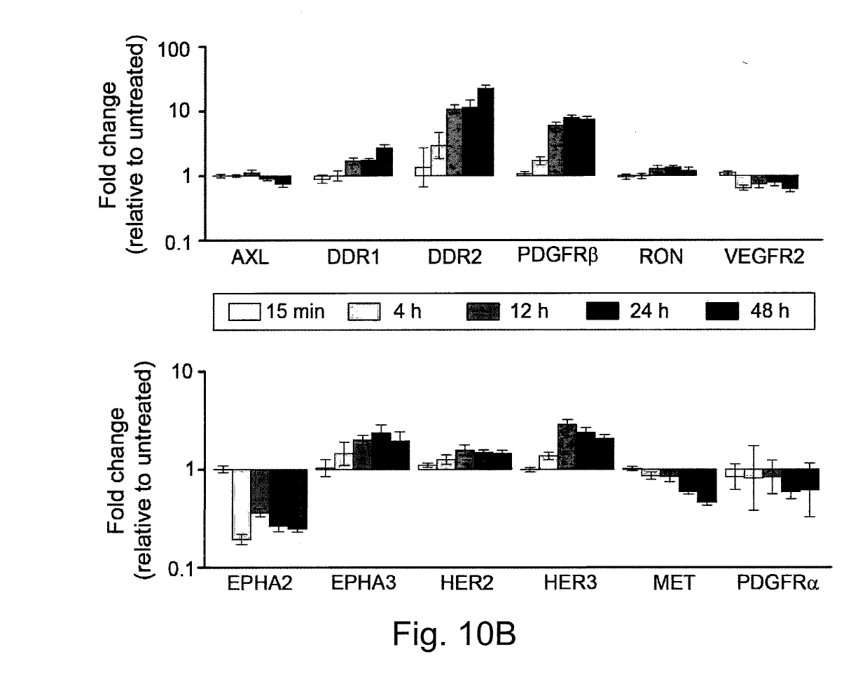

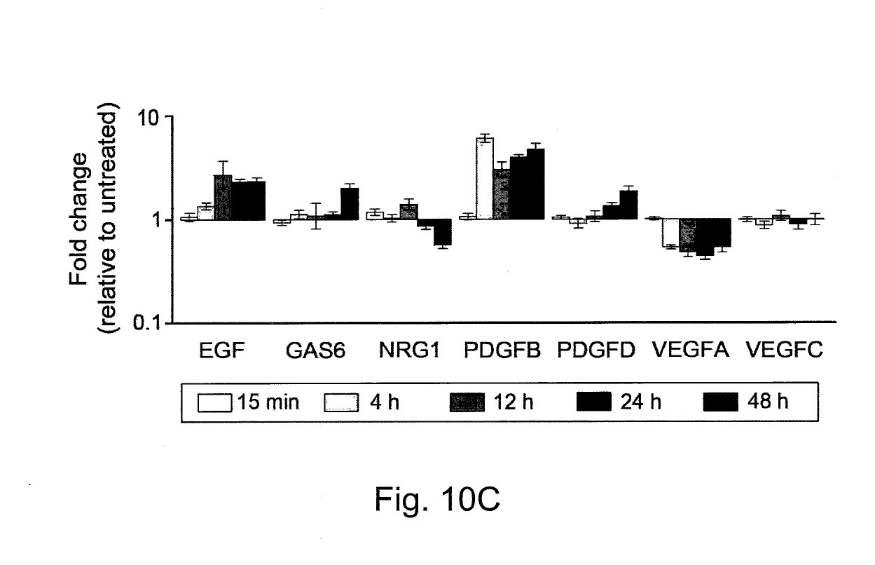

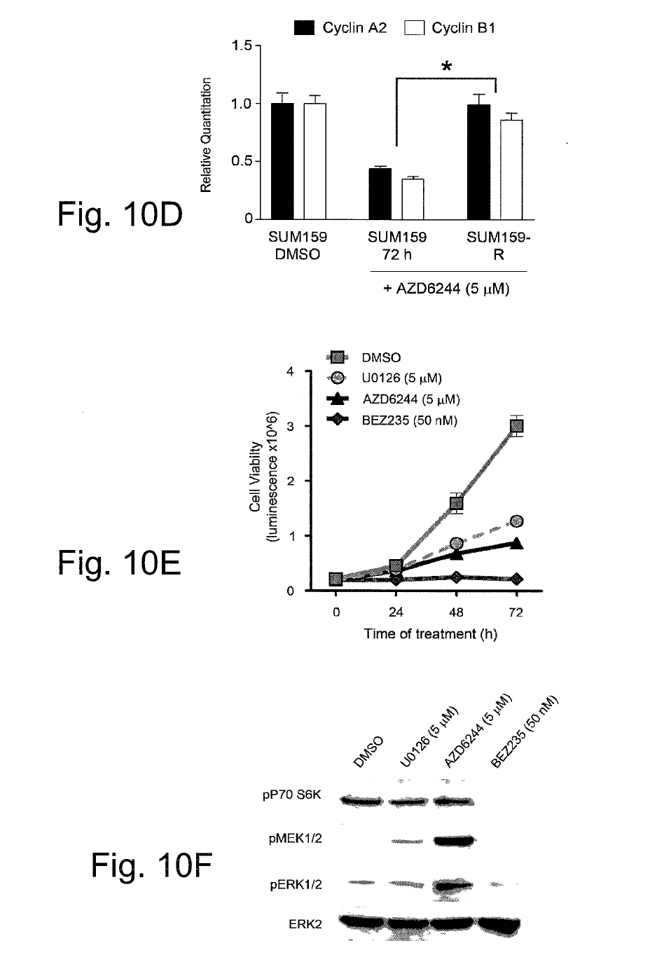

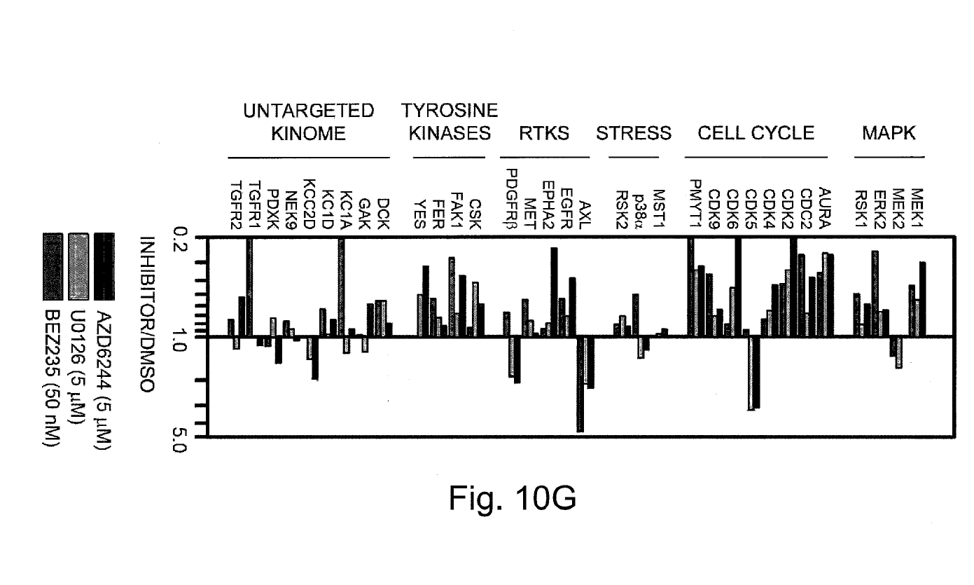

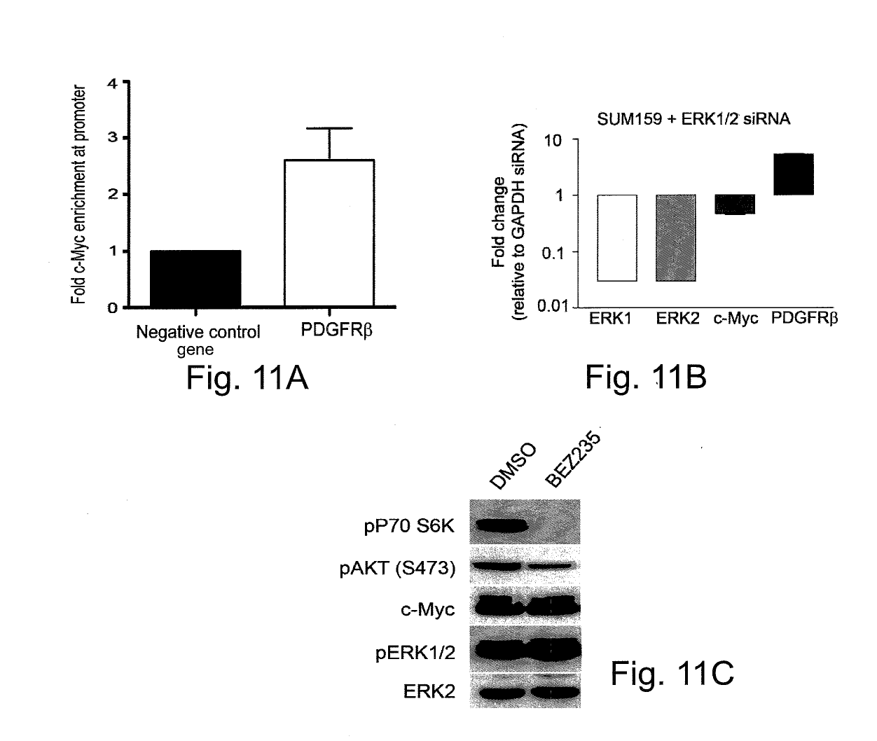

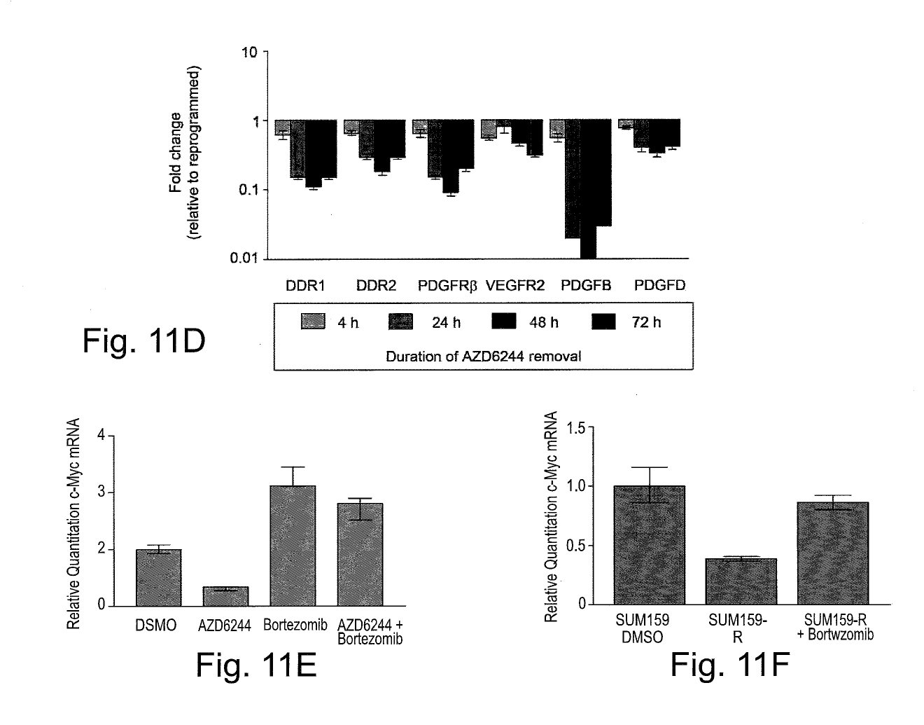

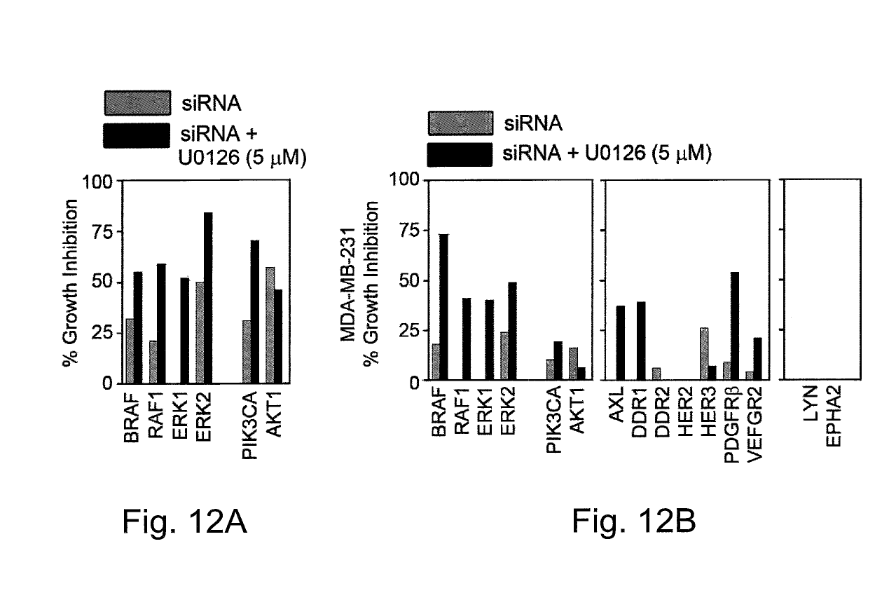

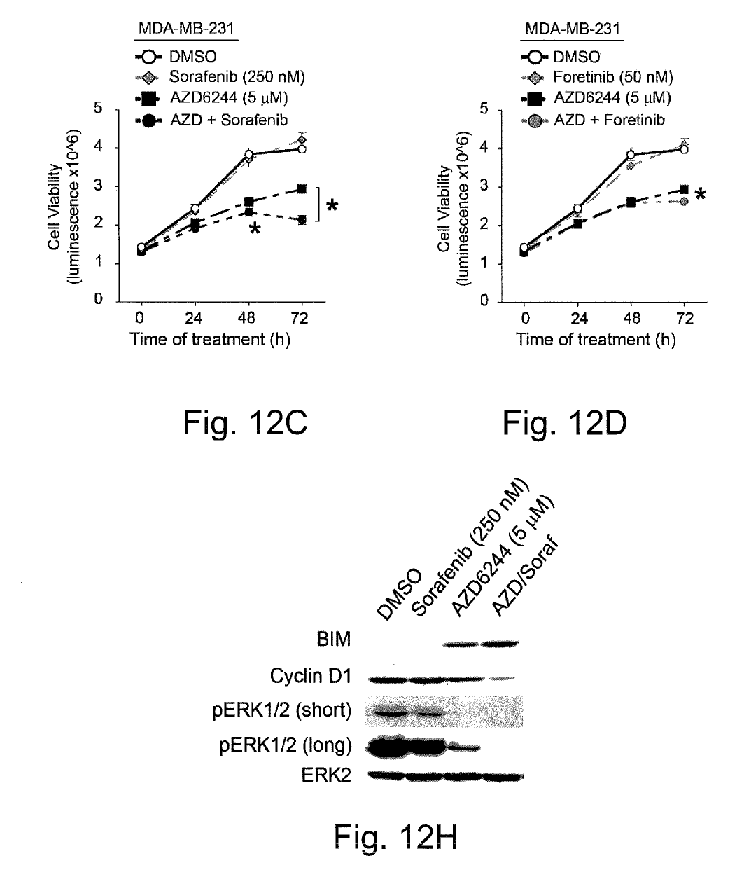

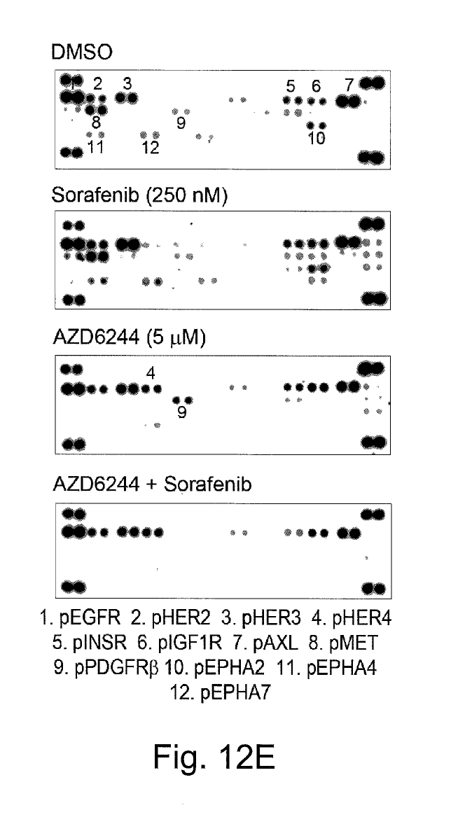

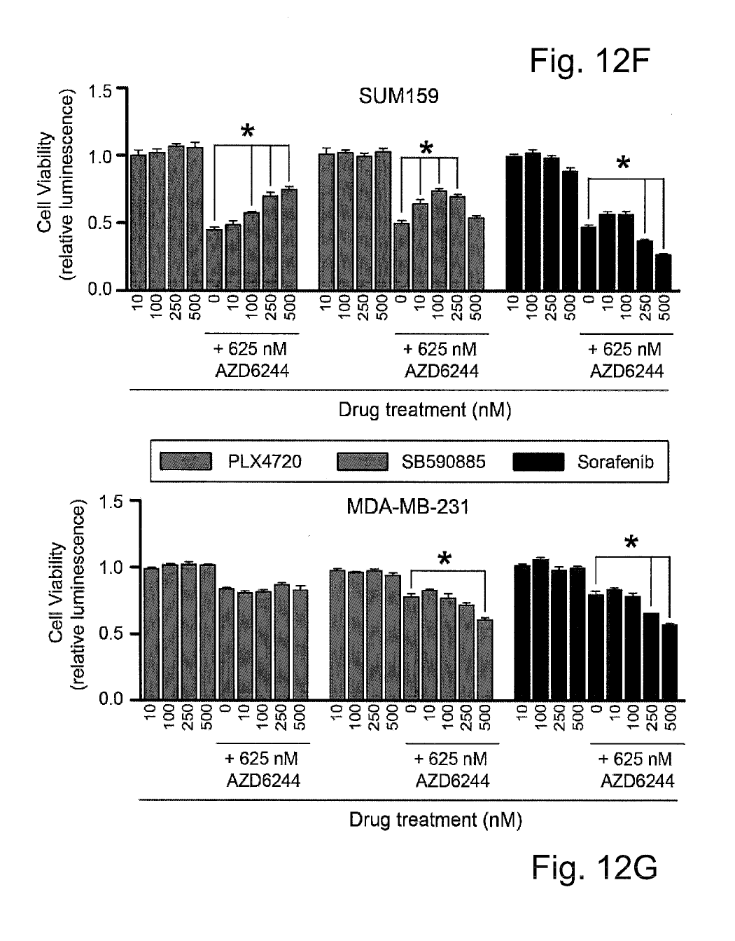

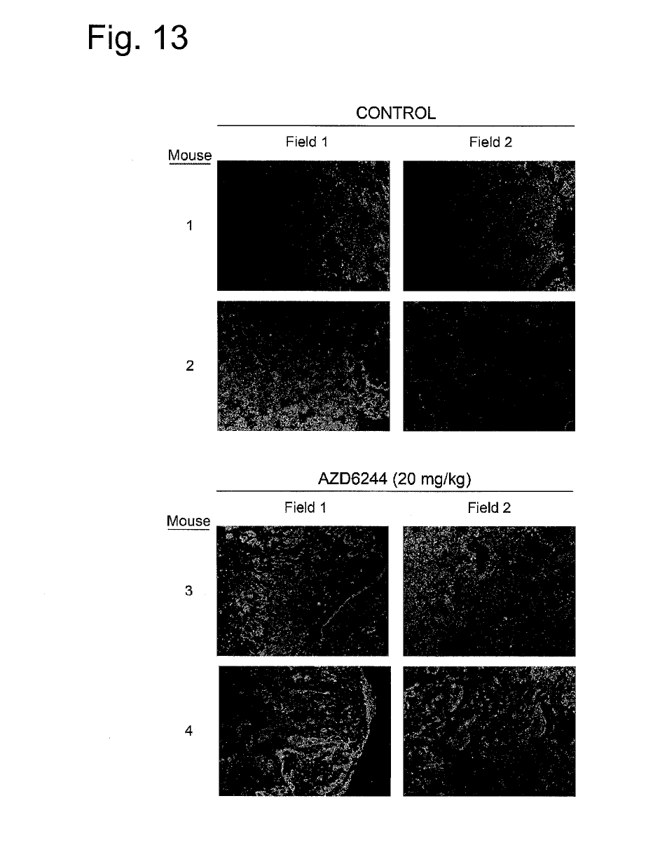

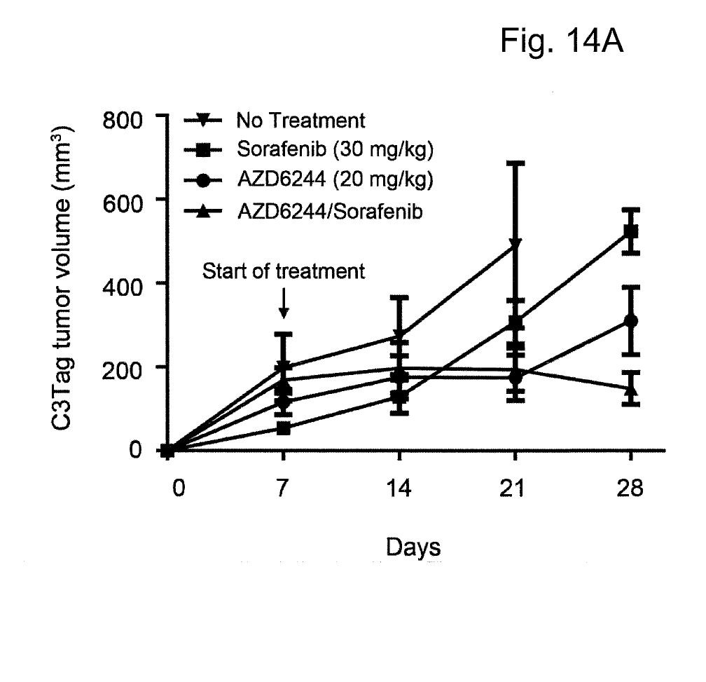

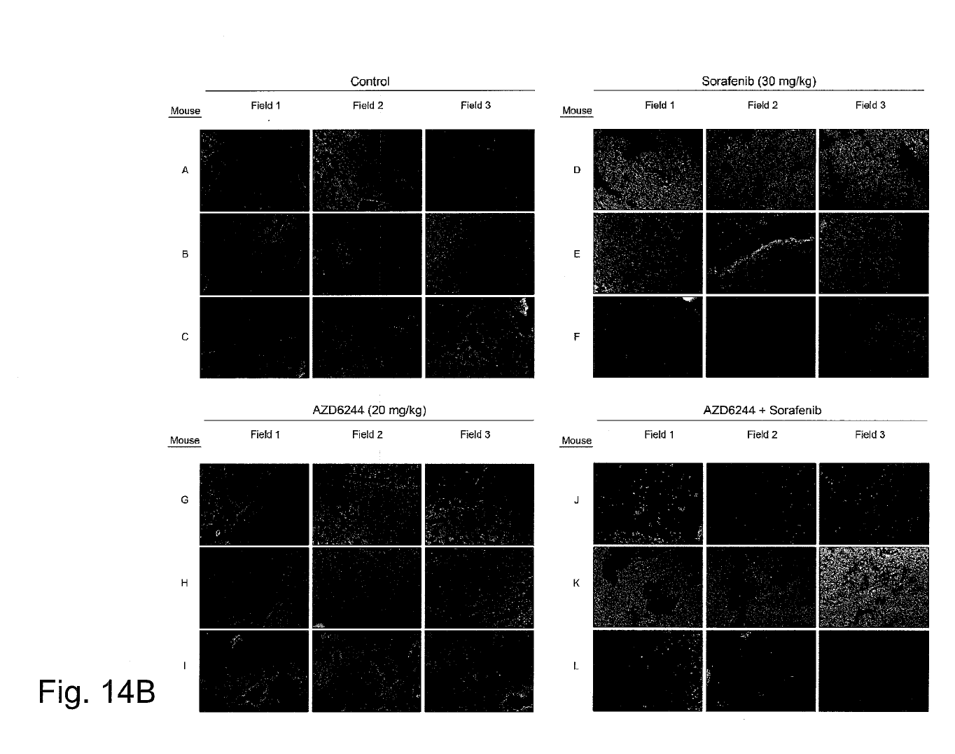

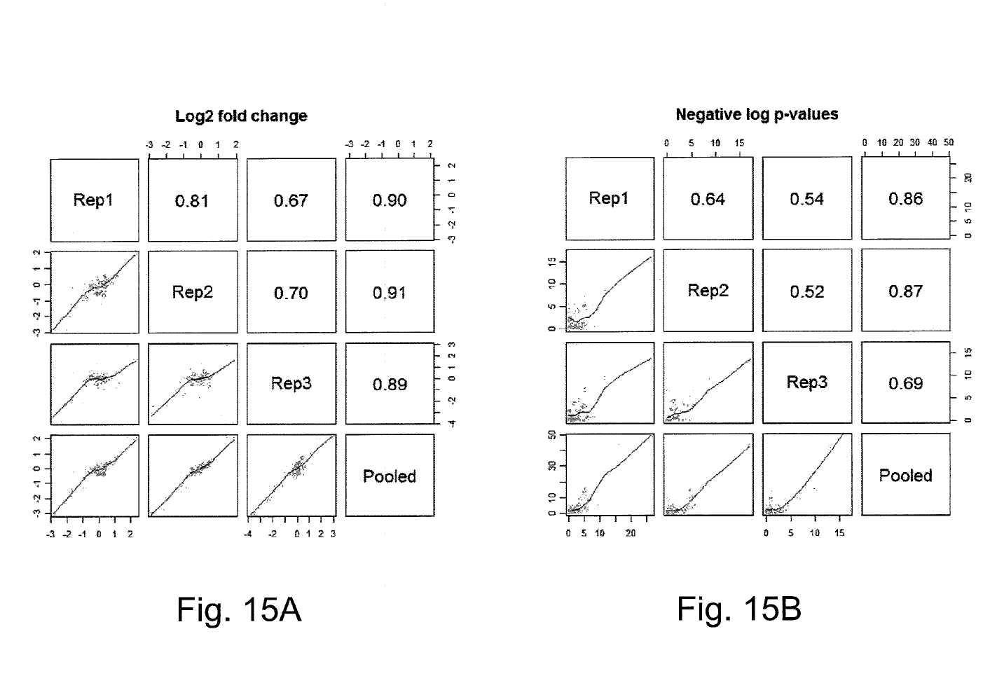

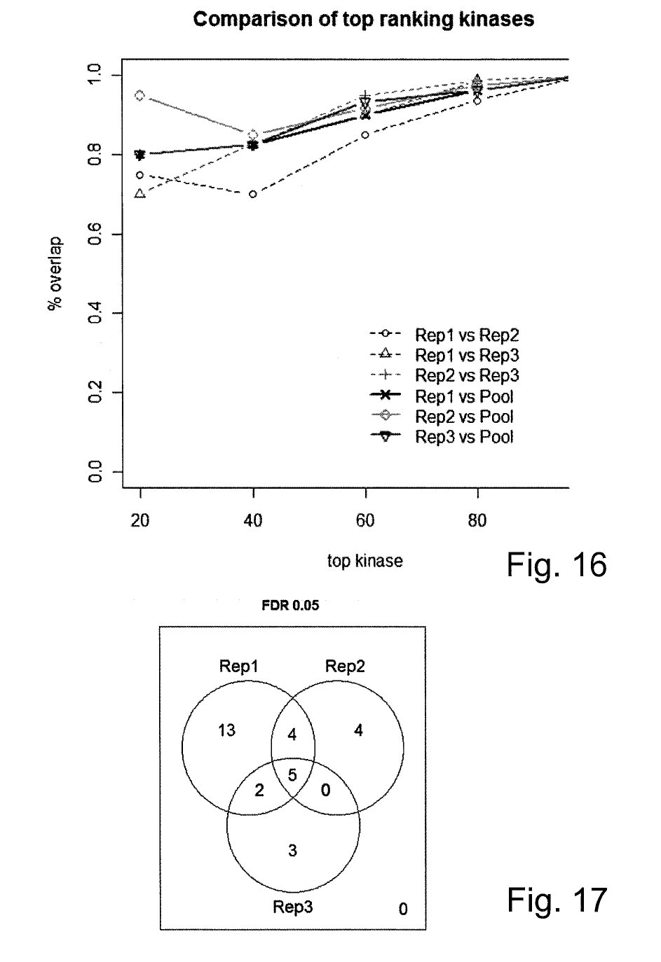

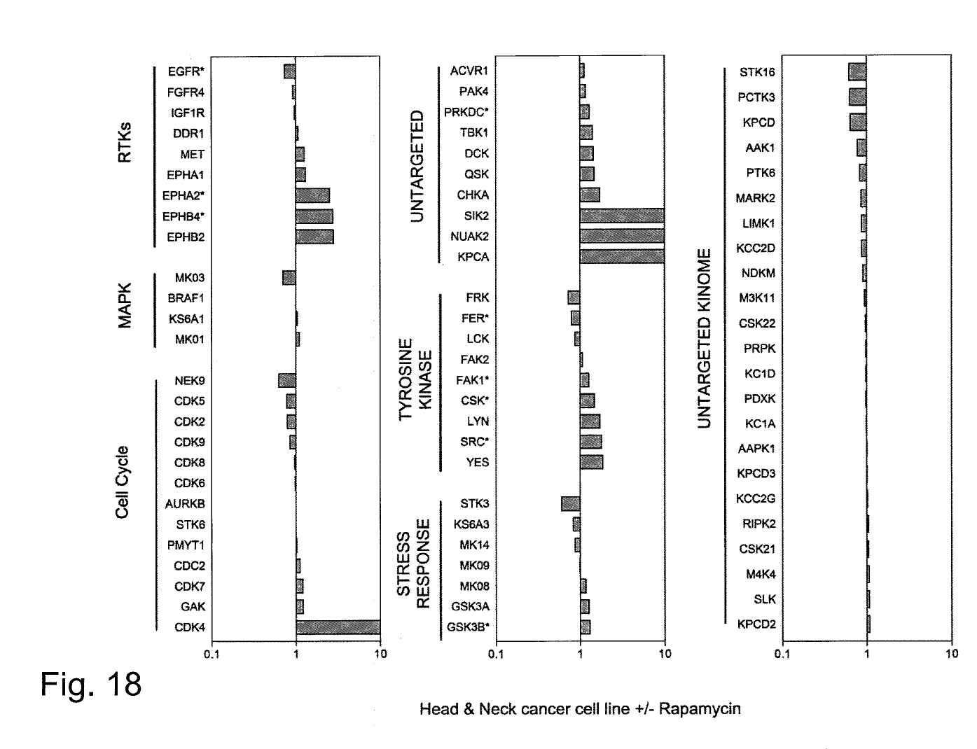

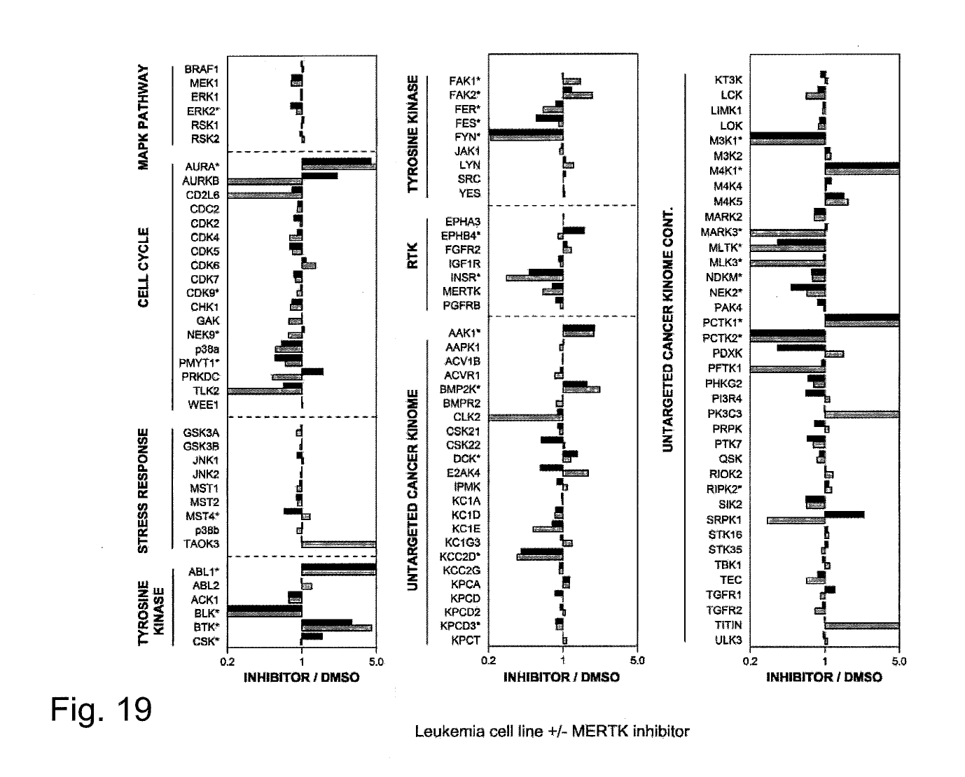

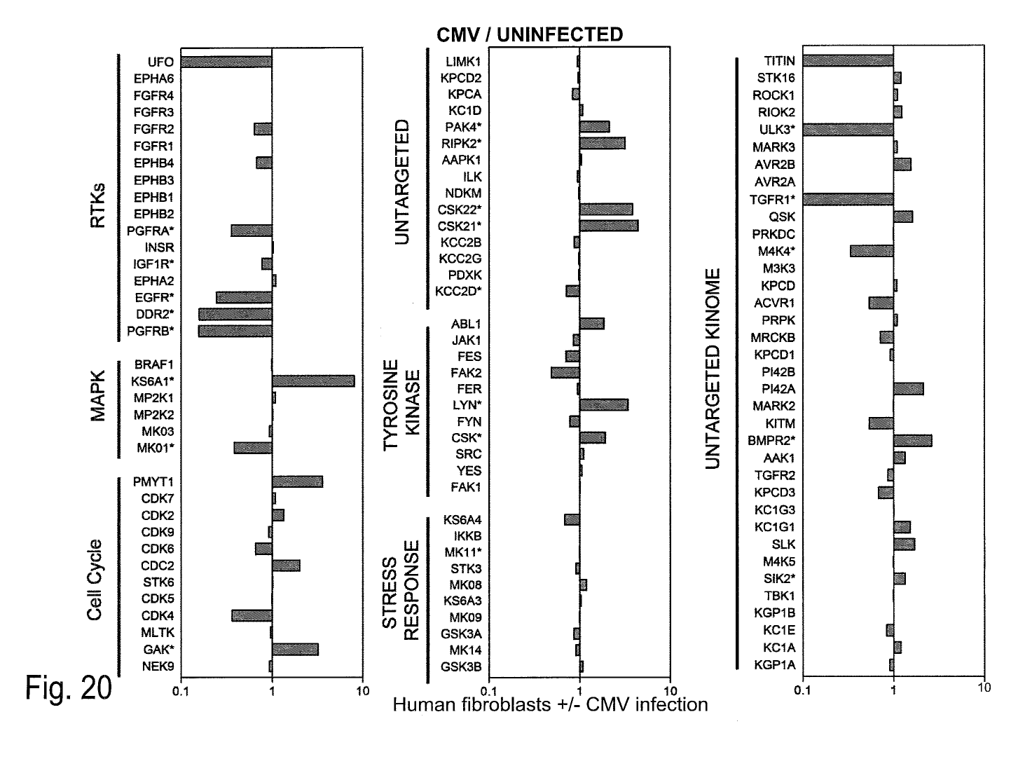

(FIG. 4C) Disruption of Myc-Max complexes following AZD6244 treatment. SUM159 cells were treated with 5 μM AZD6244 for 0, 4 and 72 h. Nuclear extracts were immunoprecipitated with anti-Max antibodies and immunoblotted using anti-Myc and anti-Max antibodies. (FIG. 4D) RNAi knockdown of c-Myc upregulates PDGFRβ and VEGFR2 expression and promotes ERK1/2 signaling. SUM159 cells were transfected with c-Myc or GAPDH siRNA for 72 h and kinome reprogramming determined by western blot. (FIG. 4E) RNAi knockdown of c-Myc for 72 h in SUM159 cells upregulates gene expression of PDGFRβ, VEGFR2 and PDGFB as determined by qRT-PCR. (FIG. 4F) RNAi knockdown of c-Myc for 72 h in SUM159 cells induces an RTK tyrosine phosphorylation similar to AZD6244 treatment with an increase in PDGFRβ and VEGFR2 phosphorylation. (FIG. 4G) c-Myc protein levels partially return in SUM159-R cells, while AZD6244-mediated RTK reprogramming is reduced but still maintained. SUM159 cells were treated with 5 μM AZD6244 for 0, 4, 24 or 72 h and RTK and c-Myc levels compared to SUM159-R cells by western blot. (FIG. 4H) Increased c-Myc RNA levels in SUM159-R cells relative to AZD6244 treated SUM159 cells. SUM159 cells were treated with DMSO or 5 μM AZD6244 for 4 h and c-Myc gene expression compared to SUM159-R cells using qRT-PCR (*p-value <0.001). (FIG. 4I) AZD6244-induced RTK expression levels maintained at reduced levels in SUM159-R cells. Gene expression profiles of RTK reprogramming comparing SUM159 cells treated with 5 μM AZD6244 for 24 h or SUM159-R cells relative to DMSO-treated cells. Gene expression was determined by qRT-PCR. (FIG. 4J) c-Myc stabilized by RTK-mediated ERK activation in SUM159-R cells. RTK reprogramming and c-Myc levels were determined by western blot comparing SUM159 cells treated with DMSO or 5 μM AZD6244 for 24 h to SUM159-R cells. (FIG. 4K) Washout of AZD6244 restores c-Myc RNA levels. AZD6244 was removed from the media of SUM159-R cells and c-Myc RNA levels determined by qRT-PCR over 72 h. (FIG. 4L) Removal of AZD6244 results in stabilization of c-Myc protein and reversal of RTK reprogramming. AZD6244 was removed from the media of SUM159-R cells over a 72 h period and RTK reprogramming determined by western blot. (FIG. 4M and FIG. 4N) Stabilization of c-Myc protein levels using proteasome inhibitor bortezomib prevents AZD6244-mediated kinome reprogramming. SUM159 cells were treated with AZD6244 (5 μM) or bortezomib (3, 10 or 20 nM) alone or in combination for 24 h. Bortezomib blocked the AZD6244-dependent induction of RTKs as shown by western blot and qRT-PCR. (FIG. 4O and FIG. 4P) Bortezomib treatment of SUM159-R cells stabilizes c-Myc and reverses RTK reprogramming. SUM159-R cells were treated with 10 nM bortezomib for 24 h and c-Myc protein levels and reversal of RTK induction shown be western blot. Gene expression levels of c-Myc and RTKs was determined by qRT-PCR. (FIG. 4Q) RTK-mediated reactivation of ERK activity is not complete in the continued presence of AZD6244. AZD6244 (5 μM) was removed from media of SUM159-R cells for 1 h and ERK1/2 phosphorylation of c-Myc and RSK1 determined by western blot. Error bars represent triplicate experiments ±S.D.

FIG. 5A-5I RTK inhibition synergizes to enhance AZD6244-induced growth arrest (FIG. 5A) RNAi knockdown of PDGFRβ in AZD6244-treated cells enhances MEK inhibition-induced growth arrest in TNBC cell lines. PDGFRβ knockdown was performed in presence or absence of 1.25 μM AZD6244 in MDA-MB-231 and SUM159 for 96 h and cell growth monitored using Cell-Titer Glo. (FIG. 5B) RNAi knockdown of MEK inhibitor-responsive RTKs in SUM159 cells enhances growth inhibition in response to U0126. Knockdown of RTKs was performed in the presence or absence of 5 μM U0126 for 96 h and cell growth determined by Cell-Titer Glo. (FIG. 5C) Cotreatment with AZD6244 and sorafenib synergizes in cell growth inhibition of SUM159 cells. SUM159 cells were treated with DMSO, sorafenib, AZD6244 or the combination of AZD6244 and sorafenib and cell growth determined by Cell-Titer Glo. (FIG. 5D) Treatment of SUM159 cells with the combination of AZD6244 and foretinib enhance growth inhibition. SUM159 cells were treated with DMSO, foretinib, AZD6244 or the combination of AZD6244 and foretinib and cell growth determined by Cell-Titer Glo. (FIG. 5E) The combination of AZD6244 and sorafenib inhibit cell growth greater than AZD6244 treatment alone. SUM159 cells were treated with DMSO, sorafenib, AZD6244 or the combination of AZD6244 and sorafenib and cell growth determined by cell counting. (FIG. 5F) Sorafenib inhibits AZD6244-mediated activation of RTKs. SUM159 cells were treated with DMSO, sorafenib, AZD6244 or the combination of AZD6244 and sorafenib for 72 h and RTK tyrosine phosphorylation determined by RTK arrays. (FIG. 5G) Combined treatment of SUM159 cells with AZD6244 and sorafenib enhances inhibition of ERK activity and primes cells for apoptosis. SUM159 cells were treated with DMSO, sorafenib, AZD6244 or the combination of AZD6244 and sorafenib for 72 h and BIM, cyclin D1 expression and ERK activity determined by western blot. (FIG. 5H) AZD6244 activation of ERK1/2 requires RTK and MEK activity. Inhibition of ERK activity in AZD6244-resistant SUM159 cells occurs in response to high dose (50 μM) AZD6244 treatment or cotreatment of low dose (5 μM) AZD6244 and 250 nM sorafenib. (FIG. 5I) SUM159-R cells require AZD6244-induced RTK activity for drug resistance Inhibited cell growth in SUM159-R cells treated with 250 nM sorafenib for 72 h determined by cell counts.*p-value <0.001; Error bars represent triplicate experiments ±S.D.

FIG. 6A-6F. AZD6244-mediated kinome reprogramming in C3Tag mouse model of TNBC. (FIG. 6A) Induction of PDGFRβ in AZD6244 (20 mg/kg) treated C3-Tag tumors determined by anti-PDGFRβ immunofluorescence. (FIG. 6B) Tumor-derived C3Tag cell line show AZD6244-mediated c-Myc loss and induction of RTKs. C3Tag cell line treated with 5 μM AZD6244 for 4 and 24 h and RTK reprogramming determined by western blot. (FIG. 6C) AZD6244-dependent RTK reprogramming in C3Tag GEMM is inhibitor-specific. Induction of RTKs in C3Tag tumors treated with AZD6244 (20 mg/kg) for 28 days compared to sorafenib (30 mg/kg) treatment for 26 days relative to untreated C3Tag tumors determined by western blot. (FIG. 6D) AZD6244-mediated c-Myc degradation induces PDGFRβ expression in C3Tag GEMM. c-Myc protein levels were determined for C3Tag tumors treated with AZD6244 (20 mg/kg) for 2 and 7 days. (FIG. 6E) AZD6244 kinome reprogramming is distinct from sorafenib response. MIB/MS profile of C3Tag tumors in response to AZD6244 (20 mg/kg) for 28 days or sorafenib (30 mg/kg) for 26 days. The line graphs show iTRAQ determined quantitative changes in MIB binding as a ratio of Inhibitor/untreated. Ratio <1 denotes decreased MIB binding and >1 increased MIB binding of kinases in treated versus control tumors. (FIG. 6F) MEK2 escapes AZD6244 inhibition while MEK1 remains inhibited. MIB/MS profile of MEK-ERK binding from C3Tag mice treated with AZD6244 (20 mg/kg) for 28 days relative to untreated.

FIG. 7A-7G. Combination of AZD6244 and sorafenib causes apoptosis and tumor regression in C3Tag TNBC mouse model. (FIG. 7A) AZD6244 or sorafenib fed in chow results in ERK activation after 2 and 7 days of treatment in C3Tag GEMM. Combination of AZD6244 and sorafenib, but not single agents, inhibits RTK-mediated ERK activation. RTK reprogramming following MEK inhibition was monitored in tumors treated with AZD6244 (20 mg/kg) and sorafenib (30 mg/kg), alone or in combination, relative to untreated tumors by western blot. (FIG. 7B) The combination of AZD6244 and sorafenib enhances c-Myc degradation in C3Tag tumors. c-Myc levels were determined in tumors treated with AZD6244 (20 mg/kg) and sorafenib (30 mg/kg), alone or in combination for 2 days relative to untreated by western blot. (FIG. 7C) Combining AZD6244 and sorafenib inhibits PDGFRβ (Y751) phosphorylation, reduces cyclin D1 levels and increases BIM expression in tumors of C3Tag mice. The effect of sorafenib on AZD6244-dependent survival was determined by western blot for tumors treated with AZD6244 (20 mg/kg) and sorafenib (30 mg/kg), alone or in combination, relative to untreated. (FIG. 7D) Sorafenib inhibits AZD6244-dependent activation of ERK, promoting c-Myc degradation and loss of cyclin B1 expression in C3Tag tumor-derived cell line. C3Tag cell line was treated with DMSO, AZD6244, sorafenib or the combination of AZD6244 and sorafenib and ERK activation determined by western blot. (FIG. 7E) AZD6244 and sorafenib synergize to inhibit cell growth in C3Tag cell line. C3Tag cells were treated with DMSO, AZD6244, sorafenib or the combination of AZD6244 and sorafenib and cell growth determined by cell counts (*p-value <0.001; quadruplicate experiments). (FIG. 7F) Combined treatment of C3Tag mice with AZD6244 and sorafenib for 21 days causes significant tumor regression compared to AZD6244 alone. C3Tag mice were treated with AZD6244 (20 mg/kg), sorafenib (30 mg/kg) or the combination of AZD6244 and sorafenib and compared to untreated tumors. Percent change in tumor volume of drug treated relative to untreated is shown (*Wilcoxon p-value 0.007). (FIG. 7G) Increased apoptosis of C3Tag mouse tumors following combination treatment of AZD6244 and sorafenib. Apoptosis in C3Tag tumors treated for 2 days with AZD6244 (20 mg/kg) and sorafenib (30 mg/kg), alone or in combination, relative to untreated was determined by TUNEL and DAPI staining.

FIG. 8A-8E. Activation-dependent binding of kinases to Multiplexed Inhibitor Beads (MIBs). (FIG. 8A) Structures of kinase inhibitors conjugated to beads used as Multiplex Inhibitor Beads. See Section 5.4 and 6.16 for additional structures. (FIG. 8B) Increased binding of EGFR signaling components following EGF stimulation. SILAC labeled MDA-MB-231 cells were serum starved overnight and stimulated with 30 ng/mL EGF for 15 min, harvested and applied to MIBs. A SILAC-based quantitative comparison of MIB-bound kinases from serum starved versus EGF stimulated cells was performed. (FIG. 8C) Increased binding of tyrosine kinases to MIBs following pervanadate phosphatase inhibitor treatment. SILAC labeled chronic myeloid leukemia cells (Myl) were treated with 100 μM pervanadate for 15 min, harvested and kinome isolated using MIBs. A SILAC-based comparison of MIB-bound kinases in the presence or absence of pervanadate was determined. (FIG. 8D) Shows the luminescence change of BT474 cells on treatment with lapatinib. BT474 cells were treated with 50 nM lapatinib over a time course of 72 hours (timepoints are 0, 24, 48, and 72 hrs) and monitored for growth inhibition by luminescent cell viability assays. This figure shows the growth inhibition of BT474 cells by lapatinib. (FIG. 8E) Shows the change in the kinome for a variety of clinically relevant kinases before and after treatment with lapatinib. See the chromatography in Section 6.18 and the mass spectroscopy methods in Section 6.11 for the experimental details. The different colors represent different time points of (50 nM) lapatinib treatment relative to DMSO control-treated cells. From light to dark, the time points are 4, 24, and 48 hrs of lapatinib treatment.

FIG. 9A-9F. Kinome profile of MDA-MB-231 TNBC cells in response to MEK1/2 inhibition. (FIG. 9A) MDA-MB-231 cells are growth inhibited by 5 μM U0126 or AZD6244. Triplicate experiments+SD. (FIG. 9B) Inhibition of ERK1/2 activity in MDA-MB-231 cells in response to 5 μM U0126 or AZD6244 treatment for 4 or 24 h. (FIG. 9C) Kinase phosphorylation is altered in response to AZD6244 treatment. SILAC-labeled SUM159 cells were treated with 5 μM AZD6244 and changes in pSer, pThr, and pTyr phosphopeptides were identified by TiO2 enrichment of MIB elutions. (FIG. 9D) Induction of PDGFRβ in response to MEK1/2 inhibition in MDA-MB-231 cells. SILAC labeled cells were treated with 5 μM U0126 or AZD6244 for 24 h, harvested and kinases isolated using MIBs. Quantitative comparison of MIB-bound kinases treated with MEK1/2 inhibitors compared to DMSO-treated cells. (FIG. 9E) Increased RTK activity in claudin-low cell lines following inhibition of MEK1/2. SUM159 and MDA-MB-231 cells were treated with 5 μM U0126, AZD6244 or DMSO for 24 h and analyzed using RTK arrays. (FIG. 9F) Top 40 kinases expressed in patient claudin-low tumor. RPKM values for each kinase determined by RNA-seq (* denotes AZD6244-responsive kinase in SUM159/MDA-MB-231 cell profiling).

FIG. 10A-10G. Transcriptional upregulation of RTKs and cytokines in MDA-MB-231 cells and specificity of kinome response. (FIG. 10A) MDA-MB-231 cell response to AZD6244 parallels SUM159 response. (FIG. 10B) Transcriptional upregulation of kinases following 5 μM AZD6244 treatment in MDA-MB-231 cells, as determined by qRT-PCR. (FIG. 10C) Transcriptional upregulation of cytokines following 5 μM AZD6244 treatment in MDA-MB-231 cells, as determined by qRT-PCR. (FIG. 10D) Return of cyclin expression in SUM159-R cells compared to SUM159 cells treated with AZD6244, as determined by qRT-PCR. (FIG. 10E) SUM159 cells are growth inhibited by U0126 (5 μM), AZD6244 (5 μM), and BEZ235 (50 nM). (FIG. 10F) BEZ235 inhibits p70 S6 kinase activity but not ERK1/2 signaling in SUM159 cells. (FIG. 10G) Treatment of SUM159 cells with BEZ235 (50 nM) induces a distinct kinome response compared to AZD6244 or U0126 (5 μM), as determined by MIBs/MS using iTRAQ. Drug treatments are standardized to untreated SUM159 cells, and only kinases with statistically significant changes (p<0.1) are shown. Error bars represent triplicate experiments+SD.

FIG. 11A-11F. c-Myc loss causes induction of RTKs. (FIG. 11A) ChIP-PCR with c-Myc antibody shows enrichment for c-Myc at PDGFRβ promoter in SUM159 cells. (FIG. 11B) siRNA-mediated knockdown of both ERK1 and ERK2 suppresses c-Myc expression and causes induction of PDGFRβ transcript, as determined by qRT-PCR. (FIG. 11C) PI3K/mTOR inhibition by BEZ235 (100 nM) does not affect c-Myc protein levels, as determined by western blot. (FIG. 11D) Washout of AZD6244 from SUM159-R causes reduction in RTK and cytokine transcript, shown by qRT-PCR. (FIG. 11E) qRT-PCR of SUM159 cells treated with AZD6244 (5 μM) and/or bortezomib (10 nM). Inhibition of the proteasome with bortezomib prevents AZD6244-mediated loss of c-Myc transcript. (FIG. 11F) Treatment of SUM159-R cells with bortezomib (10 nM) stabilizes c-Myc transcript levels to a level near that of wildtype. Error bars represent triplicate experiments+SD.

FIG. 12A-12H. Combination of AZD6244 with tyrosine kinase inhibitors in SUM159 and MDA-MB-231 cells. (FIG. 12A) Growth inhibition and synthetic lethal-like responses of siRNA-mediated knockdown of MAPK components or PI3K/Akt in SUM159 cells in the presence of U0126. (FIG. 12B) Synthetic lethal-like responses in MDA-MB-231 upon knockdown of induced RTKs in the presence of U0126. Lyn and EphA2 are negative controls not induced by U0126. (FIG. 12C) AZD6244 synergizes with sorafenib to inhibit MDA-MB-231 cell growth. (FIG. 12D) Combination of AZD6244 and foretinib has little effect on MDA-MB-231 cell growth. (FIG. 12E) Cotreatment of MDA-MB-231 cells with AZD6244 and sorafenib for 72 h inhibits AZD6244-mediated tyrosine phosphorylation of PDGFRβ. (FIG. 12F) Cotreatment of SUM159 cells with AZD6244 and sorafenib for 72 h synergizes to inhibit cell growth better than the combined treatment with AZD6244 and targeted RAF inhibitors PLX4720 or SB590885. (FIG. 12G) Cotreatment of SUM159 cells with AZD6244 and sorafenib for 72 h synergizes to inhibit cell growth better than the combined treatment with AZD6244 and targeted RAF inhibitors PLX4720 or SB590885. (FIG. 12H) Western blotting shows enhanced BIM expression and cyclin D1 loss with combined AZD6244/sorafenib treatment for 72 h in MDA-MB-231 cells. Error bars represent triplicate experiments+SD.

FIG. 13. AZD6244 kinome response signature in C3Tag GEMM. (FIG. 13) Control and induction of PDGFRβ expression in tumors from two independent C3Tag mice following treatment with 20 mg/kg AZD6244 shown by immunofluorescence. Representative fields of tumors shown.

FIG. 14A-14B. Combined treatment of AZD6244 and sorafenib synergize to promote apoptosis in C3Tag GEMM. (FIG. 14A) Tumor volumes of C3Tag breast tumors during a 21 day time course of AZD6244 and/or sorafenib treatment. (FIG. 14B) Control. Increased apoptosis in tumors from three distinct C3Tag mice following sorafenib, AZD6244, or combination treatment with AZD6244 and sorafenib compared to single agent treatment or no treatment, as shown by TUNEL and DAPI staining. Representative fields of tumors shown.

FIGS. 15A and 15B shows the pairwise plot of the log 2 protein ratio (FIG. 15B, negative log p-values) for the replicates and the pooled data.

FIG. 16 shows that the overlap/concordance between the individual replicates is greater than 70% among the top 20 kinases.

FIG. 17 displays the Venn diagram of the overlaps between these lists of significant kinases.

FIG. 18 shows the changes in the kinome for a head and neck cell line before and after treatment with rapamycin.

FIG. 19 shows the changes in the kinome for a leukemia cell line before and after treatment with a MERTK inhibitor.

FIG. 20 shows the changes in the kinome for human fibroblast cells before and after CMV infection.

### 5. DETAILED DESCRIPTION OF THE INVENTION

### 5.1. Definitions

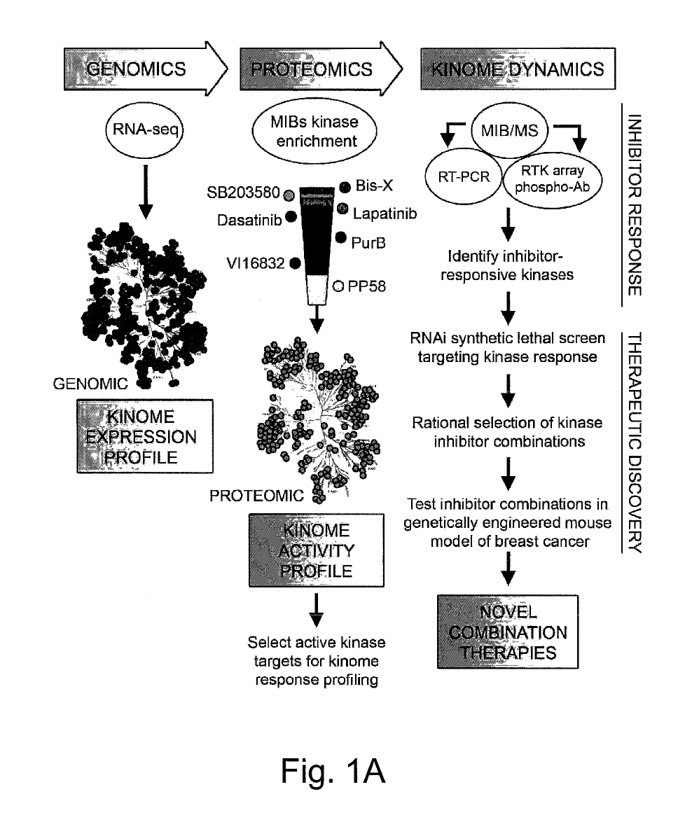

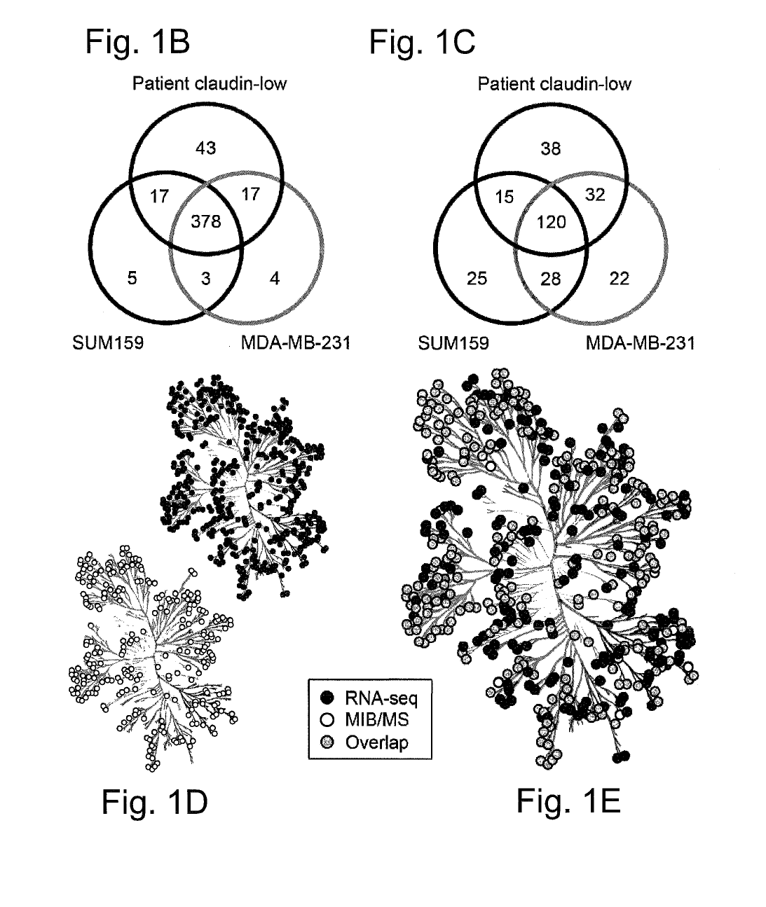

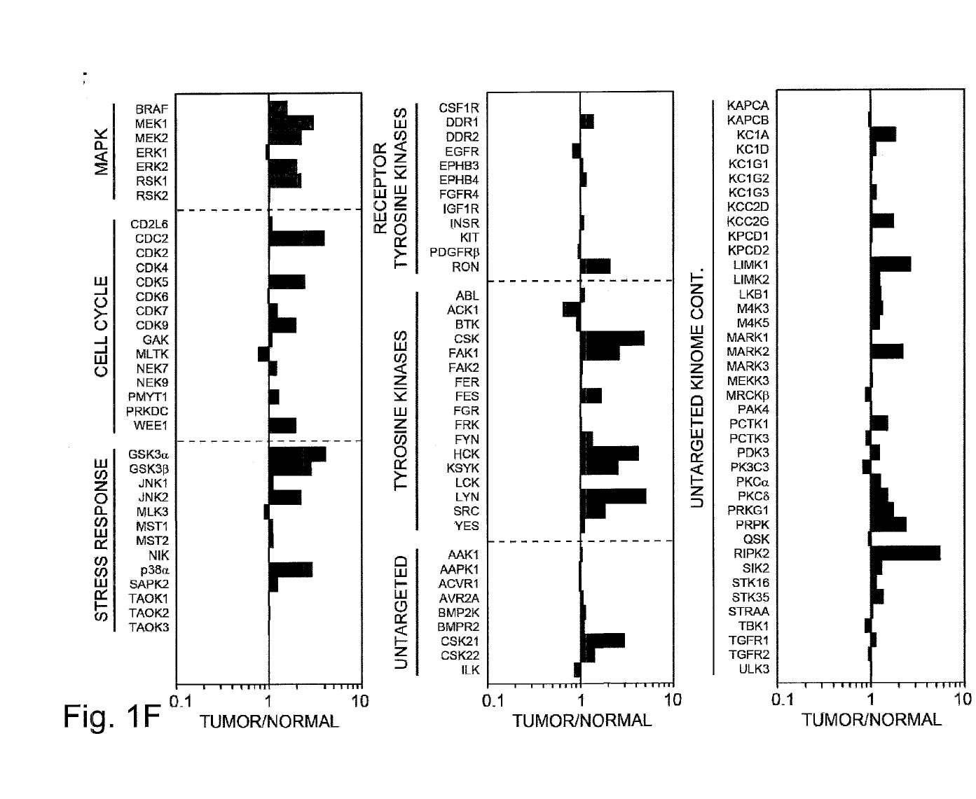

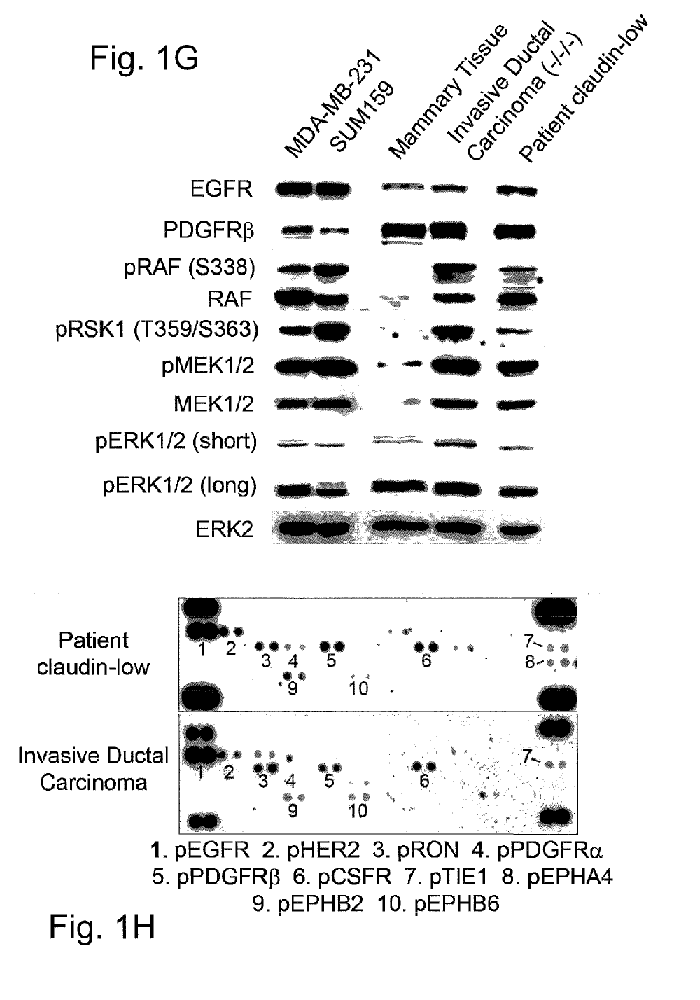

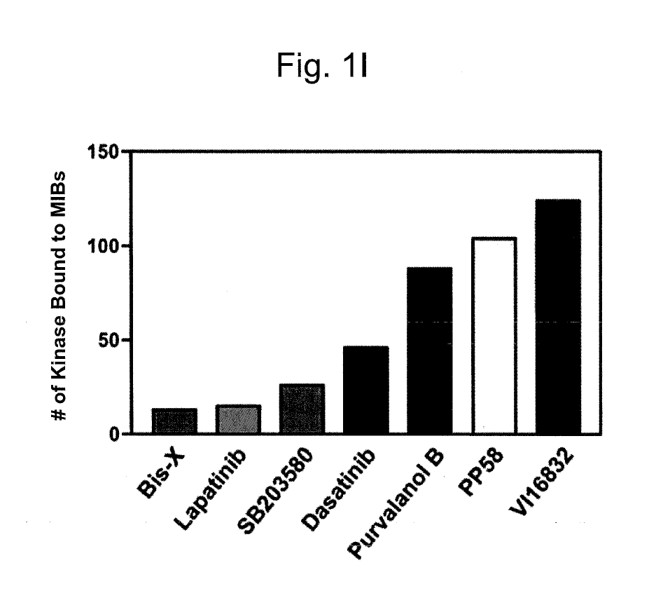

As used herein the term “specific binding affinity” means and includes a ligand that binds to 20 or fewer kinases by profiling individual inhibitor beads using 5 mg of cell lysate protein. See also Section 6.18 below and FIG. 1(I). Bis-X, lapatinib and AZD6244 have specific binding affinity. Moreover, lapatinib and AZD6244 are type II kinase inhibitors that do not bind in the generic activated state of the ATP binding site which increases their selectivity.

As used herein the term “non-specific binding affinity” means and includes a ligand that binds to 50 or more kinases determined by profiling individual inhibitor beads using 5 mg of cell lysate protein. See also Section 5.18 below and FIG. 1(I). Another way to describe ligands that have non-specific binding affinity in the MIBs is that the 2,4-diaminopyrimidine, pyrazole ligands, PP58 and VI16832 are pan-kinase inhibitors that bind a number of families of the kinome. Furthermore, PP58 and VI16832 were engineered to bind multiple kinases and to not be specific for a given kinase.

As used herein the term “solid support” means and includes any support capable of binding the affinity ligands disclosed herein. Well-known supports or carriers include glass, polystyrene, polypropylene, polyethylene, dextran, nylon, amylases, natural and modified celluloses, polyacrylamides, and magnetite. The support material may have virtually any possible structural configuration so long as the coupled affinity ligand is capable of binding to kinases. Thus, the support configuration may be spherical, as in a bead, or cylindrical, as in the inside surface of a test tube, or the external surface of a rod. Alternatively, the surface may be flat such as a sheet, test strip, etc. In one non-limiting embodiment, the solid support may be sepharose or polystyrene beads. Those skilled in the art will know many other suitable carriers for binding antibody or antigen, or will be able to ascertain the same by use of routine experimentation.

As used herein, “clinical signs of cancer” means and includes any sign or indication of the existence of cancer in a subject, which sign or indication would be well known to the skilled artisan (e.g., oncologist, nurse practitioner). The clinical signs of cancer may be any symptom known to be associated with the cancer. Clinical signs of some cancers include, for example, chronic pain, nausea, vomiting, abnormal taste sensation, constipation, urinary symptoms (e.g., bladder spasm), respiratory symptoms, skin problems (e.g., pruritus, hair loss), or fever, among others.

As used herein, “remission” means and includes a period during which the symptoms of a cancer have been reduced or eliminated, as remission is ordinarily defined in the oncology art.

As used herein “serially monitoring” levels of kinases in a sample, refers to measuring levels of kinases in a sample more than once, e.g., quarterly, bimonthly, monthly, biweekly, weekly, every three days, daily, or several times per day. Serial monitoring of a level includes periodically measuring levels of kinases at regular intervals as deemed necessary by the skilled artisan.

The term “standard level” as used herein refers to a baseline level of a kinase as determined in one or more normal subjects. For example, a baseline may be obtained from at least one subject and preferably is obtained from an average of subjects (e.g., n=2 to 100 or more), wherein the subject or subjects have no prior history of cancer. In the present invention, the measurement of kinase levels may be carried out using the multiplexed inhibitor beads as described.

As used herein, “elevation” of a measured level of a kinase relative to a standard level means that the amount or concentration of a kinase in a sample is sufficiently greater in a subject relative to the standard to be detected by the methods described herein. For example, elevation of the measured level relative to a standard level may be any statistically significant elevation which is detectable. Such an elevation may include, but is not limited to, about a 1%, about a 10%, about a 20%, about a 40%, about an 80%, about a 2-fold, about a 4-fold, about an 8-fold, about a 20-fold, or about a 100-fold elevation, or more, relative to the standard. The term “about” as used herein, refers to a numerical value plus or minus 10% of the numerical value.

As used herein, reference to “measuring a level of a plurality of kinases” in a method of the invention means measuring the level of two or more kinases. In some embodiments, the level and phosphorylation state of 50, 100, 150, 200, 250, or more kinases are measured simultaneously. As used herein, an affinity ligand with which the amount or concentration of a kinase may be determined, includes but is not limited to small molecules. Such small molecules would be modified to have a suitable linker to a bead or other solid support. One of skill in the art would know how to use kinase crystal structures and other publically available documents to design such linkers so the linker does not interfere with the ligand binding to the kinase. Examples of small molecule structures that would be modified to serve as a ligand are commercially available kinase inhibitors such as: ABT-737[852808-04-9], ABT-869[796967-16-3], AC-220 [950769-58-1], Adaphostin, AEE-788 (NVP AEE-788), AEW-541 (NVP AEW-541), Afatinib (BIBW2992), AG1296, AG13958[319460-94-1], AG1478, AG-490, Akt-I-1,2[473382-48-8], Akt-1-1 [473382-39-7], AMG-47a [882663-88-9], AMG479, AMG-Tie2-1 [870223-96-4], Amuvatinib (MP-470[850879-09-3]), AP23236, AP23464, Apatinib (YN968D1), ARQ-197, AS252424[900515-16-4], AT7519[902135-91-5], AT9283[896466-04-9], AV-412[451492-95-8], AV-951[475108-18-0], Axitinib (AG13736), AZ-960[905586-69-8], AZD7762 [860352-01-8], AZM475271, BEZ235 (NVP-BEZ235), BGT226 (NVP-BGT226), BI2536 (R-)[755038-02-9], BIBW-2992, BI-D1870[501437-28-1], BIRB796, Bis-X (Bisindoylmaleimide-X, RO-31-8425), BKM120 (NVP-BKM120), BLY719, BMS-5, BMS599626 (AC-480), Bosutinib (SKI-606), Brivanib (BMS582664), BX-795[702675-74-9], BX-912[702674-56-4], Cabozantinib (BMS90735′, XL-184), Canertinib (CI1033, PD183805, SN26606), CAL-101 (GS-1101), CC-401[395104-30-0], Cediranib (AZD2171, NSC732208), CEP11981, CEP7055, CGP76030, CGP77675, CI1040[212631-79-3], CL387785, CP358774, CP547632, CP654577, CP690550, CP724714, CP751871, CP868596, Crizotinib (Xalkori®, PF-02341066, 1066), CUDC-101, CUDC-907, CX4945 [1009820-21-6], CYC-116[693228-63-6], CYT11387 [1056634-68-4], Dabrefenib (GSK2118436), Danusertib [827318-97-8], Dasatinib (BMS354825), Deforolimus (AP23575, MK8669), Dovitinib lactate (TKI-258; CHIR-258), E7080[417716-92-8], EKB-569, Enzastaurin (LY317615), Erlotinib (Tarceva®, OSI-774), Everolimus (Affinitor®), EXEL0862, Flavopiridol (alvocidib) [146426-40-6], Foretinib (XL-880; GSK 1363089), Fostamatinib, GDC0941[957054-30-7], Gefitinib (Iressa®, ZD1839), GNE-490 [1033739-92-2], GNE-493 [1033735-94-2], Gö6976, GSK1070916A, GSK690693[937174-76-0], GSK461364, GSK2126458, GTP14564, GW441756[504433-23-2], IC87114[371242-69-2], GW5074, Ibrutinib (AVL-263), Icotinib (BPI-2009H), Imatinib mesylate (Gleevac®, STI571, CGP57148), INCB018424, INK1197, JNJ38877605[943540-75-8], JNJ7706621 [443797-96-4], Ki20227 (+/−) [623142-96-1], KI23819, KRN-633, KRN-951, KU0063794 [938440-64-3], KU55933[587871-26-9], KX2-391, L21649, Lapatinib (Tykerb®, GW-572016), Leflunomide (SU-101), Lestaurtinib (CEP-701), LFM-A13, Linifanib (M10-963; ABT-869), Masatinib (AB-1010) [790299-79-5], Merck-5 [457081-03-7], Midostaurin (CGP-41251, PKC-412), MK-2206 ((8-(4-(1-Aminocyclobutyl)phenyl-9-phenyl[1,2,4]triazolo[3,4f][1,6]-naphthyridin-3(2H)-one dihydrochloride), MK-5108 (VX-689), MKC-1 (Ro317453, R-440), MLN8054, MLN8237, Motesanib diphosphate (AMG-706), MP-412, Neratinib (CPD-820, HKI-272, WAY179272), Nilotinib (Tasigna®, AMN107), Nintedanib (Vargatef™, BIBF-1120) NS-187, NVP-BSK805, ON012380, OSI-817, OSI-906, OSI-930, Pazopanib (Votrient®, GW786034), PD153035, PD158780, PD166326, PD173074, PD173955+56, PD180970, PD325901[391210-10-9], PD332991, PD4217903[956905-27-4], PF431396 [717906-29-1], PF562271[717907-75-0], PHA690509 [492445-28-0], PI-103[371935-74-9], PIK-294 [900185-02-6], PIK-75[372196-67-3], PIK-90[677338-12-4], PKI-166, PLX4720, Ponatinib (AP24534), PP1, PP2, PP58, Purvalanol B, R1487[449808-64-4], sirolimus (Rapamune®, rapamycin), RAF265, Regorafenib (BAY 73-4506), RHO-15[864082-47-3], RWJ-67657 [215303-72-3], SB202190[152121-30-7], SB203580, SB216763[280744-09-4], SB242235[193746-75-7], SB590885, SD-06 [271576-80-8], SD-169[1670-87-7], Saracatinib (AZD0530), Seliciclib (Roscovitine, CYC-202, NSC701554), Selumetinib (AZD6244, ARRY-142886), Semaxinib (SU5416), SMI-4a [438190-29-5], SNS-032 (BMS387032), SNS-314[1057249-41-8], Sorafenib (Nexavar®, BAY 43-9006), SR3677[1072959-67-1], staurosporin, SU10944, SU11333+36, SU11464, SU11657, SU14813, SU5402[215543-92-3], SU5614, SU6656, SU6668 (TSU-68), Sunitinib (Sutent®, SU11248), TAK-165, TAK-715[303162-79-0], Tandutinib (MLN518, CT53518), Telatinib (BAY57-9352), Temsirolimus (CCI-779), TG100435, TG101348, TGX-221[663619-89-4], TKI258 (CHIR-258), Toceranib (SU11654), TOVOK [439081-18-2], Tremetinib (GSK01120201, JTP-74057), Tyrphostin AG1024, Tyrphostin AG957, U0126, Vandetanib (Caprelsa®, AZD-6474, CH-331, ZD6474), Vargatef [656247-17-5], Vatalanib (PTK787, ZK222584, CGP79787), Vemurafenib (Zelboraf®, PLX-4032), VI16832, VX-322, VX-680 [639089-54-6], VX-702[745833-23-2], WHI-P131, WHI-P154, WP1034, XL-281, XL-647, XL-999, YM201636[371942-69-7], or ZK-CDK. The numbers in brackets [ ] refer to the catalog number for the compound available from Synkinase (Melbourne, Australia). For kinase inhibitors and additional signal pathway modulators, see also Broekman et al., 2011, World J Clin Oncol 2(2) 80-93; Gravina et al., 2010, Mol Can 9 305 Nov. 25, 2010; Janne et al., 2009, Nat Rev Drug Disc 8 709-723; Levitzki and Klein, 2010, Mol Aspects Med 31 289-329; Kling, 2010 Nat Biotech 28(12) 1236-1238; Marotta et al. 2011 J Clin Invest 121(7):2723-2735 (The JAK1/2/3 inhibitor from Novartis NVP-BSK805 has recently been shown to be effective in the treatment of triple negative breast cancer); Ott and Adams, 2011, Immunotherapy 3(2) 213-227; Richardson et al., 2011, Brit J Haematol 152(4) 367-379; Ruschak et al., 2011 J Nat Canc Inst 103(13) 1007-1017; Thurn et al., 2011 Fut Oncol 7(2) 263-283; Venugopal and Evans, 2011, Curr Med Chem 18(11) 1658-1671; Whittaker et al., 2010, Oncogene 49 4989-5005; Zhang et al., 2009 Nat Rev Cancer 9 28-39; Zhang, 2011 Nat Med 17(4) 461-470, the contents of which are hereby incorporated by reference in their entireties.

The phrase “functional effects” in the context of assays for testing means compounds that modulate a phenotype or a gene associated with a kinase related disorder either in vitro, in cell culture, in tissue samples, or in vivo. This may also be a chemical or phenotypic effect such as altered kinome profiles in vivo, e.g., changing from a high risk kinome profile to a low risk profile; altered expression of genes associated with a kinase related disorder; altered transcriptional activity of a gene hyper- or hypomethylated in a kinase related disorder; or altered activities or the activation state of proteins having enzymatic activities and the downstream effects of proteins encoded by these genes. A functional effect may include transcriptional activation or repression, the ability of cells to proliferate, expression in cells during a kinase related disorder progression, and other cellular characteristics. “Functional effects” include in vitro, in vivo, and ex vivo activities. By “determining the functional effect” is meant assaying for a compound that increases or decreases the transcription of genes, the translation of proteins, or the activation state of proteins having enzymatic activity (such as phosphorylation state or kinase activity) that are indirectly or directly associated with a kinase related disorder. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers.

Validation of the functional effect of a compound on a kinase related disorder occurrence or progression can also be performed using assays known to those of skill in the art such as studies using mouse models. The functional effects can be evaluated by many means known to those skilled in the art, e.g., microscopy for quantitative or qualitative measures of alterations in morphological features, measurement of changes in RNA or protein levels for other genes associated with a kinase related disorder, measurement of RNA stability, identification of downstream or reporter gene expression (CAT, luciferase, β-gal, GFP, and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of the expression of genes hyper- or hypomethylated in a kinase related disorder, mutations associated with a kinase related disorder, or the translation proteins encoded thereby. Inhibitors, activators, or modulators also include naturally occurring and synthetic ligands, antagonists, agonists, antibodies, peptides, cyclic peptides, nucleic acids, antisense molecules, ribozymes, shRNAs, RNAi molecules, small organic molecules and the like. Such assays for inhibitors and activators include, e.g., (1)(a) the mRNA expression, or (b) proteins expressed by genes hyper- or hypomethylated in a kinase related disorder in vitro, in cells, or cell extracts; (2) applying putative modulator compounds; and (3) determining the functional effects on activity, as described above.

Assays comprising in vivo measurement of a kinase related disorder; or genes hyper- or hypomethylated in a kinase related disorder are treated with a potential activator, inhibitor, or modulator are compared to control assays without the inhibitor, activator, or modulator to examine the extent of inhibition. Controls (untreated) are assigned a relative activity value of 100% Inhibition of gene expression, protein expression associated with a kinase related disorder is achieved when the activity value relative to the control is about 80%, preferably 50%, more preferably 25-0%. Activation of gene expression, or proteins associated with a kinase related disorder is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide, small organic molecule, polysaccharide, peptide, circular peptide, lipid, fatty acid, shRNA, siRNA, polynucleotide, oligonucleotide, etc., to be tested for the capacity to directly or indirectly modulate a genotype or phenotype associated with a kinase related disorder. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (“HTS”) methods are employed for such an analysis. The compound may be a “small organic molecule” that is an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

In another embodiment, the invention encompasses a method for predicting the development of resistance to a chemotherapy regimen in a subject, which subject has preferably been treated with a chemotherapy regimen, comprising: serially monitoring levels of a plurality of kinase in a sample obtained from the subject during a period of remission; and comparing the levels measured to standard levels, wherein elevation of the measured level relative to the standard level indicates that the subject is at an increased risk for development of resistance to the chemotherapy regimen.

The chemotherapy regimen to which the subject has become resistant may include any chemotherapy treatment known in the art for treatment of cancer, particularly a cancer associated with aberrant expression and/or activity of a kinase, including but not limited to, treatment with chemotherapeutic agents directed at a signaling pathway or pathways.

Non-limiting examples of signaling pathway modulators or chemotherapeutic agents known in the art are 5-fluorouracil; asparaginase; bevacizumab (Avastin®); bleomycin; campathecins; cetuximab (Erbitux®); crizotinib (Xalkori®); cyclophosphamide; cytarabine; dacarbazine; dactinomycin; dasatinib (Sprycel®); daunorubicin; DNA methyltransferase inhibitors (DNMTs) such as azacitidine (Vidaza®) and decitabine; doxorubicin; doxorubicin; epirubicin; erbstatin; erlotinib (Tarceva®); estramustine; etoposide; etoposide; gefitinib (Iressa®), gemcitabine, genistein, histone acetyl transferase inhibitors (HATs); histone deacetyl transferase inhibitors (HDACs) such as belinostat, entinostat (MS-275), panobinostat, PCI-24781, romidepsin (depsipeptide, FK-228), valproic acid, vorinostat (Zolinza®, SAHA) or heat shock protein inhibitors, including HSP90 inhibitors such as alvespimycin (IPI-493), AT13387, AUY922 (resorcinolic isoxazole amide), CNF2024 (BIIB021), HSP990, MPC-3100, retaspimycin (IPI-504), SNX-2112, SNX-5422, STA-9090, tanespimycin (17-AAG; KOS-953), or XL888; herbimycin A; hexamethylmelamine; hedgehog pathway inhibitors such as saridegib (IPL-926), vismodegib (ERIVEDGE™); hydroxyurea, idarubicin, ifosfamide, imatinib (Gleevec®), irinotecan, lapatinib (Tykerb®), lavendustin A, leucovorin, levamisole, mercaptopurine, methotrexate, mitomycin, mitoxantrone, mTOR inhibitors such as everolimus (Afinitor®), sirolimus (Rapamune®), temsirolimus (Torisel®); nilotinib (Tasigna®); nitrosoureas such as carmustine and lomustine; paclitaxel; panitumumab (Vectibix®); pazopanib (Votrient®); pegaptanib (Macugen®); platinum compounds such as carboplatin, cisplatin, oxaplatin; plicamycin; procarbizine; proteasome inhibitors such as bortezomib (Velcade®); ranibizumab (Lucentis®); sorafenib (Nexavar®); sunitinib (Sutent®); taxanes such as docetaxel, paclitaxel, taxol; thioguanine; topotecan; trastuzumab (Herceptin®); tyrosine kinase inhibitors; tyrphostins; vandetanib (Caprelsa®); vemurafenib (Zelboraf®); vinblastine; vinca alkaloids; vincristine; or vinorelbine. In a preferred embodiment, the chemotherapeutic agent is bevacizumab (Avastin®), cetuximab (Erbitux®), crizotinib (Xalkori®), dasatinib (Sprycel®), erlotinib (Tarceva®), everolimus (Afinitor®), gefitinib (Iressa®), imatinib (Gleevec®), lapatinib (Tykerb®), nilotinib (Tasigna®), panitumumab (Vectibix®), pazopanib (Votrient®), sirolimus (Rapamune®), sorafenib (Nexavar®), sunitinib (Sutent®), temsirolimus (Torisel®), trastuzumab (Herceptin®), vandetanib (Caprelsa®), or vemurafenib (Zelboraf®). Further examples of chemotherapeutic agents may be found in standard publications and texts. See e.g., National Comprehensive Cancer Network (NCCN Guideline™) or Manual of Clinical Oncology, Dennis A. Casciato and Barry B. Lowitz, ed., 4th edition, Jul. 15, 2000, Little, Brown and Company, U.S.

The invention further encompasses a method for improving the effectiveness of cancer treatment in a subject with cancer, comprising: treating the subject with a treatment regimen so as to achieve remission; serially monitoring levels of a plurality of kinases in a sample obtained from the subject during a period of remission; and comparing the levels measured to standard levels, wherein elevation of the measured levels of at least one kinase relative to the standard level indicates that the subject is in need of a modified treatment.

### 5.2. Samples

A sample for the methods of the invention encompasses any sample that can be obtained by invasive or non-invasive techniques from a subject. A sample for the purposes of the invention may include but is not limited to, a biological fluid such as serum, plasma, urine, or blood; a tissue sample; or a tissue extract. Such samples may be obtained by any standard method known in the art, e.g., a finger stick blood sample, a buccal swab, a biopsy, a tape strip, etc.

In a preferred non-limiting embodiment, a sample for the methods of the invention is a biopsy sample; a blood or serum sample; or nucleated cells isolated from a blood sample, obtained from the subject. The sample used in accordance with the methods of the invention need not be obtained from the particular tissue from which the tumor originated. Although not intending to be bound by a particular mechanism of action, given that many kinases are ubiquitously expressed, when therapy, e.g., chemotherapy, is targeted to a particular kinase, that kinase would be targeted throughout the body. Therefore, once resistance to a particular kinase inhibitor therapy develops, it may be detectable throughout the body and not just from the particular tissue from which the tumor originated.

The invention encompasses use of any tissue sampling or biopsy technique known in the art for obtaining a sample from a subject with cancer. In some embodiments, when the subject has breast cancer or a history of breast cancer, any method for obtaining breast tissue known to one skilled in the art can be used, including but not limited to, core biopsies and fine-needle aspirations (see, e.g. Lawrence et al., 2001 J Clin Oncol 19 2754-63; Fabian et al., 1993 J Cell. Biochem 17G 153-160; Boerner et al., 1999 Cancer 87(1) 19-24; Rotten et al., 1993 Eur J Obstet Gynecol Reprod Biol 49(3) 175-86; which are incorporated herein by reference in their entirety). In other embodiments, the invention encompasses lavage and nipple aspiration of breast ductal fluids to obtain a breast tissue sample from a subject with cancer. An exemplary method for lavage and nipple aspiration of breast ductal fluids is presented in Klein et al., 2002 Environ Mol Mutagen 39 127-133), which is incorporated herein by reference in its entirety.

In some embodiments, when the subject has colon cancer, any biopsy or tissue sampling technique known in the art, including but not limited to needle aspiration and solid biopsy, are within the scope of the invention. See, e.g., Greenebaum et al., 1984, Am J Clin Pathol 82(5): 559-64; which is incorporated herein by reference in its entirety.

In the case of lung cancer, the invention encompasses the use of any tissue sampling and biopsy methods known in the art, including but not limited to, fine needle aspirations, EUS-guided fine needle aspirations, bronchial biopsy, transesophogeal biopsy, and broncholaveolar lavage. See, e.g., Devereaux et al., 2002, Gastorintest. Endosc. 56: 397-401; Rosell et al., 1998, Eur. Respir. J. 12(6): 1415-8; Hunerbein et al., 1998, J. Thorac. Cardiovasc. Surge. 116(4): 554-9; Kvale, 1996, Chest Surg. Clin. N. Am. 6: 205-22, all of which are incorporated herein by reference in their entirety. In other embodiments, when the subject has prostate cancer or a history of prostate cancer, any biopsy technique known in the art, including but not limited to needle biopsy and transrectal aspiration biopsy, can be used in the methods of the invention. See, e.g., Kaufman et al., 1982, Urology 19(6): 587-91, which is incorporated herein by reference in its entirety.

### 5.3. Compositions

In non-limiting embodiments, the present invention provides a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity. The first solid support may have specific binding affinity for one or more tyrosine kinases or one or more serine/threonine kinases.

The specific binding affinities may be for kinases selected from the group consisting of Abl, ATK, BRAF, c-KIT, COT, EGFR, FLT-3, HER1, HER2, HER3, HER4, IGF-1R, INSR LYN, MEK, MET, P38, PDGFRβ, PKC/GSK3β, Src, and VEGFR. In one embodiment, the specific binding affinities are for kinases selected from the group consisting of Abl, EGFR, HER2, LYN, P38, and PKC/GSK3β.

Each affinity ligand of the first solid support may binds 20 or fewer kinases and the affinity ligands may be selected from the group consisting of a bisindoylmaleimide-X ligand, a GW-572016 ligand, and a SB203580 ligand.

The non-specific binders on the second solid support may bind ALK, EML-ALK, FGFR1, and FGFR2

Each affinity ligand of the second solid support may bind 50 or more kinases and may be selected from the group consisting of a 2,4-diaminopyrimidine, pyrazole ligand, PP58 ligand, purvalanol B ligand, and a VI16832 ligand.

Further the affinity ligands are designed so as to bine activated mutants of B-Raf, EGFR, MEK, or more generally, kinase fusions and/or activating mutations.

In some embodiments of the multi-analyte column, the specific binding affinity kinase solid supports and the non-specific binding kinase solid supports are present in a molar ratio of ranging from about 4:1 to about 1:4, or from about 1.5:1 to about 1:1.5.

In some embodiments of the multi-analyte column, the first solid support comprises at least three different affinity ligands having specific kinase binding affinity. In other embodiments, the second solid support comprises at least three different affinity ligands having non-specific kinase binding affinity. In still other embodiments the first solid support comprises at least three different affinity ligands having specific kinase binding affinity and the second solid support comprises at least three different affinity ligands having non-specific kinase binding affinity.

### 5.4. Affinity Ligand Structures

In particular non-limiting embodiments, the invention provides compounds having the following structures:

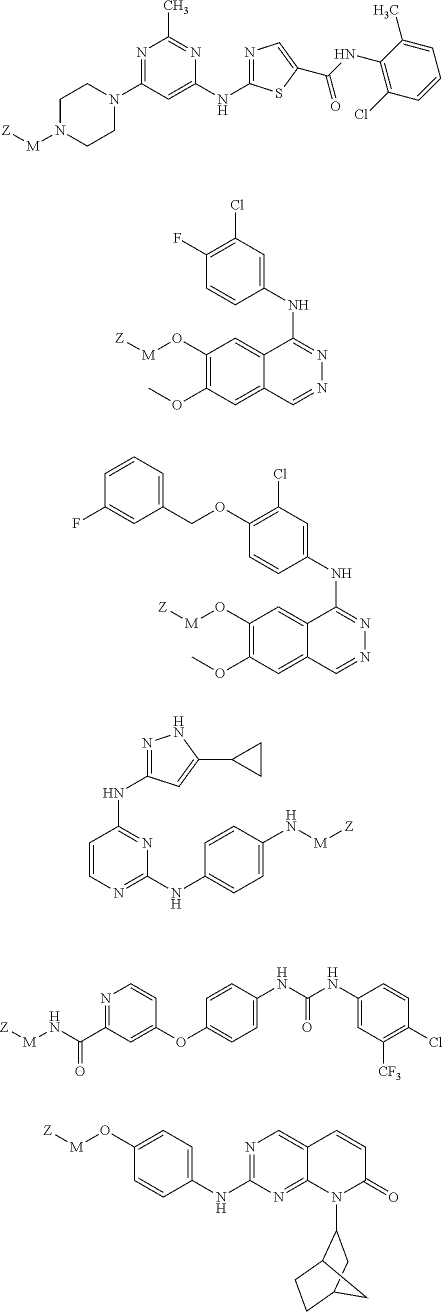

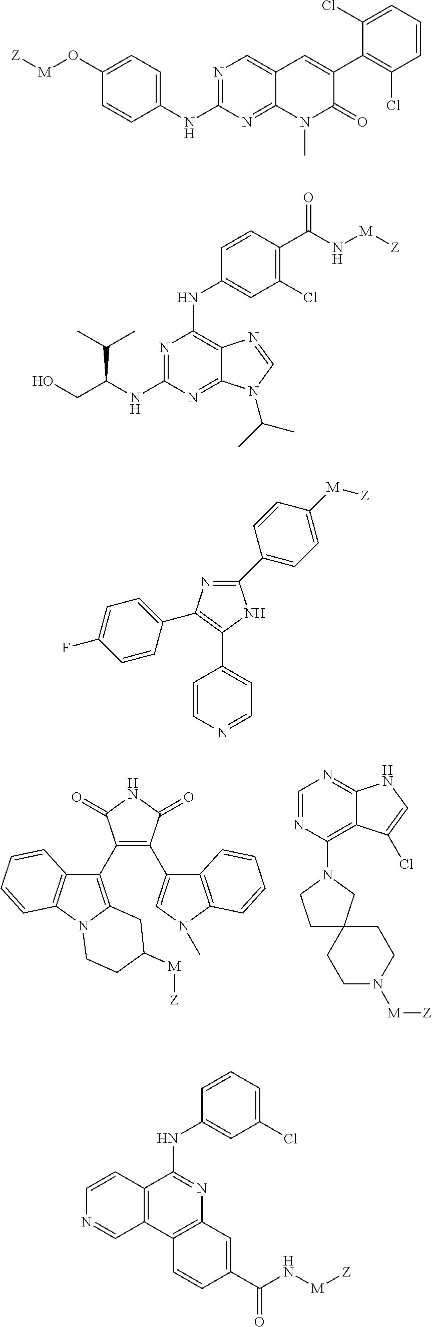

wherein
M is (CH₂)ₓ, (CH₂CH₂OCH₂CH₂)_{y},
Z is I, Br, Cl, F, CN, OH, NO₂, N₃, NH₂, NHR¹, NR²R³, SH, CONH₂, CONHR⁴, CO₂R⁵, CONHNH₂, W
R¹, R², R³, R⁴, and R⁵ are independently selected from hydrogen or C₁₋₈ alkyl or cycloalkyl,
X is 1-16,
Y is 1-12,
W is

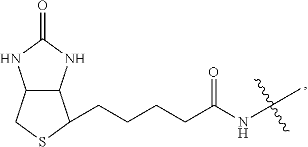 [SMILES: *NC(=O)CCCCC1SCC2NC(=O)NC21]

poly-His tag, or other linkers described in Section 5.5.
In some embodiments,
M is (CH₂)ₓ, (CH₂CH₂OCH₂CH₂)_{y},
Z is I, Br, Cl, OH, N₃, NH₂, NHR¹, CONHNH₂, W,
R¹ is C₁₋₈ alkyl or cycloalkyl,
X is 1-16,
Y is 1-12,
W is

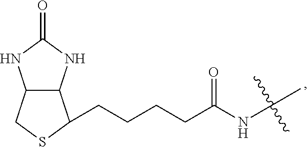 [SMILES: *NC(=O)CCCCC1SCC2NC(=O)NC21]

poly-His tag, or other linkers described in Section 5.5.
In other embodiments,
M is (CH₂)ₓ, (CH₂CH₂OCH₂CH₂)_{y},
Z is COOH, NH₂, or W
X is 1-16,
Y is 1-12,
W is

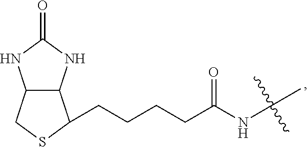 [SMILES: *NC(=O)CCCCC1SCC2NC(=O)NC21]

poly-His tag, or other linkers described in Section 5.5.
In other embodiments, the affinity ligand has a structure of one of the ligands shown in FIG. 8A.
In other embodiments, the affinity ligand has the structure

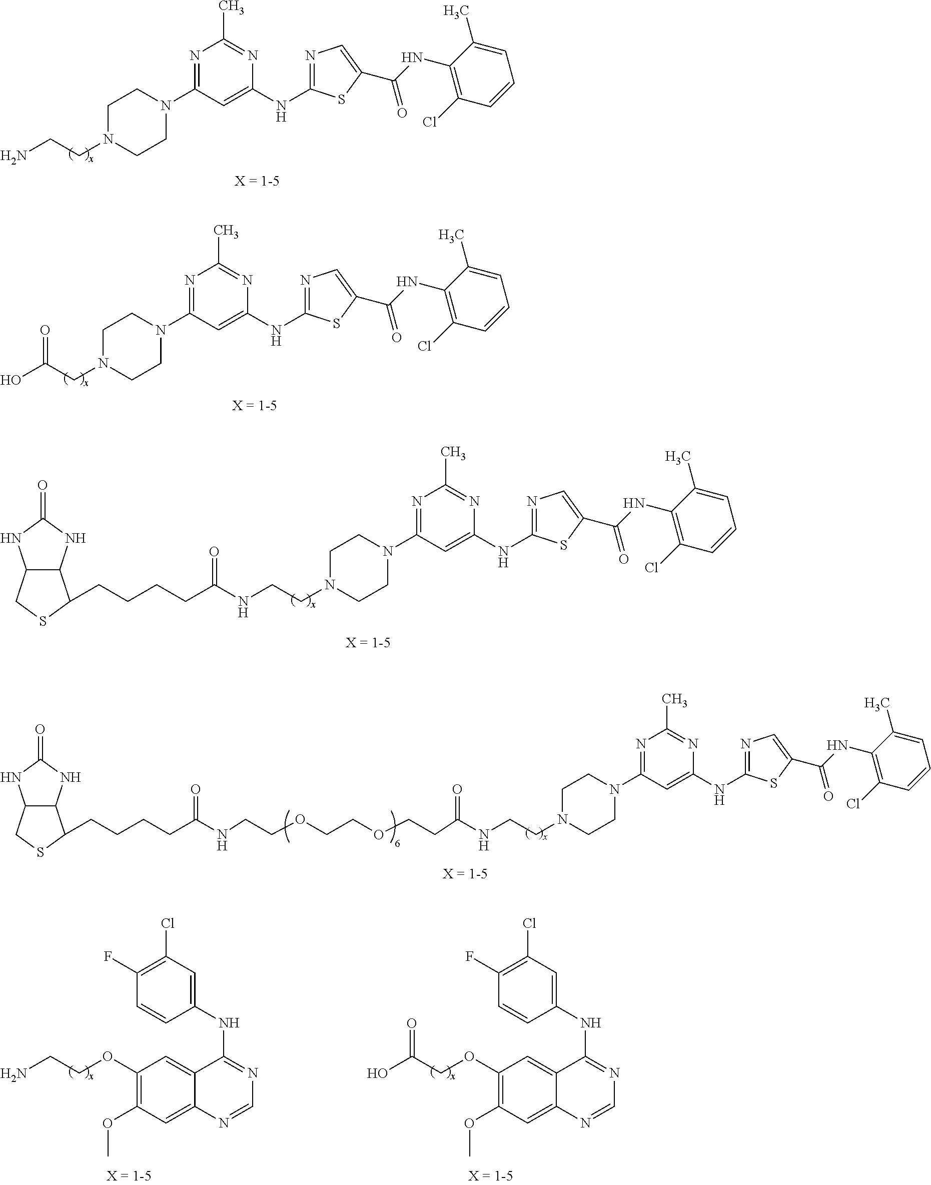

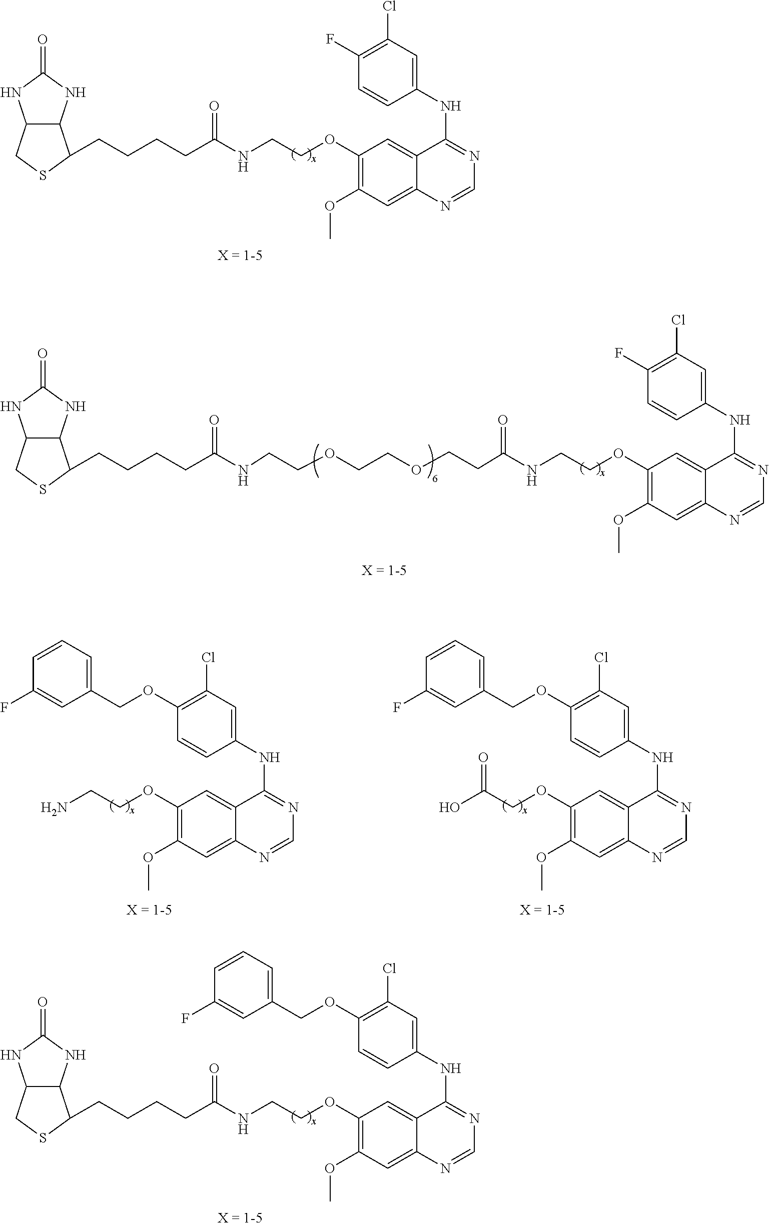

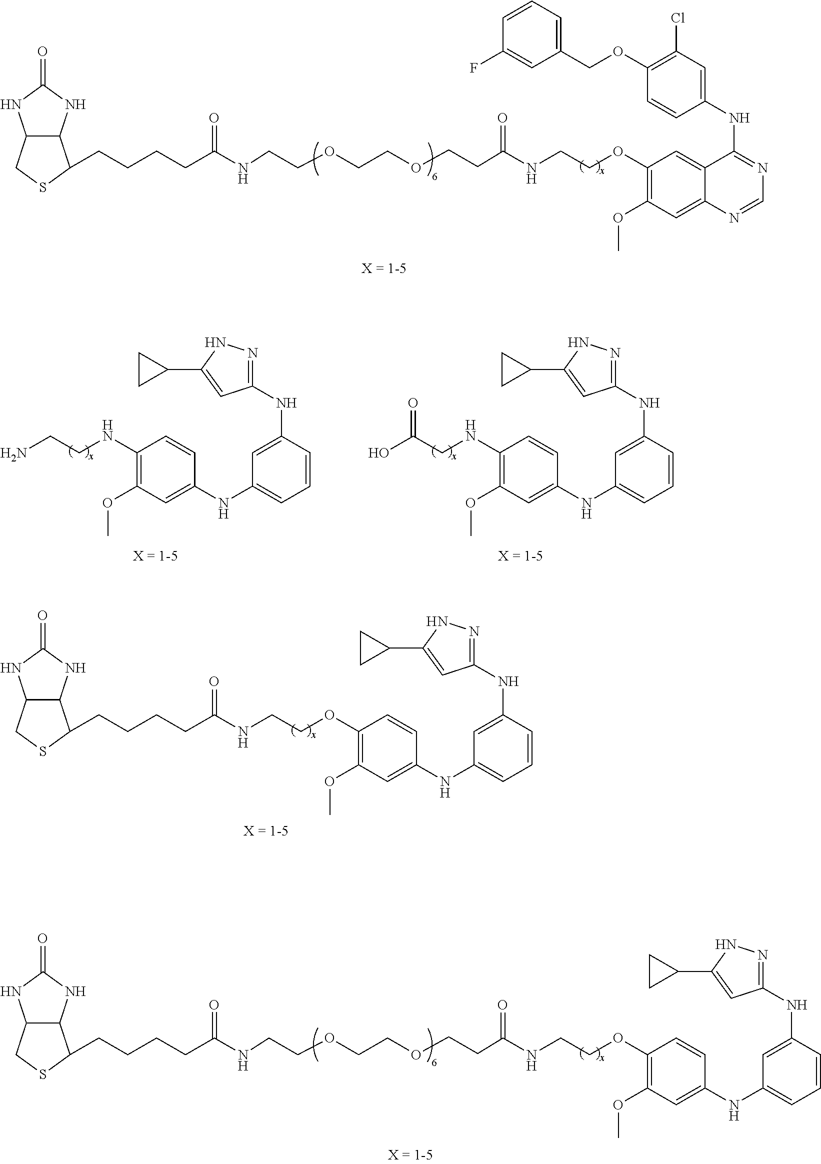

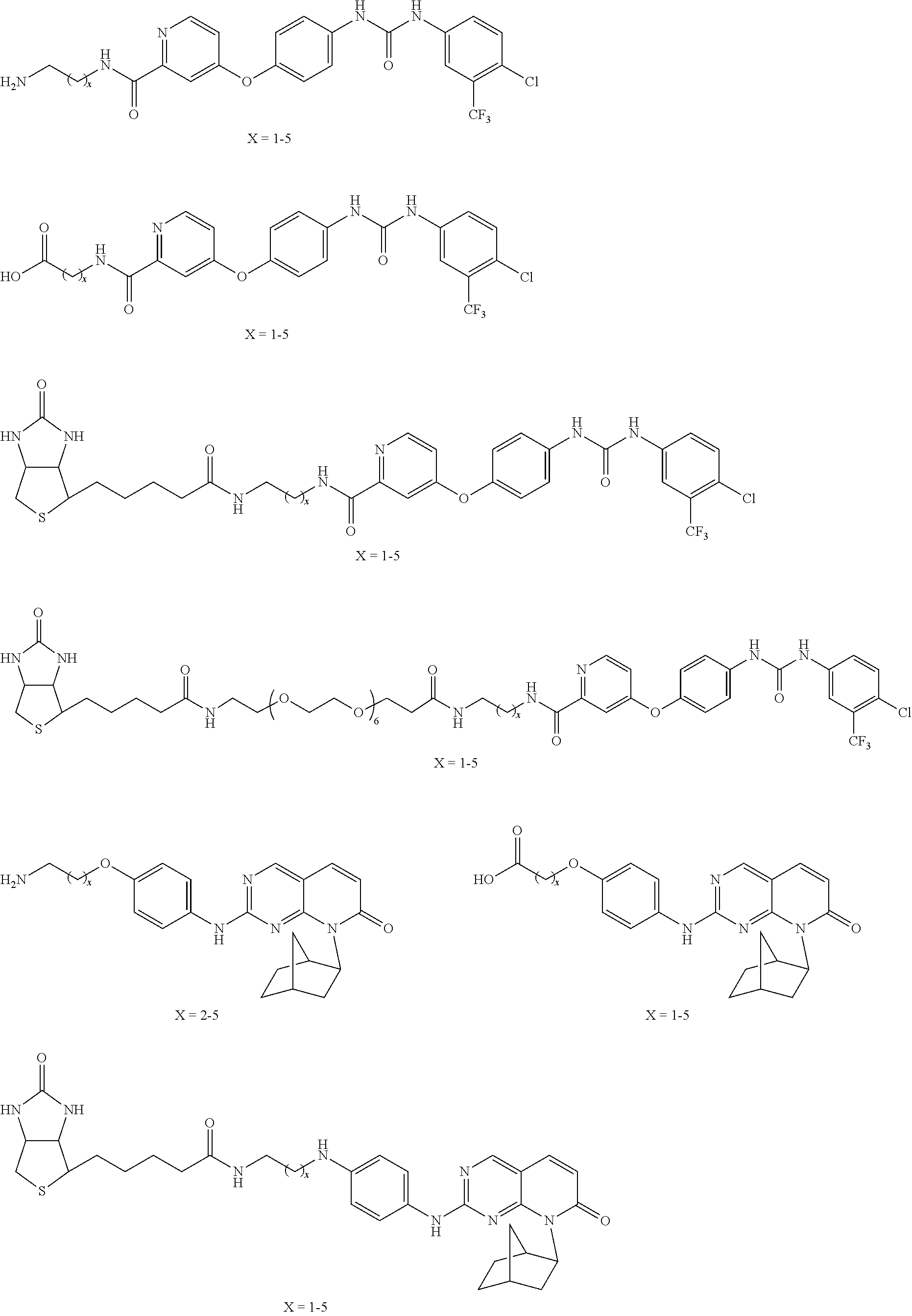

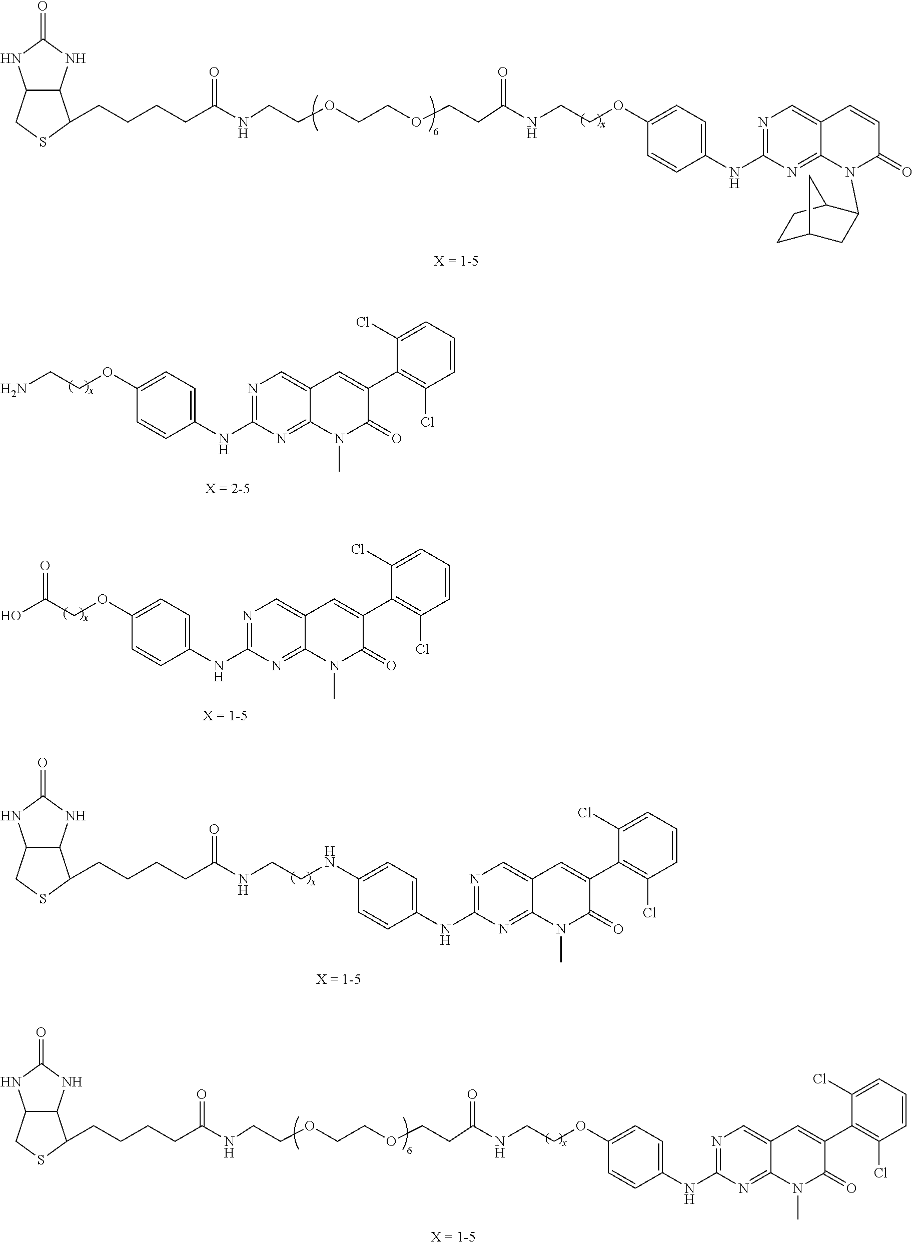

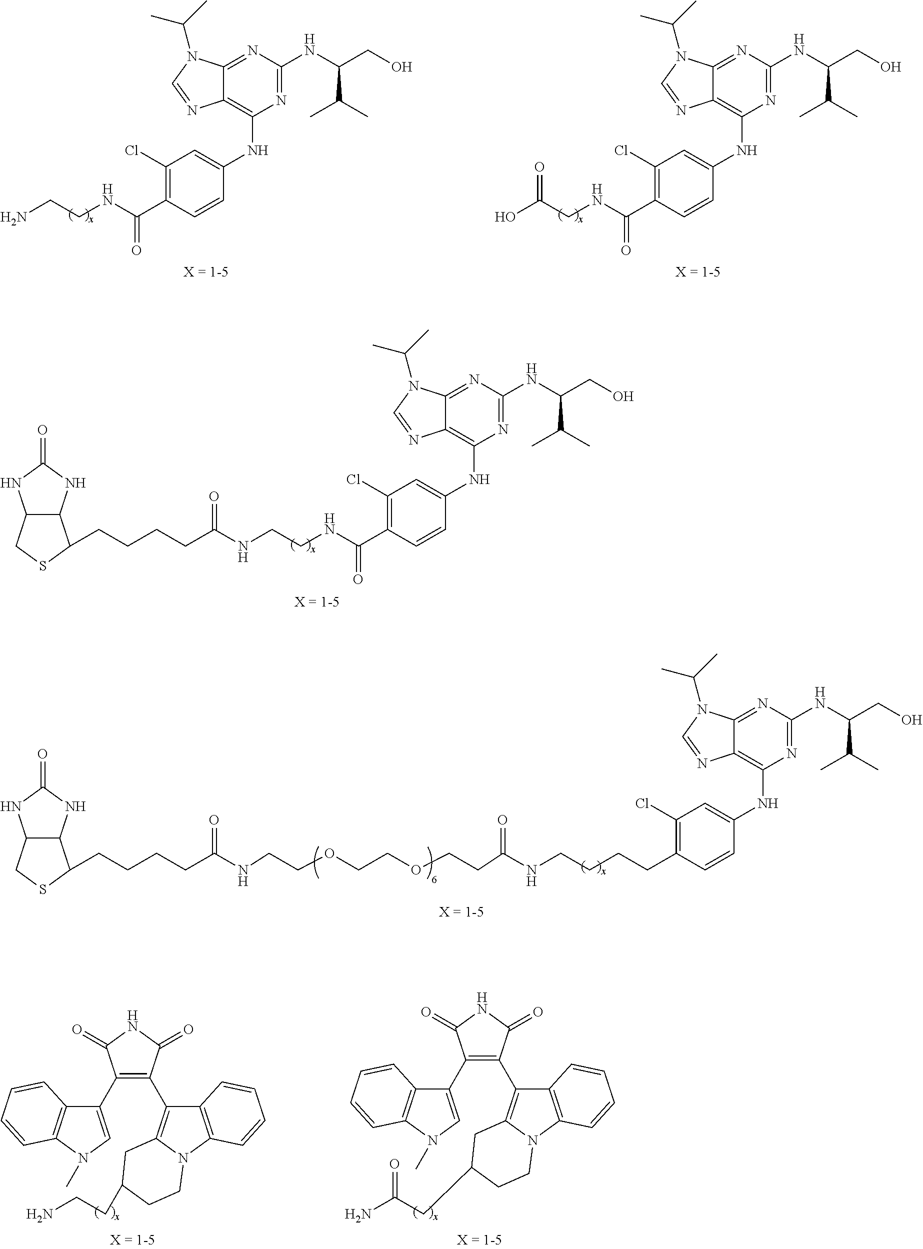

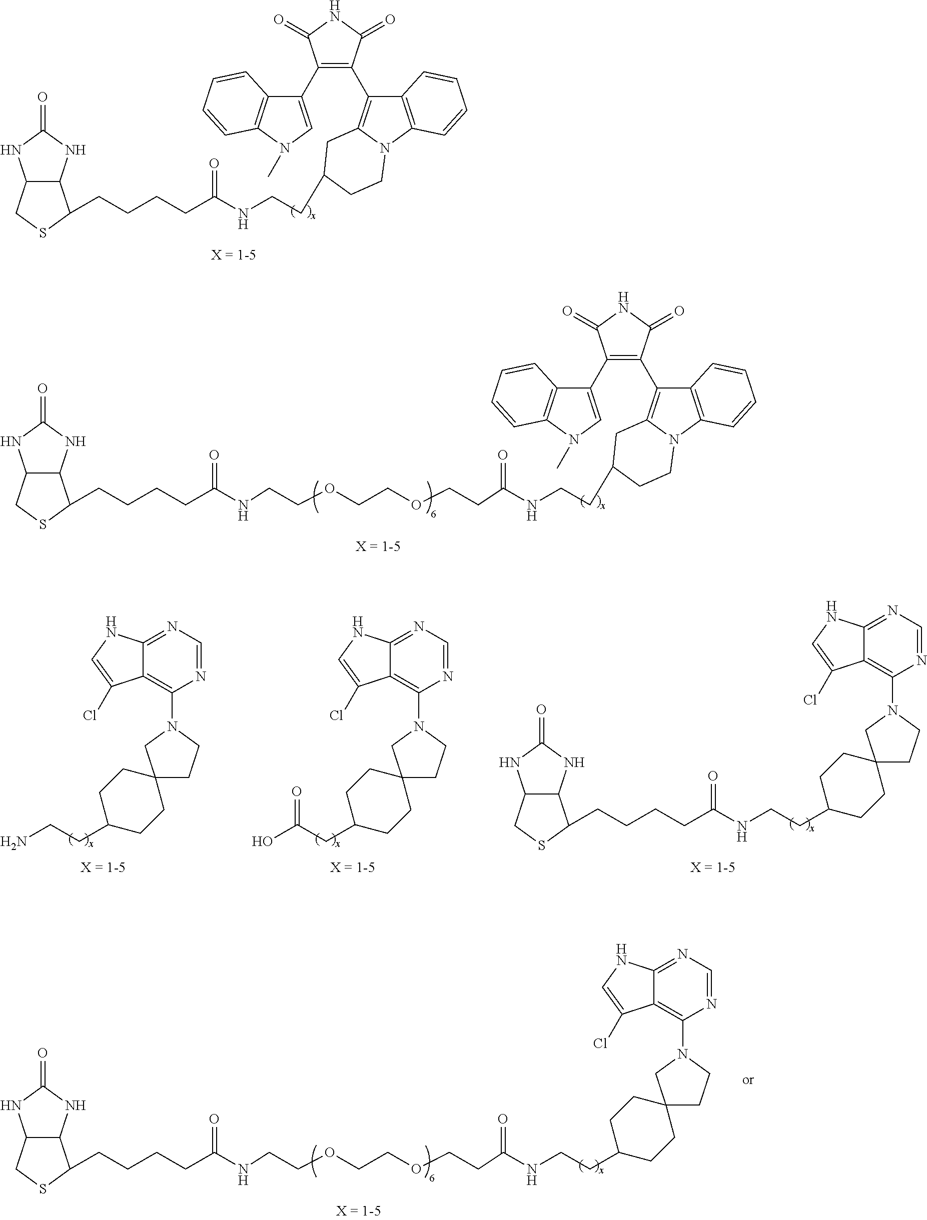

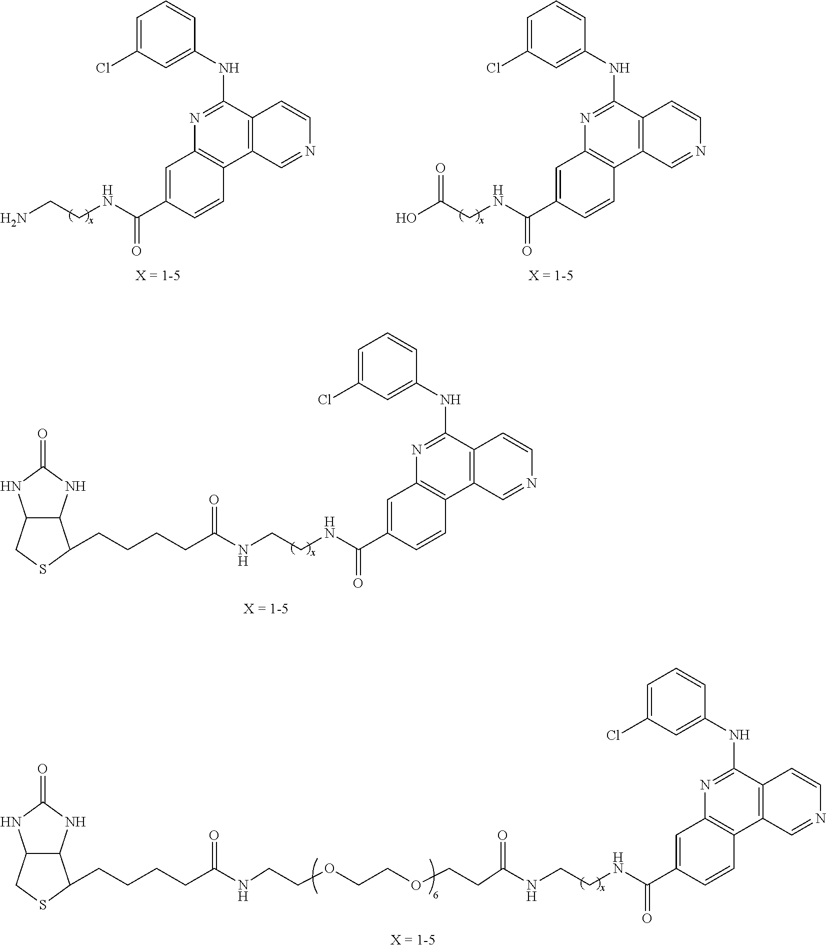

### 5.5. Coupling Methods

A wide variety of appropriate coupling methods may be used to attach the affinity ligands to a solid support. The coupling may be performed with covalent linkages such as amide linkages (e.g., amino NHS-ester), ester bonds, phosphoester bonds, or disulfide bonds. The coupling may also be performed using methods such as affinity tags, such as antigenic tags or other binding methods (e.g., antibody-protein A; biotin-streptavidin; FLAG-tag (Sigma-Aldrich, Hopp et al. 1988 Nat Biotech 6:1204-1210); glutathione S-transferase (GST)/glutathione; hemagluttanin (HA) (Wilson et al., 1984 Cell 37:767); intein fusion expression systems (New England Biolabs, USA) Chong et al. 1997 Gene 192(2), 271-281; maltose-binding protein (MBP)); poly His-(Ni or Co) (Gentz et al., 1989 PNAS USA 86:821-824); or thiol-gold. Fusion proteins containing GST-tags at the N-terminus of the protein are also described in U.S. Pat. No. 5,654,176 (Smith). Magnetic separation techniques may also be used such as Strepavidin-DynaBeads® (Life Technologies, USA). Alternatively, photo-cleavable linkers may be used, e.g., U.S. Pat. No. 7,595,198 (Olejnik & Rothchild). A wide variety of coupling methods, including polystyrene affinity peptides, are reviewed by Nakanishi et al. Nakanishi et al. 2008 Curr Proteomics 5 161-175, the contents of which and the other references of this section are hereby incorporated by reference in their entireties. Many other systems are known in the art and are suitable for use with the present invention.

### 5.6. Methods

The invention also provides a method for detecting low abundant kinases in a sample comprising: loading a sample on a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity; washing the multi-analyte column to remove any unbound proteins; eluting any kinases bound to the multi-analyte column with a denaturing agent; and detecting the eluted kinases.

The detection may be done by mass spectrometry. The method may be performed on a plurality of samples and at least one sample is labeled with a detectable label. The detectable label may be prepared by SILAC (stable isotope labeling with amino acids in cell culture). Alternatively, an isotope labeled spike is added to the sample.

In some embodiments, greater than 150 kinases are detected from 5 mg protein portion of the sample. In other embodiments, greater than 180 kinases are detected from the 5 mg protein portion of the sample. In other embodiments, 40 or more kinases are detected from a single sample and changes in phosphorylation states of the kinases are also measured.

In particular non-limiting embodiments, the invention provides a method of selecting a kinase activity modulator, the method comprising the steps of: contacting a cell, a tissue, or an organism with a compound; contacting a protein extract from the cell, the tissue, or the organism with a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity; eluting any kinases bound to the solid supports with a denaturing agent; measuring levels of a plurality of the kinases detected; comparing the levels measured previously to a standard level(s) to obtain a kinase profile; and using the kinase profile to select the kinase activity modulator.

The invention also includes a method for determining the prognosis of a cancer in a subject which method comprises: (a) measuring levels of a plurality of the kinases detected by the method above; and (b) comparing the levels measured in step (a) to a standard level, wherein modulation of the measured level of at least one kinase relative to the standard level indicates the prognosis of a cancer.

The invention also includes a method for improving effectiveness of treatment regimen for a kinase related disorder in a subject which method comprises: (a) measuring levels of a plurality of the kinases detected by the method above; (b) comparing the levels measured in step (a) to a standard level to obtain a kinase profile; and (c) using the kinase profile to determine a more effective treatment regimen.

The invention also includes a method for modifying a cancer therapy regimen which comprises: obtaining a sample from a patient; measuring levels of a plurality of the kinases detected by the method above; treating the patient with one or more kinase inhibitors; obtaining a second sample from the patient; measuring a plurality of kinases from the second sample; comparing the second sample levels to those measured previously; based on the comparison after treatment modifying the cancer therapy regimen.

The invention also includes a method for stratifying patients for a treatment regimen or a clinical trial which comprises (a) measuring levels of a plurality of the kinases detected by the method above; (b) comparing the levels measured in step (a) to a standard level to obtain a kinase profile; and (c) using the kinase profile to stratifying patients for the treatment regimen or the clinical trial. Such stratification could take place prior to treatment or in the course of treatment so as to determine whether or not kinase resistance is developing and to determine which additional kinase drugs would be complement the initial treatment. See e.g., McDermott et al. 2009 J Clin Oncol 27(33) 5650-5659.

The invention also includes a method for measuring a on-target or a off-target effect of a drug treatment which comprises (a) measuring levels of a plurality of the kinases detected by the method above; (b) comparing the levels measured in step (a) to a standard level to obtain a kinase profile; and (c) using the kinase profile to measuring the on-target or the off-target effect of the drug treatment.

A method of selecting a kinase activity modulator, the method comprising the steps of: contacting a cell, a tissue, or an organism with a compound; contacting a protein extract from the cell, the tissue, or the organism with a multi-analyte column comprising a first and a second layer wherein: the first layer comprises a first solid support having at least two different affinity ligands with specific kinase binding affinity; and the second layer comprises a second solid support having at least two different affinity ligands with non-specific kinase binding affinity; eluting any kinases bound to the solid supports with a denaturing agent; measuring levels of a plurality of the kinases detected; comparing the levels measured previously to a standard level(s) to obtain a kinase profile; and using the kinase profile to select the kinase activity modulator.

### 5.6.1. Method of Bead Design

One aspect of the invention would be drug discovery, specifically, preclinical small molecule development. Beginning with compounds of interest (lead compound, possibly found by combinatorial chemistry), one might follow these steps: (a) profile with purified target kinase for IC50; (b) profile kinome response to lead compounds using MIB/MS in relevant disease models (tumors, cell lines); (c) correlate MIB/MS kinome response profiles of lead compounds to structure activity relationship and IC50 for target kinase; and (d) define on-target/off-target activity of lead compounds based on the observed kinome reprogramming.

One could use information from the kinome reprogramming in response to select compounds that have a preferred toxicity profile. This could be used for a single drug, or a combination of one or more kinase inhibitors. Sessel and Fernandez describe modifications to drug scaffolds to alter hydrogen bonding, specificity, and on-target/off-target activities. Sessel and Fernandez, 2011 Curr Top Med Chem 11, 788-799. In a similar manner, the kinome reprogramming could be used to design improved drug combinations or test new drugs.

Many small molecule kinase inhibitors are ATP mimics and cardiomyocytes and other heart muscle cells consume large quantities of ATP. Thus, cardiotoxicity is major concern for this class of drugs. Force and Koloja, 2011 Nat Rev Drug Disc 10(2) 111-125; and Mellor et al., 2011, Tox Sci 120(1) 14-32. The MIB/MS techniques described herein could be used to study the cardiotoxicity profile of approved kinase inhibitors, kinase inhibitors currently in clinical trials, pre-clinical kinase inhibitors, or combinations of two, three, or more of these kinase inhibitors. More broadly, the MIB/MS techniques may be used to profile toxicity, safety, and efficacy profiles for kinase inhibitor combinations.

### 5.6.2. Design of MIBs for Patient Management

Another application of the invention would be to design custom multiplexed inhibitor beads (MIBs) to select patients or predict likely response to a single drug or a combination of drugs. (a) assess a MIB combination with patient samples, genetically engineered mouse models (GEMM), xenografts, or cell lines of interests. Examples of ligands on the MIBs are lapatinib, SB203580, bis-X, MEK inhibitor, AKT inhibitor, sorafenib, dasatinib, purvalanol B, PP58, VI16832, 2,4-diaminopyrimidine, pyrazole inhibitor. (b) Compare a MIB combination to exemplary criteria for MIB kinome profile (kinase family coverage): tyrosine kinase families (TK)=65%; tyrosine kinase-like families (TKL)=51%; homologues of yeast sterile 7, sterile 11, sterile 20 kinase families (STE)=55%; casein kinase 1 family (CK1)=67%; protein kinase A, G and C families (AGC)=42%; calcium/calmodulin-dependent protein kinase families (CAMK)=38%; CDK, MAPK, GSK, CLK families (CMGC)=66% (see Manning et al., 2002, Science 298, 1912-1918). (c) Define kinome signature for the disease type. (d) Define kinome reprogramming to inhibitor. (Criteria for determining kinase activated in response to inhibitor: >2-fold increase in MIB/MS binding). (e) Predict combination therapy based on MIB/MS kinome inhibitor response profile. (f) Preclinical test drug combinations using GEMMs, xenografts and cell lines. (g) Design custom MIBs to capture inhibitor-mediated kinome response for clinical window trials (see below). (h) Absolute quantitation of drug response using customized MIB/MS in patient tumor biopsies before and after drug treatment. (i) Predict drug response and design new therapeutic combinations for individual patients.

### 5.7. Clinical MIB Applications

### 5.7.1. Application of MIB/MS for Window Trials to Test Therapeutic Response to Drug

Using multi-analyte MIB columns a signature of therapeutic response and potential resistance to therapy is obtained from biopsy accessible tumors using so-called “window trials” in a clinical setting.

Study Synopsis:

A chemical proteomics approach using multi-analyte columns may be used to define the activity of a significant percentage (˜60-75%) of the expressed kinome in cells and tumors to predict therapeutic response. The technique involves the use of pan kinase inhibitors immobilized on beads to capture a large percentage of expressed kinases in cells and tumors. The activation state of the expressed kinome can be analyzed using mass spectrometry analysis of the captured kinases. This technique is used to study, and then rationally design a kinase inhibitor therapy with single agent or combination of agents for a specific cancer such as triple negative breast cancer (TNBC). The technique is applicable to any biopsy accessible cancer type. For example the biopsy may be from adrenal cortical cancer, anal cancer, aplastic anemia, bile duct cancer, bladder cancer, bone cancer, bone metastasis, brain/CNS tumors, breast cancer, cancer of unknown origin, Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal and hypopharyngeal cancer, leukemia—acute, lymphocytic (ALL), leukemia—acute myeloid (AML), leukemia—chronic lymphocytic (CLL), leukemia—chronic myeloid (CML), leukemia—chronic myelomonocytic (CMML), liver cancer, lung cancer—non-small cell (NSCLC), lung cancer—small cell, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, skin cancer—basal and squamous cell, skin cancer—melanoma, small intestine cancer, stomach cancer, testicular cancer, thymus cancer, thyroid cancer, uterine sarcoma, vaginal cancer, vulvar cancer, Waldenstrom macroglobulinemia, or Wilm's tumor

An example is a window trial for stage I-IV TNBC patients scheduled to undergo definitive surgery (either lumpectomy, mastectomy or surgical resection of oligometastatic disease). Enrolled patients will receive a defined dose of drug for 7-28 days (with final duration dependent on surgical schedule) prior to their surgery, with pre- and post-treatment tissue analyzed for kinome response and resistant signatures. Of note, the duration of study treatment is defined by the surgical schedule; there are no delays in standard treatment for the purposes of such studies.

Kinome Profiling Window Trials:

(1) Identify kinases that are differentially expressed in cancer patients tumors pre- and post-treatment with a drug. (2) Use differential kinase profiling using MIBs to predict rational single agent treatment and combinations of therapeutic drugs for optimal inhibition strategies to treat the cancer. (3) Perform whole kinome profiling pre- and post-drug treatment in patients and determine the baseline kinome pattern variability across a number of patients with the specific cancer.

Kinome Profiling of Clinical Samples:

(1) A core biopsy before drug treatment of a patient's tumor is collected by the surgeon. The sample is flash frozen to preserve the activation state of the kinome. (2) After the prescribed drug treatment (in some cases no drug may be given to the patient and the excised tumor is analyzed with no prior core biopsy sample), the tumor is surgically excised and preserved by flash freezing. Tumor kinases from the pre- and post-treatment are isolated using multi-analyte MIB columns and the kinome analyzed by mass spectrometry.

### 5.7.2. Human in Mouse Primary Tumor Models

Several investigators have recently reported the implantation of primary patient tumor cells in immune compromised mice to mimic the human cancer and/or design personalized patient treatments. In the literature these systems are variously called orthotopic tumor models, patient-derived xenographs (PDX), tumorgrafts, or xenografts. Examples include a breast cancer model reported by DeRose et al. and a NSCLC model reported by Dong et al. DeRose et al. 2011 Nat Med 17 1514-1520; Dong et al. 2010 Clin Cancer Res 16 1442-1451. Hidalgo et al. report another example using such human in mouse transplants to guide treatment for refractory advanced cancers such as NSCLC, CRC, or pancreatic cancer. Hidalgo et al. 2011 Mol Cancer Ther 10(8) 1311-1316. The kinome profiling methods described herein are well-suited to profile tumors using samples obtained from such models or to design, monitor, and refine specific treatment regimens involving a plurality of kinase inhibitors for a given cancer.

### 5.8. Biomarker Analysis Using Multi-Analyte MIB Capture of Kinases

For patient biopsy samples of limited protein content peptides labeled with heavy amino acids (heavy peptides) will be used for quantitation of kinases that are at very low concentrations in the complex kinome mixture. Heavy peptides of different mass for 100s of different kinases can be added to a single kinase preparation from a patient biopsy. (2) Heavy peptides generally consist of up to 15 amino acids and labeled with 15N, 13C or 2H-labeled amino acids. The peptides represent the natural proteolytic fragments of kinases such PDGFRβ, VEGFR2, DDR1, Src, AKT, RSK1, etc. Heavy peptides can be made for each of the kinases within the kinome representing 518 different kinases. In addition, phosphorylated heavy peptides can be synthesized that represent, for example, the activated state of the kinase, where the non-phosphorylated heavy peptide would represent the inactive form of the kinase. (Gerber et al., 2003 Proc. Natl. Acad. Sci. U.S.A. 100: 6940-6945). (3) Heavy peptides representing specific kinases in the non-phosphorylated (inactive) and phosphorylated (active) states of the kinase will be added to proteolytic digests of kinases isolated from patient biopsies using multi-analyte MIB columns. The samples will be analyzed by mass spectrometry and for absolute quantitation of the “active versus inactive state of the kinase” (kinase activation ratio). (4) The kinase activation ratio is used to define the activity state of the kinase in a tumor biopsy and if specific drug treatments activate or inhibit the kinase during treatment. Examples would be heavy peptides for the phosphorylated and nonphosphorylated activation loops of different receptor tyrosine kinases, cytoplasmic tyrosine kinases or serine/threonine kinases captured on the multi-analyte MIB column.

### 5.9. Customizing Multi-Analyte MIB Columns for Cancer-Specific Patient Kinase Profiling

Profiling a significant number of patient tumors using multi-analyte MIB/MS analysis combined with RNA-seq and gene array data from many different cancer types will provide a cancer kinome roadmap. RNA-seq and gene array data will define expression of kinases in different tumors, whereas MIB/MS provides the critical activation state of the kinome in different cancers. This data will be used to customize the composition of the multi-analyte MIB columns used for patient biopsy analysis. For tumors expressing EGFR tyrosine kinases (ERBB1-4) lapatinib coupled beads would be included in the multi-analyte column. For tumor expressing tyrosine kinases including PDGFRα and β, DDR1 and 2, VEGFR², and Ron a sorafenib coupled bead would be included in the multi-analyte column. Customizing multi-analyte columns is based on the kinase expression profile of the tumor and the inhibitor-bead binding profile previously determined in FIG. 8B. The customized multi-analyte columns will consist of at least three different inhibitor beads and will enhance training on specific kinase biomarkers in patient samples using heavy peptide quantitation methods as described above.

### 5.10. Mass Spectrometry Methods

In one preferred embodiment, the proteins bound to the multi-plexed beads are analyzed by mass spectroscopy (MS). A wide variety of mass spectroscopy techniques are known in the art, e.g., Mann et al., 2001, Ann Rev Biochem 70, 437-473, Wissing et al., 2007, Mol Cell Proteo 6 537-547. For example, tandem MS, Gerber et al., 2003, Proc. Natl. Acad. Sci. 100: 6940-6945, PCT Patent Pub. No. WO 2006/134056 (Drewes et al.); acyl phosphate irreversible probes based on ADP or ATP, Patricelli et al., 2011 Chem Biol 18 699-710, Patricelli et al., 2007 Biochem 46 350-358; AQUA Peptides developed by Gygi and colleagues [Stemmann et al., 2001 Cell 2001 107: 715-726 cICAT (cleavable isotopic-coded affinity tags) Wu et al., 2006, J Proteome Res 5, 651-658; iTRAQ (isobaric tags for absolute and relative quantification) Bantscheff et al., 2007, Nat Biotech 25(9) 1035-1044, Ross et al., 2004, Mol Cell Proteo 3 1154-1169; MRM MS (multiple reaction monitoring mass spectrometry) Hardt et al., 2008 Thermo Scientific Application note: 451, Kuhn et al., 2004, Proteomics 4 1175-1186; SILAC (stable isotope labeling with amino acids in cell culture), Pub. Appn. No. US 2010/0279891 (Daub et al.), Daub et al., 2008 Mol Cell 31 438-448, Ong et al., 2002 Mol Cell Proteo 1 376-386; super SILAC, a spike-in mix for SILAC, Geiger et al., 2010 Nat Meth 7(5) 383-387, Geiger et al., 2011 Nat Prot 6(2) 147-157; titanium dioxide enrichment of phosphopeptides, Thingholm et al., 2006 Nat Prot 1(4) 1929-1935; the contents of which are hereby incorporated by reference in their entireties.

A variety of methods have been reported for analysis of peptides and proteins including iProphet and PeptideProfit used herein. iProphet, Shteynberg et al., 2011 Mol Cell Proteomics 10 M111.007690 1-15; PeptideProphet, Keller et al., 2002 Anal. Chem. 74, 5383-5392. Other methods for mass spectral analysis include Inspect, Mascot, MyriMatch, OMSSA, SEQUEST, X! Tandem. See Inspect, Tanner et al., 2005 Anal Chem 77, 4626-4639; Mascot, Perkins et al., 1999 Electrophoresis 20, 3551-3567; MyriMatch, Tabb et al., 2007 J Proteome Res 6 654-661; OMSSA, Geer et al., 2004 J Proteome Res 3 958-964; SEQUEST, Eng et al., 1994 J Am Soc Mass Spectrom 17 2310-2316; X! Tandem, Craig et al., 2003 Rapid Comm Mass Spectrom 17 2130-2316. The contents of which are hereby incorporated by reference in their entireties.

### 5.11. Combination Methods

The multi-analyte columns of the invention are well-suited for use in combination with other methods of cancer diagnosis, prognosis, staging, particularly those for solid tumors. Methods such as antibody staining, comparative genomic hybridization (CGH), cytogenetics, fluorescent in situ hybridization (FISH), genotyping (SNP analysis), hematoxylin and eosin (H&E) staining, mRNA expression profiling, methylation profiling are known and kits or services are commercially available. Examples of such techniques may be found in references such as Igbokwe et al., 2011, Arch Pathol Lab Med 135 67-82 and Monzon and Koen, 2010, Arch Pathol Lab Med 134 216-224, the contents of which are hereby incorporated by reference in their entireties.

### 5.12. Methods to Identify Compounds

A variety of methods may be used to identify compounds that modulate a kinase related disorder and prevent or treat a kinase related disorder progression. Typically, an assay that provides a readily measured parameter is adapted to be performed in the wells of multi-well plates in order to facilitate the screening of members of a library of test compounds as described herein. Thus, in one embodiment, an appropriate number of cells can be plated into each well of a multi-well plate, and the effect of a test compound on a kinome profile associated with a kinase related disorder can be determined. The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to modulate a kinome profile associated with a kinase related disorder. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries. See, e.g., U.S. Pat. No. 5,010,175 (Rutter and Santi), Furka 1991 Int. J. Pept. Prot. Res., 37:487-493; and Houghton et al., 1991 Nature 354:84-88. Other chemistries for generating chemical diversity libraries can also be used. Such chemistries include, but are not limited to: U.S. Pat. No. 6,075,121 (Bartlett et al.) peptoids; U.S. Pat. No. 6,060,596 (Lerner et al.) encoded peptides; U.S. Pat. No. 5,858,670 (Lam et al.) random bio-oligomers; U.S. Pat. No. 5,288,514 (Ellman) benzodiazepines; U.S. Pat. No. 5,539,083 (Cook et al.) peptide nucleic acid libraries; U.S. Pat. No. 5,593,853 (Chen and Radmer) carbohydrate libraries; U.S. Pat. No. 5,569,588 (Ashby and Rine) isoprenoids; U.S. Pat. No. 5,549,974 (Holmes) thiazolidinones and metathiazanones; U.S. Pat. No. 5,525,735 (Takarada et al.) and U.S. Pat. No. 5,519,134 (Acevado and Hebert) pyrrolidines; U.S. Pat. No. 5,506,337 (Summerton and Weller) morpholino compounds; U.S. Pat. No. 5,288,514 (Ellman) benzodiazepines; diversomers such as hydantoins, benzodiazepines and dipeptides (Hobbs et al., 1993, Proc. Nat. Acad. Sci. USA, 90, 6909-6913), vinylogous polypeptides (Hagihara et al., 1992, J. Amer. Chem. Soc., 114, 6568), nonpeptidal peptidomimetics with glucose scaffolding (Hirschmann et al., 1992, J. Amer. Chem. Soc., 114, 9217-9218), analogous organic syntheses of small compound libraries (Chen et al., 1994, J. Amer. Chem. Soc., 116:2661 (1994)), oligocarbamates (Cho et al., 1993, Science, 261, 1303 (1993)), and/or peptidyl phosphonates (Campbell et al., 1994, J. Org. Chem., 59:658), nucleic acid libraries (see Ausubel, Berger and Sambrook, all supra); antibody libraries (see, e.g., Vaughn et al., 1996, Nat. Biotech., 14(3):309-314, carbohydrate libraries, e.g., Liang et al., 1996, Science, 274:1520-1522, small organic molecule libraries (see, e.g., benzodiazepines, Baum, 1993, C&EN, January 18, page 33. Devices for the preparation of combinatorial libraries are commercially available (see, e.g., 357 MPS, 390 MPS, Advanced Chem Tech, Louisville Ky., Symphony, Rainin, Woburn, Mass., 433 A Applied Biosystems, Foster City, Calif., 9050 Plus, Millipore, Bedford, Mass.). In addition, numerous combinatorial libraries are themselves commercially available (see, e.g., ComGenex (Princeton, N.J.), Asinex (Moscow, RU), Tripos, Inc. (St. Louis, Mo.), ChemStar, Ltd., (Moscow, RU), 3D Pharmaceuticals (Exton, Pa.), Martek Biosciences (Columbia, Md.), etc.).

Methylation modifiers are known and have been the basis for several approved drugs. Major classes of enzymes are DNA methyl transferases (DNMTs), histone deacetylases (HDACs), histone methyl transferases (HMTs), and histone acetylases (HATs). DNMT inhibitors azacitidine (Vidaza®) and decitabine have been approved for myelodysplastic syndromes (for a review see Musolino et al., 2010 Eur. J. Haematol. 84, 463-473; Issa, 2010 Hematol. Oncol. Clin. North Am. 24(2), 317-330; Howell et al., 2009 Cancer Control, 16(3) 200-218; which are hereby incorporated by reference in their entirety). HDAC inhibitor, vorinostat (Zolinza®, SAHA) has been approved by FDA for treating cutaneous T-cell lymphoma (CTCL) for patients with progressive, persistent, or recurrent disease. Marks and Breslow, 2007 Nat. Biotech. 25(1), 84-90. Specific examples of compound libraries include: DNA methyl transferase (DNMT) inhibitor libraries available from Chem Div (San Diego, Calif.); cyclic peptides (Nauman et al., 2008 ChemBioChem 9, 194-197); natural product DNMT libraries (Medina-Franco et al., 2010 Mol. Divers., 15(2):293-304); HDAC inhibitors from a cyclic α3β-tetrapeptide library (Olsen and Ghadiri, 2009 J. Med. Chem. 52(23), 7836-7846); or HDAC inhibitors from chlamydocin (Nishino et al., 2006 Amer. Peptide Symp. 9(7), 393-394).

### 5.13. Kits

Kits are also provided comprising: multi-analyte column with a first and a second layer wherein: the first layer comprises a first solid support having at least two different affinity ligands with specific kinase binding affinity; and the second layer comprises a second solid support having at least two different affinity ligands with non-specific kinase binding affinity; and instructions for use in measuring level of a plurality of kinases in a subject who has cancer or been previously treated with a chemotherapy regimen.

A kit may optionally further comprise a container with a predetermined amount of a purified kinase, a peptide from a kinase or a phosphopeptide from a kinase, for use as a standard or control useful in quantifying the amount of kinases in the sample. It may include isotopically labeled materials such as C13, N15, or O18. Each kit may also include printed instructions and/or a printed label describing the practicing of the invention in accordance with one or more of the embodiments described herein. Kit containers may optionally be sterile containers. The kits may also be configured for research use only applications whether on clinical samples, research use samples, cell lines and/or primary cells. The kits, beads, and/or columns may also be configured for uses such as drug discovery (e.g., method of compound identification in Section 5.13 above); compound validation; optimizing a therapeutic window; investigating kinome toxicity (e.g., cardiotoxicity) for in vitro, in vivo systems, animal models (cell line xenographs, primary human cell xenographs in animals). The kits may also be configured for understanding a mechanism or action or basic research into the kinome in any of these systems. One of ordinary skill would readily understand a myriad of uses of the tools and methods described herein to study and improve kinome associated disorders human, animal, plant diseases, particularly cancers. The kinome has great importance learning, immunological disorders and developmental biology. With regard to plant kinomes, see exemplary studies of the rice kinome and the Arabidopsis kinome. Dardick et al. 2007 Plant Physiology 143(2) 579-586; Ritsema et al. 2007 Plant Methods 3:3 doi:10.1186/1746-4811-3-3.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

The following Examples further illustrate the invention and are not intended to limit the scope of the invention.

### 6. EXAMPLES

### 6.1. Introduction

Kinase inhibitors have limited success in cancer treatment because tumors circumvent their action. The kinome activity in response to MEK inhibition was assessed in triple negative breast cancer (TNBC) cells and genetically engineered mice (GEMMs). MEK inhibition caused acute loss of ERK activity, resulting in rapid c-Myc degradation that induced expression and activation of several receptor tyrosine kinases (RTKs). RNAi knockdown of ERK or c-Myc mimicked RTK induction caused by MEK inhibitors, whereas prevention of c-Myc degradation by proteasome inhibition blocked kinome reprogramming. MEK inhibitor induced RTK stimulation overcame MEK2 but not MEK1 inhibition, reactivating ERK and producing drug resistance. The C3Tag GEMM for TNBC similarly induced RTKs in response to MEK inhibition. The inhibitor-induced RTK profile suggested a kinase inhibitor combination therapy that produced GEMM tumor apoptosis and regression where single agents were ineffective. This approach defines mechanisms of drug resistance and allows rational design of combination therapies for cancer.

A chemical proteomics approach was developed to define the activity and drug responsiveness of a significant percentage of the expressed kinome in cells and tumors. The application of this technique allowed rational design of an effective kinase inhibitor combination therapy for triple negative breast cancer (TNBC), which currently lacks successful targeted treatments. Analysis of patient TNBC showed activated RAF-MEK1/2-ERK1/2 (hereafter referred to as MEK and ERK) signaling, supporting MEK as a target in TNBC. However, following MEK inhibition in TNBC cell lines and GEMM tumors, the kinome was rapidly reprogrammed by the induced expression and activity of Tyr and Ser/Thr kinases that bypassed the original MEK-ERK inhibition. Using this global technique, previously undiscovered Tyr and Ser/Thr kinase activation was observed in response to MEK inhibitors by a robust mechanism of kinome reprogramming that involved differential sensitivity of MEK1 and MEK2 and loss of the transcription factor c-Myc. From the MEK inhibitor kinome response signature, a novel small molecule combination therapy for TNBC was predicted and tested. The combination synergistically inhibited TNBC cell line proliferation and caused apoptosis and tumor regression in the C3Tag GEMM of basal-like/claudin-low TNBC.

### 6.2. Results Kinome Profiling of TNBC

TNBC clinical trials of single kinase inhibitors have largely failed, consistent with drug-induced activation of alternative survival signaling pathways. FIG. 1A outlines the strategy to interrogate kinome dynamics with the goal of defining endpoints leading to rational design of combination therapies. RNA-seq defined the transcript-level expressed kinome and affinity capture of endogenous kinases followed by quantitative mass spectrometry measured kinome activity profiles in tumors and cells. The proteomic assessment was used to define the kinome response to targeted inhibition of kinases. RNAi tested growth and survival functions of the kinases activated in response to inhibitors, and the cumulative results were used to rationally predict kinase inhibitor combinations to test in models of TNBC.

RNA-seq defined the kinome transcript expression profile of a patient's claudin-low breast tumor and two claudin-low TNBC lines, SUM159 and MDA-MB-231. Greater than 400 of the 518 human protein kinase transcripts are expressed in the claudin-low human TNBC tumor and cell lines (FIG. 1B). Approximately 10% of the kinases expressed in the claudin-low patient tumor were unique compared to the claudin-low cell lines, undoubtedly due to the tumor's complex cellular composition. The majority of expressed kinases are common between tumor and claudin-low cell lines, suggesting that interrogating the cellular kinome response to inhibitors will be relevant to patient tumors.

Profiling kinase activity in tumors and cell lines was carried out using Multiplexed Inhibitor Beads (MIBs), which consist of mixtures of Sepharose beads with covalently immobilized, linker adapted, kinase inhibitors of moderate selectivity for different kinases (specific kinase binding affinity) (bisindolylmaleimide-X, SB203580, dasatinib and lapatinib) and relatively broad pan-kinase inhibitors (non-specific kinase binding affinity) (VI16832, purvalanol B, and PP58) (FIG. 8A). Daub et al., 2008. Kinase capture is reproducible and is a function of kinase expression, the affinity of kinases for the different immobilized inhibitors, and the activation state of the kinase. Bantscheff et al., 2007. Acute changes in activation-dependent binding were demonstrated by the increased binding of MAPK pathway kinases in EGF-stimulated cells and the increased retention of Tyr kinases from cells treated with the Tyr phosphatase inhibitor pervanadate (FIGS. 8B and 8C). The data showed that MIBs capture the majority of the expressed kinome estimated by RNA-seq and detect altered kinome activity profiles in response to stimulus or kinase inhibitors used to treat cancer.

Using MIBs and mass spectrometry, cumulatively more than 320 expressed kinases from cell lines and tumors (Table 3) sequence identified. MIB/MS profiling of an invasive ductal carcinoma breast tumor and the two claudin-low cell lines identified approximately 50-60% of the kinome expressed at the transcript level (FIG. 1C-1E). Kinases from all major kinome subfamilies were captured with a large percentage representing the untargeted kinome (Fedorov et al., 2010). Additionally, iTRAQ labeling of peptides derived from MIB purified kinases was used to quantitatively profile kinases in the patient invasive ductal carcinoma compared to adjacent uninvolved mammary tissue (FIG. 1F). Of the kinases detected, there was a general increase in MIB binding of tumor kinases, suggesting escalated kinome activity in the tumor compared to uninvolved mammary tissue. For example, the RAF-MEK-ERK pathway is increased in MIB binding in the tumor relative to control tissue, consistent with ERK activity being a driver for TNBC proliferation Immunoblots confirmed the activation of RAF-MEK-ERK signaling in the patient invasive ductal carcinoma (FIG. 1G). RTK arrays were used to confirm Tyr phosphorylated RTKs in the patient invasive ductal carcinoma and a claudin-low breast tumor, which showed phosphorylation of EGFR, HER2, PDGFRβ, CSF1R, RON and EPHB2 (FIG. 1H). Although the data pointed to the potential importance of Tyr phosphorylated EGFR and PDGFRβ in the patient tumors, clinical trials targeting these RTKs have largely failed. Bianchi et al., 2009 Anticancer Drugs 20 616-624; Finn et al., 2009 J Clin Oncol 27 3908-3915. The failure of single agent RTK inhibitors in TNBC is consistent with drug-induced activation of multiple kinases or compensatory tumor kinome responses. Since many expressed RTKs drive ERK activation, claudin-low breast cancer cells were profiled after MEK inhibition (e.g. AZD6244 currently in clinical trials), to determine if dynamic kinome reprogramming occurs. One goal was to define kinome alterations that would suggest a more effective, rationally designed combination therapy.

### 6.3. Reprogramming the Kinome in Response to MEK Inhibition

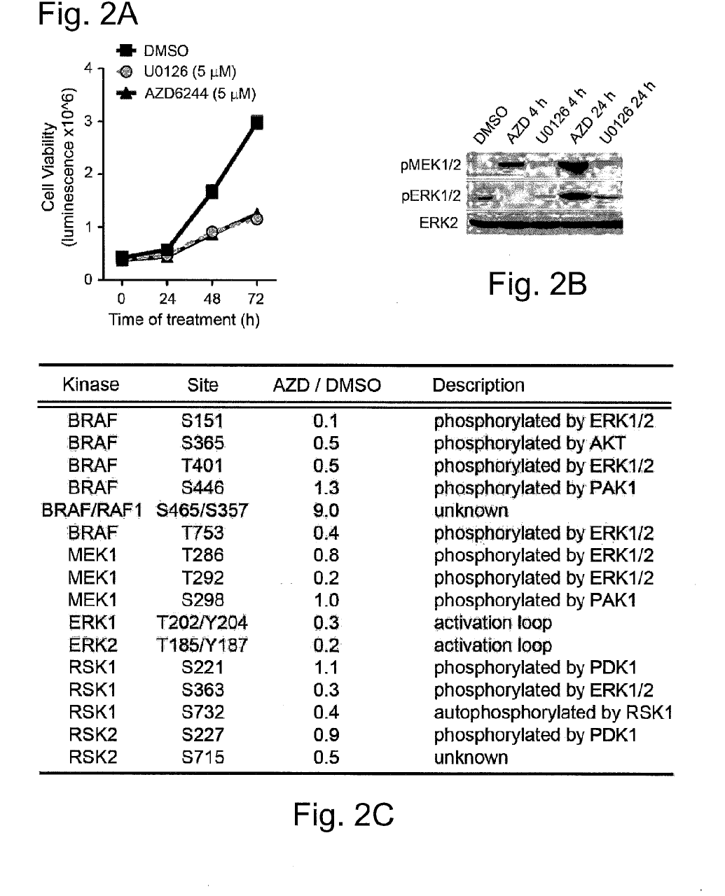

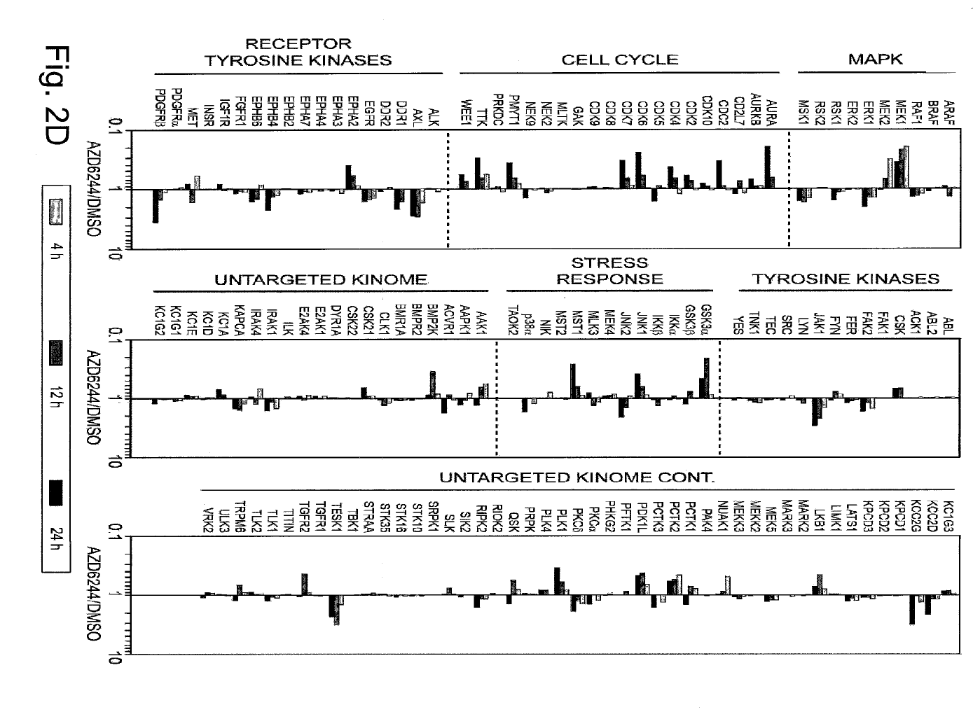

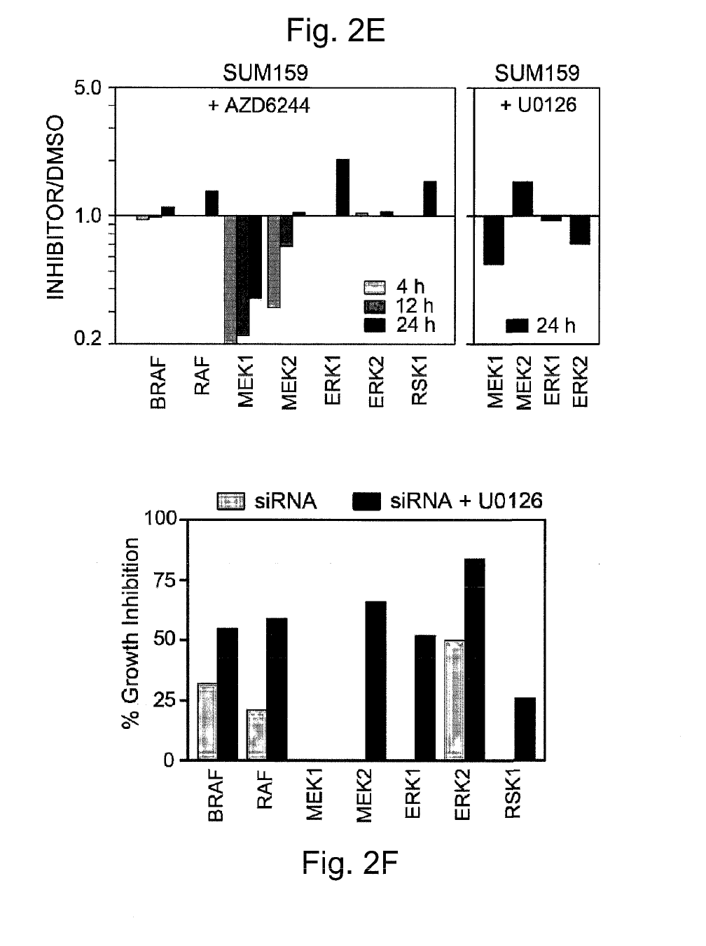

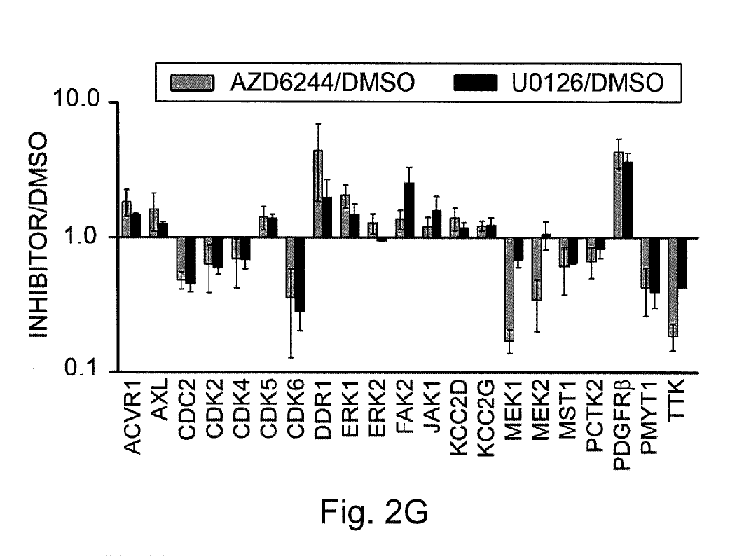

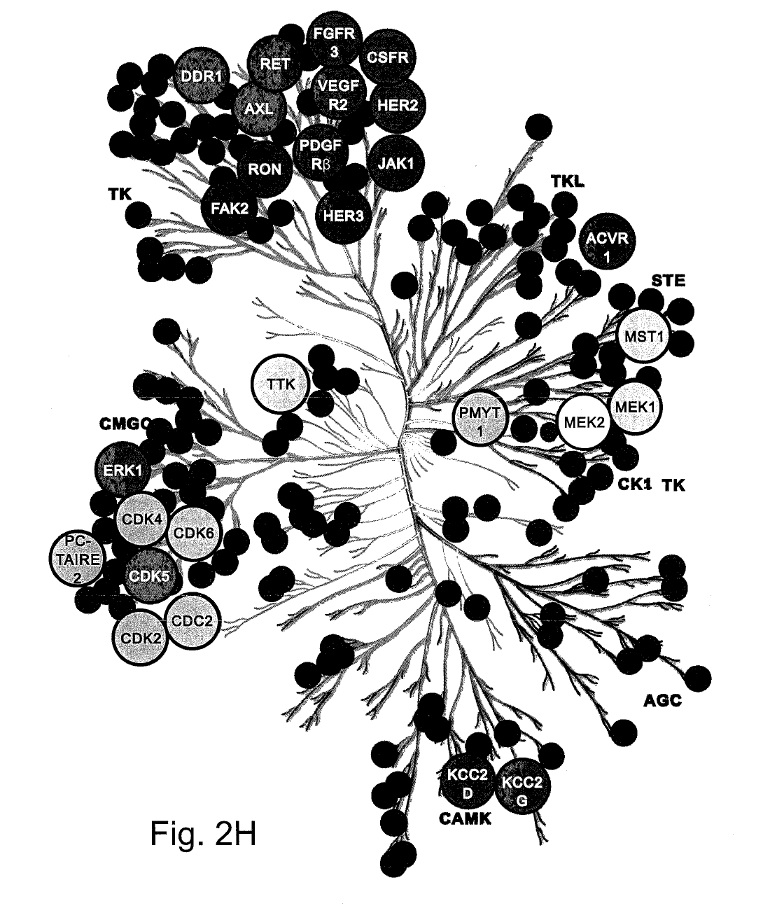

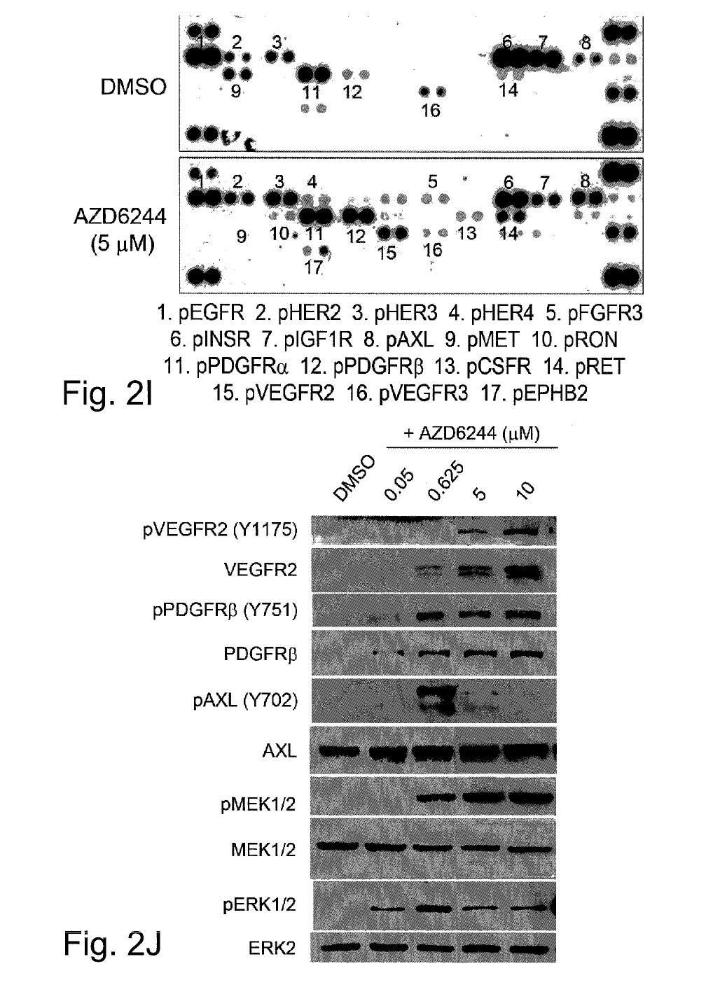

MEK inhibitors AZD6244 or U0126 inhibited growth of SUM159 (FIG. 2A) and MDA-MB-231 cells (FIG. 9A). Four hours after exposure to MEK inhibitor, ERK remained inhibited while MEK phosphorylation, as defined by a phosphoantibody that recognizes both MEK1 and MEK2, was enhanced (FIG. 2B) Inhibitor treatment for 24 h resulted in further activation of both MEK and ERK, demonstrating both lines overcame the initial MEK inhibition (FIGS. 2B and 9B). Phosphoproteomic analysis revealed loss of ERK mediated feedback regulation of both BRAF and MEK1 (FIG. 2C). Reduction of the ERK phosphorylation sites on BRAF and MEK1 (these are negative feedback regulatory sites) indicate escape from the suppressive feedback effects on the ERK pathway. Ritt et al. 2010 Mol Cell Biol 30, 806-819. Analysis of MIB isolated protein kinases identified 52 peptides with decreased and 59 peptides with increased phosphorylation, while the phosphorylation status of 365 phosphopeptides was unchanged after MEK inhibition (FIG. 9C). The majority of these phosphorylation sites were Ser, Thr and Pro-directed Ser/Thr sites, but P-Tyr changes were also included, suggesting a broad change in kinome activity in response to AZD6244.

We next used MIB/MS to profile the SUM159 kinome response after 4, 12 and 24 h exposure to AZD6244 (FIG. 2D). MEK inhibition resulted in a profile of time-dependent MIB binding changes for more than 140 kinases. Changes in MIB binding were seen for cell cycle regulatory kinases, MAPK pathway kinases, RTKs, cytosolic Tyr kinases and many Ser/Thr kinases. FIG. 2E highlights the MIB binding dynamics for MAPK component kinases during the time course of MEK inhibitor response of SUM159 cells. At 4 h of AZD6244 treatment both MEK1 and MEK2 are inhibited, as measured by loss of MIB binding. However, while MEK1 binding remains largely inhibited, MEK2 binding to MIBs increases at 12 h of treatment and by 24 h was similar to control cells (AZD6244/DMSO ratio=1), indicating a return of MEK2 activity. In parallel to restored MEK2 binding to MIBs, RAF1 and ERK1 binding to MIBs increases over the time course of AZD6244 treatment, correlating with activation of these kinases. RNAi was used for each kinase in the MAPK pathway to determine if knockdown of expression had a differential growth affect in response to MEK inhibition (FIG. 2F). RNAi knockdown shows that loss of MEK2 and ERK1 inhibited SUM159 cell growth in the presence of MEK inhibitor, whereas MEK1 knockdown did not produce enhanced growth inhibition. Consistent with the MIB/MS analysis, MEK2 and ERK1 are the MEK/ERK kinases that escape from inhibition by AZD6244, indicating they are critical for SUM159 cell growth in the presence of AZD6244.

FIGS. 2G and 2H show a 21 kinase signature defining a reprogrammed kinome in response to MEK inhibitor. The 21 kinase signature was defined using the method outlined in Section 6.18 for 3 biological replicate experiments. Statistical changes in kinase MIB binding across all 3 replicates following drug treatment qualifies as a drug responsive kinase. The signature will depend on the cancer type assayed, e.g., glioblastoma, head and neck cancer, hepatocellular carcinoma (HCC), hormone refractory metastatic prostate cancer, melanoma, metastatic colon cancer, non-small cell lung cancer (NSCLC), or pancreatic cancer. The fold differences are the average from three independent experiments treating SUM159 cells with AZD6244 or U0126. This signature shows the loss of cyclin dependent kinases, consistent with growth inhibition and increasing ERK binding to MIBs indicating MEK inhibition was being overcome. RTKs including AXL, DDR1 and PDGFRβ, cytosolic Tyr kinases FAK2 and JAK1, and the activin receptor Ser kinase all showed increased MIB binding. While MDA-MB-231 cells have a somewhat less robust kinome response to AZD6244, they displayed a significant kinome reprogramming including a strong increase in PDGFRβ binding to MIBs (FIG. 9D).

RTK arrays confirm the increased Tyr phosphorylation of multiple RTKs, including PDGFRβ in response to MEK inhibition (FIGS. 2I and 9E). In SUM159 cells VEGFR2, AXL and RET also have significantly increased Tyr phosphorylation in response to AZD6244. The AZD6244 response of SUM159 cells is dose-dependent (FIG. 2J), as PDGFRβ and VEGFR2 show increased Tyr phosphorylation and protein expression with increasing concentration of AZD6244. These results demonstrate that a significant number of kinases were induced in response to MEK inhibition. Relevant to the changes in the kinome to MEK inhibition, FIG. 9F lists the 40 highest expressed kinase transcripts of a patient claudin-low tumor. Of these 40 kinases, 14 (24%) were dynamically regulated in binding to MIBs in SUM159 and/or MDA-MB-231 cells in response to AZD6244, suggesting patient tumors could have a similar dynamic kinome reprogramming in response to targeted kinase inhibition.

### 6.4. MEK Inhibition Deregulates Transcription, Expression and Activation of RTKs

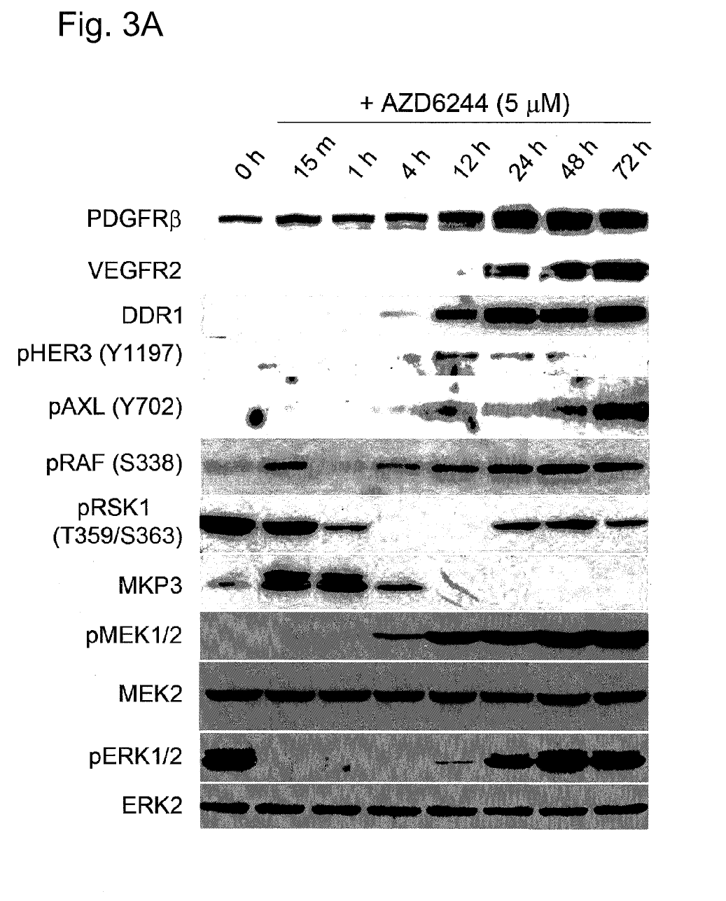

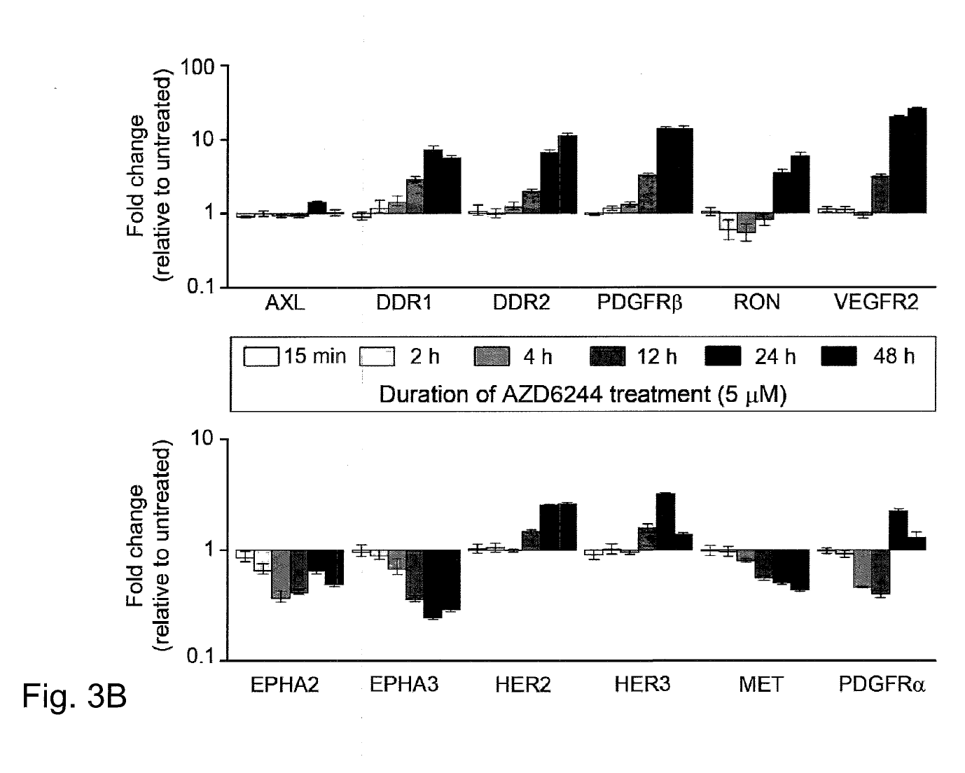

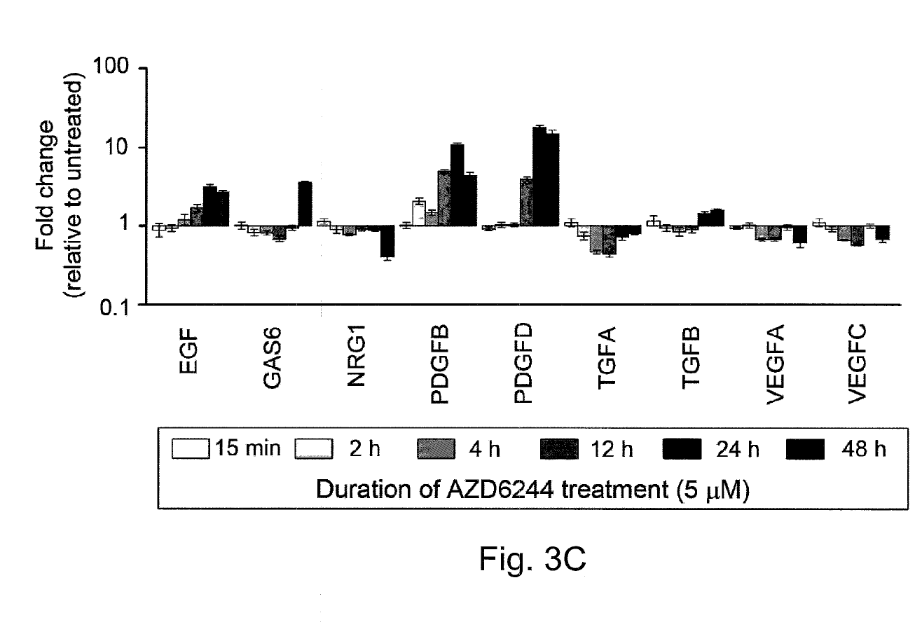

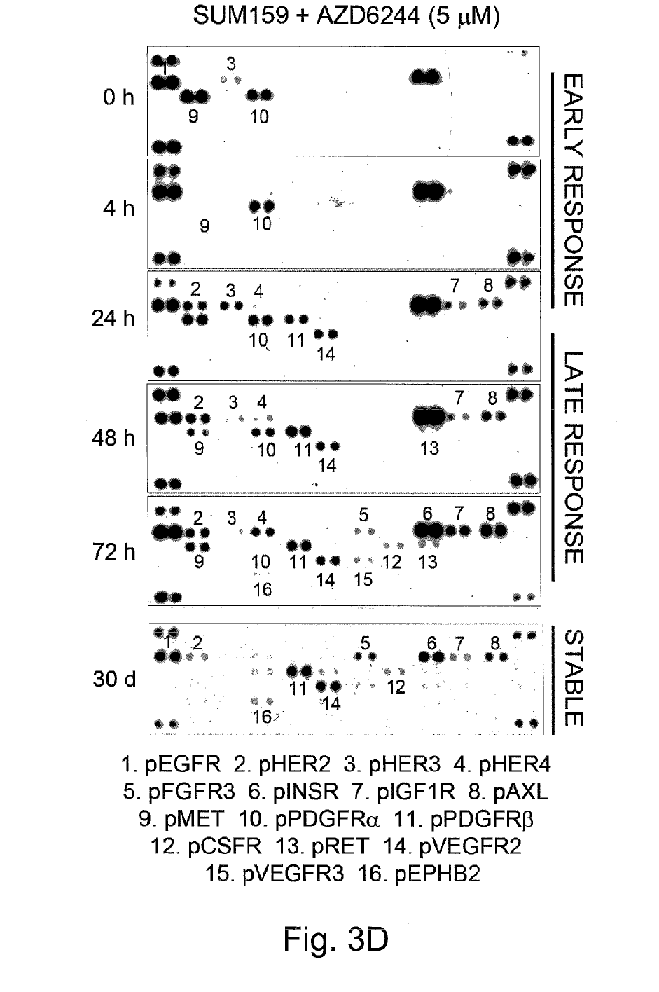

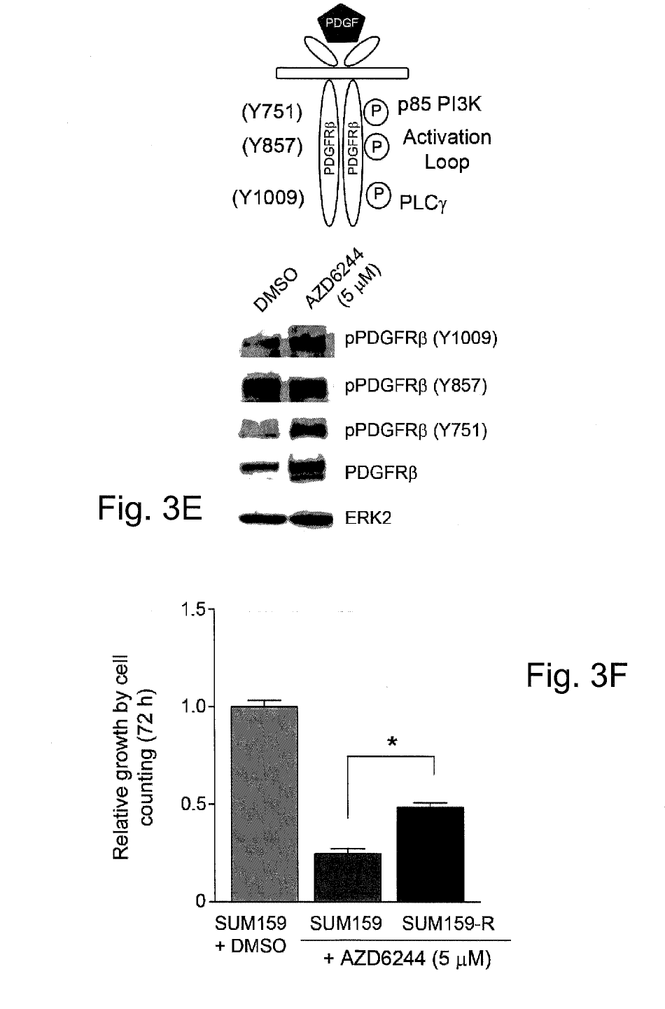

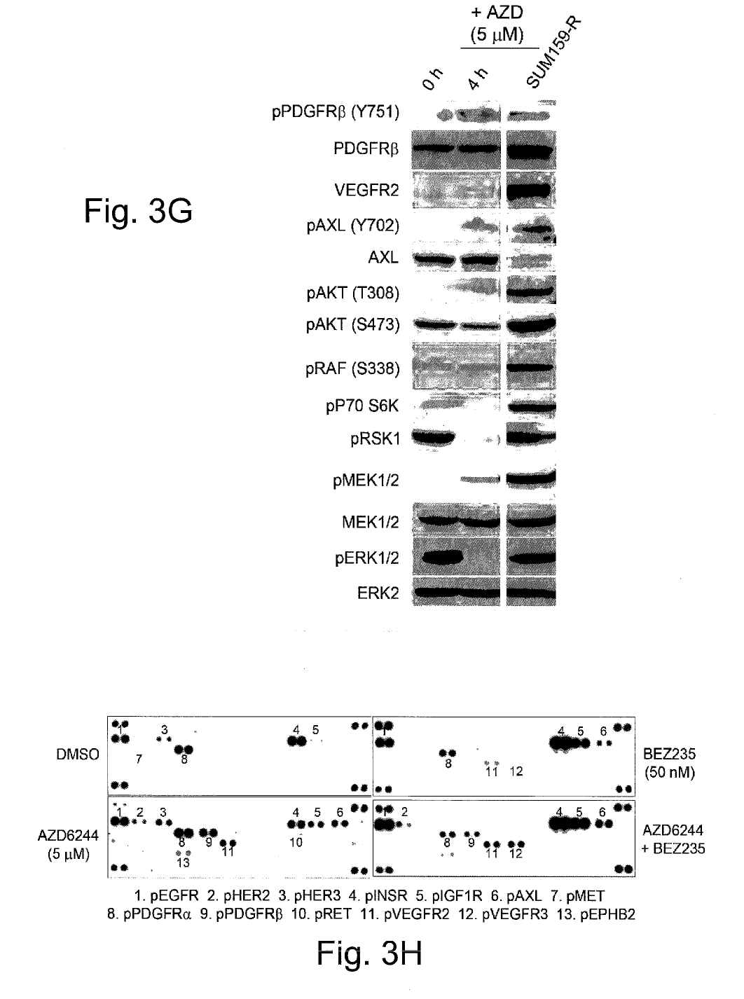

FIGS. 3A and 10A defines the early (15 min-4 h) and late (12-72 h) reprogramming response to AZD6244 in SUM159 and MDA-MB-231 cells. MEK and ERK were rapidly inhibited, allowing accumulation of MKP3, the MAPK phosphatase that inactivates ERK. Sandrine Marchetti et al. 2005 Mol Cell Biol 25 854-864. Increased MKP3 expression combines with the MEK inhibitor to strongly suppress ERK activity, but MKP3 protein is lost as MAPK pathway activity returns. Over time, VEGFR2, PDGFRβ and DDR1 expression was increased with AZD6244 treatment, as was the phosphorylation of HER3 and AXL. qRT-PCR analysis of SUM159 (FIG. 3B) and MDA-MB-231 (FIG. 10B) cells treated with AZD6244 demonstrated altered RNA levels for several of these RTKs. By 12 h, SUM159 cell transcripts for DDR1, DDR2, PDGFRβ, RON, VEGFR2, HER2 and HER3 were increased. MDA-MB-231 cells showed increased DDR1, DDR2, PDGFRβ, EPHA3, HER2 and HER3 transcripts. Analysis of cytokine RNA expression showed EGF, Gash, PDGFB and PDGFD induction, indicating the establishment of autocrine/paracrine loops for RTK activation (FIG. 3C and 10C). RTK phospho-antibody arrays further showed a time dependent increase in Tyr phosphorylation of PDGFRβ, VEGFR2, HER2 and HER3 (DDR1 and DDR2 are not on the array) (FIG. 3D). PDGFRβ, whose RNA and protein expression was induced in response to AZD6244, was phosphorylated at Tyr 751, 857 and 1009; sites required for receptor kinase activation and recruitment of PI3K and PLCγ (FIG. 3E).

After 30 days of continuous exposure to AZD6244, SUM159 cells have become significantly resistant to MEK inhibitor-induced growth arrest (FIG. 3F). Expression of cyclins A2 and B1 have recovered, consistent with increased proliferation (FIG. 10D). The AZD6244-resistant cells (SUM159-R) continue to have a reprogrammed kinome where PDGFRβ and VEGFR2 exhibited both increased expression and Tyr phosphorylation, and AXL showed increased Tyr phosphorylation (FIGS. 3G and 3H). Activation of these RTKs is accompanied by increases in phosphorylated AKT, RAF, p70 S6 kinase, MEK, ERK and RSK1, showing the cells overcame AZD6244 inhibition of MEK by RTK activation of the ERK, AKT and mTOR pathways (FIG. 3G).

These findings indicate that targeted MEK inhibition significantly alters the activity of multiple kinases. It was therefore important to determine if the changes in kinase activity were specific for MEK inhibition or a function of growth inhibition. BEZ235 is a dual PI3K and mTOR inhibitor that strongly growth arrests SUM159 cells (FIG. S3E). BEZ235 inhibits p70 S6 kinase activity consistent with mTOR inhibition but has little effect on the ERK pathway (FIG. 10F). The SUM159 kinome responses to BEZ235 and AZD6244 were compared to determine if kinome reprogramming was inhibitor-specific or a function of growth arrest. Whereas AZD6244 induced PDGFRβ, VEGFR2 and AXL phosphorylation, BEZ235 treatment did not change the RTK phosphorylation profile except for an increase in phospho-AXL (FIG. 3H). MIB/MS profiles showed that AZD6244 altered the kinome profile differently from BEZ235, indicating that drug-induced kinome reprogramming is target specific (FIG. 10G).

### 6.5. MEK-ERK Inhibition Induces c-Myc Degradation Leading to RTK Reprogramming

ERK phosphorylates the transcription factor c-Myc at Ser62 and stabilizes the c-Myc protein by preventing its proteasomal degradation (Sears R, 2000). Sears et al. 2000 Genes Devel 14 2501-2514. Treatment of cells with AZD6244 results in the rapid loss of c-Myc protein and c-Myc mRNA (FIGS. 4A and B). Loss of phospho-c-Myc was transient, with return coinciding with ERK reactivation. However, total c-Myc protein and mRNA remained repressed in the continued presence of AZD6244, resulting in decreased Myc-Max heterodimerization that is required for Myc regulation of transcription (FIG. 4C). Marampon et al., 2006 Mol Cancer 5, 2-17; Wierstra and Alves 2008 The c-myc Promoter: Still MysterY and Challenge. In Advances in Cancer Research, F. V. W. George, and K. George, eds. (Academic Press), pp. 113-333. Even though MEK-ERK activation has recovered substantially after 24-72 h, total c-Myc expression remained repressed in the continued presence of AZD6244 (FIG. 4A-C).

c-Myc binds the promoter of human PDGFRβ (FIG. 11A) and represses expression of PDGFRβ. Oster et al. 2000 Mol Cell Biol 20 6768-6778. To define the role of c-Myc loss in the AZD6244 reprogramming response, RNAi techniques were applied to knockdown expression of c-Myc; the effect was to mimic the reprogrammed RTK and cytokine signature seen with AZD6244 treatment (FIG. 4D-F). Similar to the AZD6244 response, knockdown of c-Myc induced expression of PDGFRβ, VEGFR2 and PDGFB, and increased Tyr phosphorylation of PDGFRβ, VEGFR2, HER3 and AXL. RNAi knockdown of ERK1 and 2 was used to confirm that ERK inhibition was the primary signal inducing loss of c-Myc mRNA expression in the AZD6244 reprogramming of the kinome. Dual ERK1/2 knockdown resulted in reduced c-Myc and increased PDGFRβ expression (FIG. 11B). Thus, reprogramming of RTKs in response to AZD6244 occurs by loss of ERK-mediated stabilization of c-Myc and the subsequent transcriptional derepression of RTKs and cytokines that are negatively regulated by c-Myc. BEZ235 inhibition of mTOR and PI3K inhibits cell growth but does not change ERK activity, c-Myc expression (FIG. 11C) or RTK reprogramming (FIG. 11G), confirming the specificity of MEK-ERK in controlling c-Myc levels.

In AZD6244-resistant SUM159-R cells grown continuously in 5 μM AZD6244, c-Myc protein and RNA levels have partially returned because of the increased ERK activity stabilizing c-Myc (FIG. 4G-4I). This correlates with SUM159-R cells having an increased growth rate compared to cells acutely treated with MEK inhibitor (FIG. 3F). The level of c-Myc protein, however, is insufficient to completely repress RTK expression, which remains elevated compared to control cells, but at lower levels than that seen with 24-72 h AZD6244 treatment (FIGS. 4G and 4I). Increasing the concentration of AZD6244 5-fold to 25 μM inhibited ERK activation in SUM159-R cells (FIG. 4J), because the high dose of MEK inhibitor is able to more effectively inhibit RTK-stimulated reactivation of MEK-ERK signaling. The result is loss of phospho-c-Myc and total c-Myc protein and corresponding increases in RTK expression, similar to what was seen with control SUM159 cells treated with 5 μM AZD6244 (FIG. 4J). At the 25 μM dose of AZD6244, SUM159-R cells were growth arrested with loss of cyclin B1 and A2 expression, similar to acutely treated control SUM159 cells. In contrast, washout of AZD6244 from SUM159-R cell cultures increased ERK activity, stabilization of c-Myc expression and repression of RTK expression (FIGS. 4K and 4L). Thus, removal of the MEK inhibitor completely reversed kinome reprogramming (FIG. 4L and FIG. 11D).

Proteasomal degradation of c-Myc lacking phosphorylation at Ser62 triggers AZD6244-induced kinome reprogramming Treatment of cells with bortezomib, a proteasome inhibitor used clinically for myeloma, prevented AZD6244-induced c-Myc degradation (FIG. 4M) and loss of c-Myc RNA (FIGS. 11E and 11F). Bortezomib also inhibited upregulation of RTKs (FIGS. 4M and 4N) and readily reversed RTK expression in SUM159-R cells (FIGS. 4O and 4P). These findings show that AZD6244-induced c-Myc proteasomal degradation is responsible for kinome reprogramming and RTK upregulation. Stabilizing c-Myc by AZD6244 washout or proteasome inhibition prevented the onset of resistance to AZD6244 and reversed the resistance of SUM159-R cells. However, in the continued presence of AZD6244 ERK reactivation seems insufficient to fully reverse RTK reprogramming, suggesting ERK may not be fully reactivated. This was shown by measuring the phosphorylation of two ERK substrates, RSK1 and c-Myc, after only 1 h of AZD6244 washout from SUM159-R cells (FIG. 4Q). Phosphorylation of both substrates and phospho-ERK was increased, demonstrating the further activation of ERK with removal of the MEK inhibitor. Taken together, these data indicate persistent AZD6244-mediated suppression of MEK-ERK signaling despite a return of ERK1/2 phosphorylation with prolonged AZD6244 treatment.

### 6.6. RTK Reprogramming Rescues Cells from AZD6244-Induced Growth Arrest

RNAi knockdown of PDGFRβ in SUM159 cells resulted in increased growth inhibition in response to MEK inhibition (FIG. 5A), indicating the induction of RTK signaling was critical for survival and escape of cells growth inhibited by AZD6244. To test the role of additional RTKs in the rescue response of cells to MEK inhibition, siRNA knockdowns were performed for RTKs found to be transcriptionally induced and/or Tyr phosphorylated in response to U0126 in SUM159 (FIG. 5B) and MDA-MB-231 cells (FIG. 12A). As controls siRNA was used to knockdown BRAF, RAF1, ERK1 and 2; knockdown of each pathway member enhanced growth arrest observed with MEK inhibition (FIGS. 12A and 12B). Knockdown of PI3K and AKT produced a greater growth arrest response in SUM159 than MDA-MB-231 cells, consistent with a mutant PI3K being a driver in SUM159 cells. siRNA knockdown of Lyn and EPHA2 had no effect on the growth of either cell type in the presence or absence of MEK inhibitor (both Lyn and EPHA2 show a loss of MIB binding in response to AZD6244 or U0126, indicating inhibition of their activity). Knockdown of HER2 or HER3 had little effect in SUM159 cells with HER3 having a very modest effect in MDA-MB-231 cells. In contrast, knockdown of AXL, DDR1, DDR2, PDGFRβ and VEGFR2 each resulted in a synthetic lethal-like effect in the presence of U0126. That is, loss of RTK expression produced little growth inhibition by itself but synergistically inhibited growth in the presence of MEK inhibitor. Thus, loss of MEK-ERK signaling initiates a response involving multiple RTKs with each playing some role in subverting the effect of MEK inhibition.

### 6.7. AZD6244 in Combination with RTK Inhibitors

Our results suggested RTK inhibitors in combination with AZD6244 could block the growth promoting activity of the reprogrammed kinome. Given the repertoire of AZD6244-activated RTKs, sorafenib and foretinib were tested as single agents or in combination with AZD6244 for their ability to inhibit cell growth (FIGS. 5C and 5D). Where the two RTK inhibitors were ineffective as single agents, both were synergistic in inhibiting cell growth in combination with AZD6244, with sorafenib being most effective. Cell counting assays reinforced the strong synergistic growth arrest of the AZD6244+sorafenib combination with SUM159 cells (FIG. 5E); RTK arrays validated that sorafenib inhibited Tyr phosphorylation of multiple RTKs induced by AZD6244 (FIG. 5F). The combination of AZD6244+sorafenib enhanced the inhibition of ERK1/2, promoted decreased cyclin D1 levels and increased expression of the pro-apoptotic Bim protein compared to AZD6244 alone, indicating the cells are primed for apoptosis (FIGS. 5G and 12H).

Sorafenib inhibits PDGFRα and β, VEGFR2, DDR1 and DDR2, but is also an inhibitor of BRAF and RAF. Therefore, the action of different RAF inhibitors in combination with AZD6244 was assayed to determine if the effect of sorafenib could be mimicked by other BRAF/RAF1 inhibitors (FIGS. 12F and 12G). In MDA-MB-231 cells, the paradoxical BRAF/RAF activation is not observed, probably due to the G464V activating mutation in BRAF. The RAF inhibitor SB590885 inhibited MDA-MB-231 proliferation in combination with AZD6244 to a similar extent as sorafenib, consistent with the role of BRAF activation downstream of the observed RTK reprogramming as a driver of the proliferation. RAF inhibitors in combination with AZD6244 actually stimulated the growth of SUM159 cells (FIG. 12F), consistent with activation of wild type RAF signaling by both PLX4720 and SB590885. Sorafenib at low doses did not significantly stimulate proliferation, although these findings suggest there may be a weak enhancement of growth. At the highest dose tested, sorafenib synergistically inhibited growth of SUM159 cells in combination with AZD6244. Thus, sorafenib in combination with AZD6244 is able to inhibit growth of SUM159 cells more effectively than BRAF inhibitors by targeting both RTKs and RAF kinases.

SUM159-R cells that have become resistant to AZD6244 rely on RTK-driven reactivation of ERK for drug resistance. If a ten-fold higher dose of AZD6244 (50 μM) is used, ERK activity can be inhibited (FIG. 5G). At the lower dose of 5 μM AZD6244 that was used to develop the resistant SUM159-R cells, the addition of sorafenib inhibited ERK activity and cell growth (FIG. 5H). This finding is important because it indicates that AZD6244-induced activation of the upstream RTK-RAF pathway is driving ERK activation, and sorafenib can inhibit the RTK signals to prevent reactivation of ERK. In SUM159-R cells the combination of low therapeutic doses of AZD6244 and sorafenib is similarly effective as high dose AZD6244 at inhibiting ERK activation and cell growth (FIGS. 5H and 5I).

### 6.8. Kinome Reprogramming in the C3Tag TNBC GEMM

The genetically engineered C3Tag mouse model has a gene expression signature similar to human TNBC. Tumor tissue was harvested before or after oral delivery of AZD6244 for various times in the mouse. FIGS. 6A and 13 show the increased expression of PDGFRβ in response to AZD6244 in both the tumor cells and stroma of C3Tag breast cancers, demonstrating in vivo induction of PDGFRβ. FIG. 6B shows that a tumor-derived C3Tag breast cancer cell line responds to AZD6244 with upregulation of PDGFRβ and DDR RTKs, confirming the tumor cell response to MEK inhibitor.

We then profiled the ERK pathway after treating the C3Tag mice with AZD6244 or sorafenib (FIG. 6C). In response to both drugs, tumor MEK and ERK were strongly activated compared to untreated animals. With AZD6244 treatment, expression of RTKs was elevated over levels seen in tumors from untreated mice, similar to that seen with SUM159 cells. With sorafenib, the C3Tag tumors have a paradoxical ERK activation but do not alter RTK expression. FIG. 6D compares PDGFRβ and c-Myc expression following treatment of mice with AZD6244. In response to AZD6244, ERK-mediated phosphorylation of c-Myc at Ser62 is lost after 2 and 7 days of treatment, promoting degradation of c-Myc; with loss of c-Myc, PDGFRβ expression is strongly induced.

We then used MIB/MS to compare the kinome profile of C3Tag tumors from mice treated with AZD6244 versus sorafenib (FIG. 6E). The MIB kinase binding signatures for the two inhibitors overlap but exhibit significant differences, demonstrating selective reprogramming of the kinome. The MIB/MS profile shows the escape of MEK2 and ERK1 from AZD6244 inhibition, as was shown in SUM159 cells (FIGS. 6F and 2E). Sorafenib treatment decreased MIB binding of the previously reported sorafenib targets BRAF, PDGFRβ, CSF1R, DDR1, DDR2, KIT, MLTK and FRK. Karaman et al. 2008 Nat Biotech 26 127-132.

### 6.9. AZD6244 Plus Sorafenib Causes Tumor Regression

The response of C3Tag tumors to AZD6244 and sorafenib alone and in combination was determined (FIG. 7A). After only 2 days of AZD6244 or sorafenib treatment, the expression of VEGFR2 and PDFGRβ was increased. At the same time, increased phosphorylation of RAF at Ser338 is observed, demonstrating RAF activation. ERK was activated in response to single-agent AZD6244 or sorafenib, but this response was strongly inhibited by the combination of AZD6244 and sorafenib. The response to the individual agents was sustained at 7 days of treatment, although the sorafenib-induced increase in VEGFR2 and PDGFRβ appeared transient. The induction of RTKs in response to sorafenib was not seen with SUM159 cells and may be due to an early but transient inhibition of ERK activity in the C3Tag tumors. Just as in SUM159 and MDA-MB-231 cells, MEK inhibition caused loss of c-Myc in the C3Tag tumors (FIG. 7B). Whereas treatment with AZD6244 induced expression and activation of PDGFRβ, treatment of animals with the combination of AZD6244 and sorafenib inhibited both PDGFRβ and ERK phosphorylation (FIG. 7C). FIG. 7D shows the AZD6244 response in the tumor-derived C3Tag breast cancer cell line, revealing induction of PDGFRβ and loss of c-Myc. The combination of AZD6244 and sorafenib blocked ERK activation, enhancing c-Myc degradation, and decreased expression of PDGFRβ and cyclin B1. Cotreatment with AZD6244 and sorafenib also caused a strong growth arrest in the C3Tag breast cancer cell line (FIG. 7E).

Our findings showed that the combination of AZD6244 and sorafenib was significantly more effective in inhibiting ERK activation in 2 and 7 day treated C3Tag mice and the C3Tag tumor cell line. Therefore, C3Tag mice were allowed to develop tumors and then treated for 21 days with AZD6244 or sorafenib, alone or in combination (FIG. 7F and FIG. 14A). Sorafenib treatment had no effect on tumor progression, whereas 30 percent of the AZD treated mice showed significant tumor regression. In contrast, 77 percent of mice treated with AZD6244 and sorafenib had tumor regression, demonstrating a significantly greater effect of the combination therapy versus AZD6244 alone. TUNEL assays of the tumors showed that the combination of AZD6244 and sorafenib induced a strong apoptotic response in only 2 days of treatment, contrasting with single drug treatment (FIG. 7G and FIG. 14B).

### 6.10. Discussion

A novel approach to study the reprogramming of protein kinase networks “en masse” is described. The methods allowed the isolation and analysis of protein kinases from cells and tumors with ˜50% of the expressed kinome assayed in a single mass spectrometry run. Profiling MIB binding of kinases is a highly sensitive method to simultaneously monitor activation and inhibition of numerous kinases. This profiling technique allows interrogation of kinases known by sequence but which have been understudied due to lack of biologic or phenotypic knowledge or reagent availability. An example of the latter is the ability to distinguish changes in MEK1 and MEK2.

This technique identified a kinome response signature to the selective MEK1/2 kinase inhibitor AZD6244. The only defined substrates for MEK are ERK1 and 2, yet changes in activity of kinases in every subfamily of the kinome was observed in response to MEK inhibition. Kinome assessment showed a time-dependent reprogramming that involved an early loss of ERK feedback regulation of RAF and MEK, as well as increased MKP3 protein stability. The increased expression of MKP3 functions to enhance ERK inactivation. In contrast, the loss of RAF and MEK feedback inhibition would allow upstream activation of the pathway. The time-dependent change in MIB binding of specific RTKs such as PDGFRβ and DDR1 was readily detected and provided the critical experimental observation that MEK inhibition was driving the expression and activation of multiple RTKs, each of which are capable of stimulating the RAF-MEK-ERK pathway. Importantly, c-Myc degradation was identified as a key mechanism mediating kinome reprogramming; preventing proteasomal degradation of hypophosphorylated c-Myc inhibited the reprogramming response. RNAi knockdown of ERK or c-Myc recapitulated the MEK inhibitor induced expression and Tyr phosphorylation of several RTKs, demonstrating ERK regulation of c-Myc stability is critical in controlling the expression and activation of specific kinases. The fact that multiple RTKs are activated in response to MEK inhibition demonstrates the difficulty in using single kinase inhibitors to arrest tumor progression.

### 6.11. MEK2 Escapes Inhibition

MIB binding coupled with quantitative mass spectrometry is a very sensitive and selective method with which to measure the global effects of kinase inhibition; that is particularly important for kinases that have been traditionally understudied or for which reagents are not available. Analysis of the ERK pathway of cells treated with AZD6244 showed a time-dependent rescue of BRAF/RAF, MEK2, ERK1 and RSK1 binding to MIBs. MIB binding of these kinases was demonstrated to be a function of their activation. The time course of recovery is similar to that of AZD6244-induced RTK expression. The C3Tag tumor shows a similar increase in MEK2 and ERK1 binding after AZD6244 treatment, mimicking the reprogramming response observed in SUM159 cells. Published work with a similar MEK inhibitor, GSK1120212, which binds to the MEK allosteric regulatory site (as does AZD6244) provides insight into how MEK2 escapes inhibition. Gilmartin et al. 2011 Clin Cancer Res 17 989-1000. MEK phosphorylated at the activation loop serines has a 20-fold lower affinity for GSK1120212 than nonphosphorylated MEK, effectively alleviating allosteric site inhibition of MEK. Because ERK activity is increasing over time, MEK1 would be feedback phosphorylated at its negative regulatory site Thr292, preventing MEK1 reactivation even in the setting of RTK reprogramming; MEK2, however, lacks this regulatory site and selectively escapes inhibition. This suggests a unique paradigm of activation of an upstream signaling pathway increasing the IC50 of an inhibitor for a target kinase, a paradigm that would have been difficult to detect with current reagents.

### 6.12. Rational Design of Combination Therapies

In many tumor types Tyr kinases are molecular drivers of transformation and also play a major role in resistance to therapy. In one form of breast cancer it was demonstrated that the tumor response to targeted kinase inhibition involves the induction and/or activation of multiple RTKs that contribute to drug resistance. Claudin-low SUM159 cells and the C3-Tag breast cancer GEMM were remarkably similar in response to AZD6244 with induction and activation of PDGFRβ, VEGFR2, CSFR1, DDR1/DDR2 and AXL. The claudin-low MDA-MB-231 cell line was somewhat less responsive, but still showed the induction of PDGFRβ, DDR1, and DDR2 and activation of AXL with AZD6244 treatment. RNAi knockdown of the different RTKs indicated that each kinase contributed to the survival response in SUM159 and MDA-MB-231 cells. Given the repertoire of RTKs whose expression and activity is induced with AZD6244 treatment, the combination therapy of sorafenib and AZD6244 was predicted to “broaden” the kinase targeting sufficiently to produce significant therapeutic benefit. The combination therapy increased apoptosis and tumor regression significantly compared to either drug alone in the C3Tag TBNC GEMM.

AZD6244-induced RTKs (and Ser/Thr kinases) were identified using a combination of MIB/MS and immunoblotting of cell lines and C3Tag tumors. A signature of therapeutic response resistance was created thus allowing a rational prediction of combinatorial therapies. The approach can be extended to human tumors using so-called “window trials” in which a patient is treated with a targeted agent prior to surgery and their tumor analyzed at excision for kinome-resistance signatures. Importantly, the kinome response has been shown to be unique for inhibitors targeting different kinases. The response of different tumor types to a common inhibitor may also vary. Thus, this systems kinome approach can be applied to help define patterns of resistance for a variety of drugs and biopsy-accessible tumor types.

### 6.13. Experimental Procedures

Cell Culture: MDA-MB-231 cells were grown in DMEM/F12 supplemented with 10% FBS. SUM-159 cells were grown in DMEM/F12 supplemented with 10% FBS 1 μg/mL hydrocortisone, and 5 μg/mL insulin. SUM159-R cells were continually grown in the presence of 5 μM AZD6244. For SILAC labeling, cells were grown for five doublings in arginine- and lysine-depleted media (as above) supplemented with either unlabeled L-arginine (42 mg/L) and L-lysine (71 mg/L) or equimolar amounts of heavy isotope labeled [13C6,15N4]arginine (Arg10) and [13C6]lysine (Lys6) (Cambridge Isotope Laboratories). Proliferation was quantified using Cell-Titer Glo Luminescent Cell Viability Assay (Promega). Fresh media containing DMSO or kinase inhibitors was added daily and experiments were performed in triplicate.

Multiplexed inhibitor bead affinity chromatography: Cells and tumors were lysed and harvested as previously described (Oppermann et al., 2009). Oppermann et al., 2009 Mol Cell Proteo 8 1751-1764. Briefly, lysates were brought to 1 M NaCl and passed through columns of washed inhibitor-conjugated beads (bisindoylmaleimide-X, SB203580, lapatinib, dasatinib, purvalanol B, VI16832, PP58) to isolate protein kinases from the lysates (see Supplemental Experimental Procedures for MIBs preparation). Kinase-bound inhibitor beads were washed with high-salt buffer and 0.1% SDS before elution in 0.5% SDS solution in high heat. Proteins were purified using chloroform/methanol extraction, resuspended in 50 mM ammonium bicarbonate (pH 8.0) or 50 mM HEPES (pH 8.0) for SILAC or iTRAQ respectfully. Samples were digested overnight at 37° C. with sequencing grade modified trypsin (Promega). iTRAQ labeling of digested peptides was carried out using iTRAQ 4-plex reagent (AB SCIEX) for 2 hrs at room temperature in the dark. Peptides were dried down, separated using Strong Cation Exchange Spin Columns, Mini and isolated with PepClean C-18 Spin Columns (Thermo Scientific).

LC-MS/MS analysis: MS and MS/MS data were acquired using a MALDI TOF/TOF 5800 (AB SCIEX). Peptides were analyzed using ProteinPilot Software Version 3.0 (AB SCIEX) and identification using UniProtKB/Swiss-Prot database (release Oct. 15, 2009). Proteins were only accepted when at least 1 unique peptide was identified at 99% confidence. ProteinPilot software 3.0 identified and quantified changes in kinase binding to MIBs utilizing the Pro Group Algorithm. Quant ratios are corrected for bias due to unequal mixing during the combination of the different labeled samples, under the assumption that most proteins do not change in expression. For each protein ratio reported, a p-value is computed based on Student's t-distribution under the null hypothesis that the protein ratio is 1. MIB/MS analysis with cell lines was done in 2-3 independent experiments. A set of three independent experiments using SILAC labeled SUM159 cells treated with AZD6244 or DMSO was used to assess statistical significance and reproducibility of MIBs/MS to profile kinome response (Supplementary Methods).

Western blotting and RTK arrays: Western blotting and RTK array analysis were performed as previously described (Amin et al., 2010). Amin et al. 2010 Sci Transl Med 2, 16ra17. Detailed methods and antibodies are provided in Supplemental Experimental Procedures.

qRT-PCR: Total RNA was isolated from human breast cancer cell lines or murine tumors using the RNeasy® Plus Mini Kit (Qiagen). Real-time RT-PCR was performed on diluted cDNA using the Applied Biosystems 7500 Fast Real-Time PCR System (standard program) and inventoried TaqMan® Gene Expression Assays. Each cDNA sample was assayed in triplicate. Fold change with respect to the calibrator represents the average of the triplicate values, with error bars representing the range of the mean (95% confidence).

In vivo tumorigenesis experiments: Animal handling and procedures were approved by the University of North Carolina at Chapel Hill Institutional Animal Care and Use Committee and followed the NIH guidelines. Male C3Tag mice were bred with wild type females to produce experimental offspring. Mice were examined for tumors weekly until a palpable mass was found. Treatment began the same day. Tumor size was assessed twice weekly by caliper measurements of tumor areas ((width)2×length))/2 for 21 days. Percent change of tumor volume was calculated using (Final volume−Initial Volume)/Initial Volume and graphed using R (http://www.r-project.org/). Drugs were incorporated into the diet of mice to achieve a daily dose of AZD6244 20 mg/kg and sorafenib 30 mg/kg. Food was provided ab libitum and the amount of daily food intake was pre-determined using Jackson Labs Phenome Database. Tumors at harvest were cut in half and either snap-frozen in liquid nitrogen and stored at −80° C. or placed in neutral buffered 10% formalin solution.

Human breast tissue procurement: All human breast tissue was obtained from the Tissue Procurement Facility in compliance with the laws and institutional guidelines as approved by the University of North Carolina at Chapel Hill IRB committee. Clinical specimens were molecularly phenotyped by gene expression analysis performed in the laboratory of Dr. Charles M. Perou.

### 6.14. Supplementary Data and Experiments

Supplemental data includes Supplemental Experimental Procedures, 5 tables and seven figures.

Cell Culture: Myl CML cells were cultured in RPMI 1640 medium (Invitrogen, Carlsbad, Calif.) supplemented with 10% fetal bovine serum (Atlanta Biologicals, Norcross, Ga.) and 1% antibiotic/antimycotic (Invitrogen). For SILAC labeling, cells were grown for five doublings in arginine- and lysine-depleted media (as above) supplemented with either unlabeled L-arginine (84 mg/L) and L-lysine (48 mg/L) or equimolar amounts of heavy isotope labeled [13C6,15N4]arginine (Arg10) and [13C6]lysine (Lys6) (Cambridge Isotope Laboratories) as described previously (Ong SE, 2002).

Generation of AZD6244 resistant SUM159 cells: SUM159 cells were cultured in DMEM/F12 supplemented with 10% FBS media containing 5 μM AZD6244. Media was changed every 2 days, maintaining inhibitor concentrations of 5 μM AZD6244.

Generation of immortalized cell line from a C3Tag tumor. An autochthonous tumor from a C3Tag mouse was excised and dissociated in a sterile fashion in the presence of 0.25% trypsin (Gibco). Cells were then passed through a 40 micron cell strainer and grown in the presence of DMEM+10% FBS. Cells were isolated and expression of SV40T antigen verified by immunoblotting with antibodies specific to SV40 large T (EMD Biosciences, monoclonal, clone PAb416).

### 6.15. Compounds

Sorafenib, U0126 and bortezomib were purchased from LC Labs (Woburn, Mass.). BEZ235 was purchased from Selleck (Houston, Tex.), Bisindolylmaleimide-X was from Alexis (Enzo Life Sci. Farmingdale, N.Y.) and Purvalanol B was from Tocris (Bristol United Kingdom). Foretinib and AZD6244 were synthesized according to the procedures described in two patent applications (WO2005030140A2, WO2007002157A2). PP58 (Klutchko et al., 1998 J Med Chem 41 3276-3292), VI16832 (Daub et al., 2008) Dasatinib (Das et al., 2006 J Med Chem 49 6819-6832), Lapatinib (Barker et al., 2001 Bioorg Med Chem Let 11 1911-1914), SB203580 (Gallagher et al., 1997 Bioorg Med Chem 5, 49-64). PLX4720 and SB590885 were custom synthesized according to previously described methods, specifically, PLX-4720, Tsai et al., 2008 Proc Natl Acad Sci USA. 105(8):3041-3046 and SB590885, King et al. 2006 Cancer Res. 66(23): 11100-5.

### 6.16. Synthesis of Affinity Ligands

### 6.16.1. General Procedures

HPLC spectra of all compounds were acquired from an Agilent 6110 Series system with UV detector set to 220 nm. Samples were injected (5 μL) onto an Agilent Eclipse Plus 4.6×50 mm, 1.8 μM, C18 column at room temperature. A linear gradient from 10% to 100% B (MeOH+0.1% Acetic Acid) in 5 0 min was followed by pumping 100% B for another 2 minutes with A being H2O+0.1% acetic acid. The flow rate was 1.0 mL/min. Mass spectra (MS) data were acquired in positive ion mode using an Agilent 6110 single quadrupole mass spectrometer with an electrospray ionization (ESI) source. High-resolution (positive ion) mass spectra (HRMS) were acquired using a Shimadzu LCMS-IT-T of time-of-flight mass spectrometer. Nuclear Magnetic Resonance (NMR) spectra were recorded at Varian Mercury spectrometer with 400 MHz for proton (1H NMR) and 100 MHz for carbon (13C NMR); chemical shifts are reported in ppm (δ).

### 6.16.2. Synthesis of (2-(6-(4-(3-aminopropyl)piperazin-1-yl)-2-methylpyrimidin-4-ylamino)thiazol-5-yl)(2-chloro-6-methylphenyl)methanone

### Preparation of (2-(6-(4-(3-aminopropyl)piperazin-1-yl)-2-methylpyrimidin-4-ylamino)thiazol-5-yl)(2-chloro-6-methylphenyl)methanone

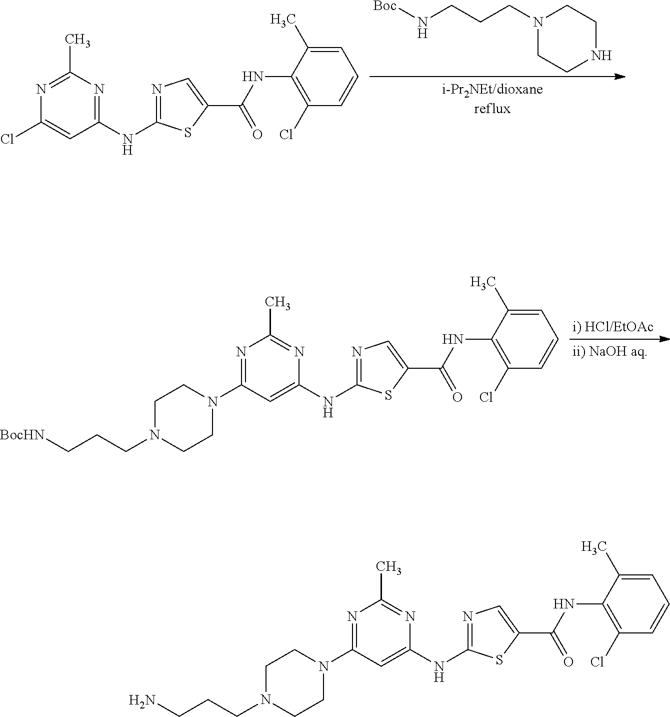 [SMILES: Cc1nc(Cl)cc(Nc2ncc(C(=O)Nc3c(C)cccc3Cl)s2)n1 ; CC(C)(C)OC(=O)NCCCN1CCNCC1 ; Cc1nc(Nc2ncc(C(=O)Nc3c(C)cccc3Cl)s2)cc(N2CCN(CCCNC(=O)OC(C)(C)C)CC2)n1 ; Cc1nc(Nc2ncc(C(=O)Nc3c(C)cccc3Cl)s2)cc(N2CCN(CCCN)CC2)n1]

To a solution of 573 mg (1.45 mmol) 2-(6-chloro-2-methylpyrimidin-4-ylamino)-N-(2-chloro-6-methylphenyl)thiazole-5-carboxamide (Das et al., 2006) in 20 mL dioxane was added 1.7 g (7.29 mmol) tert-butyl 3-(piperazin-1-yl)propylcarbamate and 0.58 mL (2.75 mmol) N-ethyldiisopropylamine. The resulting mixture was refluxed overnight. Solvent was removed under reduced pressure and the residue was washed repeatedly with ether and filtered to give the crude product tert-butyl 3-(4-(6-(5-(2-chloro-6-methylphenylcarbamoyl)thiazol-2-ylamino)-2-methylpyrimidin-4-yl)piperazin-1-yl)propylcarbamate as pale yellow solid which was used for the next reaction without further purification. The obtained solid was dissolved in 5 mL ethyl acetate. To that solution was added drop wise the solution of hydrochloride in ethyl acetate over 30 min at 0° C. with vigorous stirring. The precipitate was collected by filtration and washed repeatedly with ether. After dried in vacuum, the solid was portioned between 30 mL aqueous sodium hydroxide and 30 mL ethyl acetate. The separated water phase was extracted two times with 30 mL ethyl acetate. The collected organic phase was washed with brine, dried with magnesium sulphate, and the solvent was evaporated under reduced pressure. The residue was purified by silica gel column chromatography to give the pure product.

Yield 462 mg (75%)

HPLC 99%, Rt: 3.670 min; Mol. Mass. 501 [M+1]⁺; HRMS (ESI) calculated for C₂₃H₃₀ClN₈OS [M+1]⁺, 501.1952, found 501.1938.

¹H NMR (400 MHz, CD₃OD) δ 8.18 (s, 1H), 7.34 (dd, J=7.1, 2.0 Hz, 1H), 7.27-7.19 (m, 2H), 6.31 (s, 1H), 4.02 (bs, 4H), 3.43 (s, 4H), 3.30-3.24 (m, 2H), 3.04 (t, J=8.0 Hz, 2H), 2.54 (s, 3H), 2.30 (s, 3H), 2.22-2.10 (m, 2H). ¹³C NMR (100 MHz, CD₃OD) δ 165.5, 163.6, 162.7, 161.5, 158.0, 140.5, 134.3, 134.2, 130.3, 130.0, 128.5, 85.7, 55.0, 52.7, 42.8, 38.0, 23.5, 18.8.

### 6.16.3. Preparation of 6-(2-aminoethoxy)-N-(3-chloro-4-fluorophenyl)-7-methoxyquinazolin-4-amine

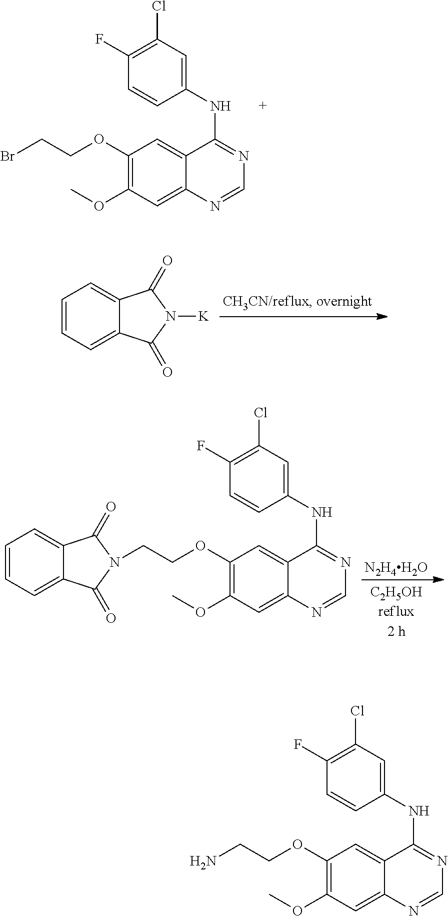

The solution of 6.4 g (15 mmol) 6-(2-bromoethoxy)-N-(3-chloro-4-fluorophenyl)-7-methoxyquinazolin-4-amine (Barker et al., 2001), 3.3 g (18 mmol) in 100 mL acetonitrile was refluxed overnight. The solvent was evaporated under reduced pressure, and the residue was re-dissolved in 100 mL ethanol. Ten 1.03 mL (18 mmol) hydrazine hydrate was added and the resulting mixture was stirred for 2 hours. Ethanol was removed under reduced pressure and the residue was purified by silica gel column chromatography to give the pure product.

Yield 3 g (56%)

HPLC 99%, Rt: 3.690 min; Mol. Mass. 363 [M+1]⁺; FIRMS (ESI) calculated for C₁₇H₁₇ClFN₄O₂ [M+1]⁺, 363.1024, found 363.1012.

¹H NMR (400 MHz, CD₃OD) δ 8.47 (s, 1H), 8.03 (dd, J=6.7, 2.6 Hz, 1H), 7.73 (s, 1H), 7.71-7.68 (m, 1H), 7.27 (t, J=9.0 Hz, 1H), 7.19 (s, 1H), 4.23 (t, J=6.7 Hz, 1H), 4.03 (s, 3H), 3.14 (t, J=6.7 Hz, 2H). ¹³C NMR (100 MHz, CD₃OD) δ 158.39, 157.16, 156.84, 154.73, 153.90, 150.44, 147.79, 137.48, 125.77, 123.84, 123.77, 121.44, 121.25, 117.47, 117.25, 111.41, 110.44, 107.32, 103.72, 72.04, 56.63, 41.73.

### 6.16.4. Preparation of 6-(2-aminoethoxy)-N-(3-chloro-4-(3-fluorobenzyloxy)phenyl)-7-methoxyquinazolin-4-amine

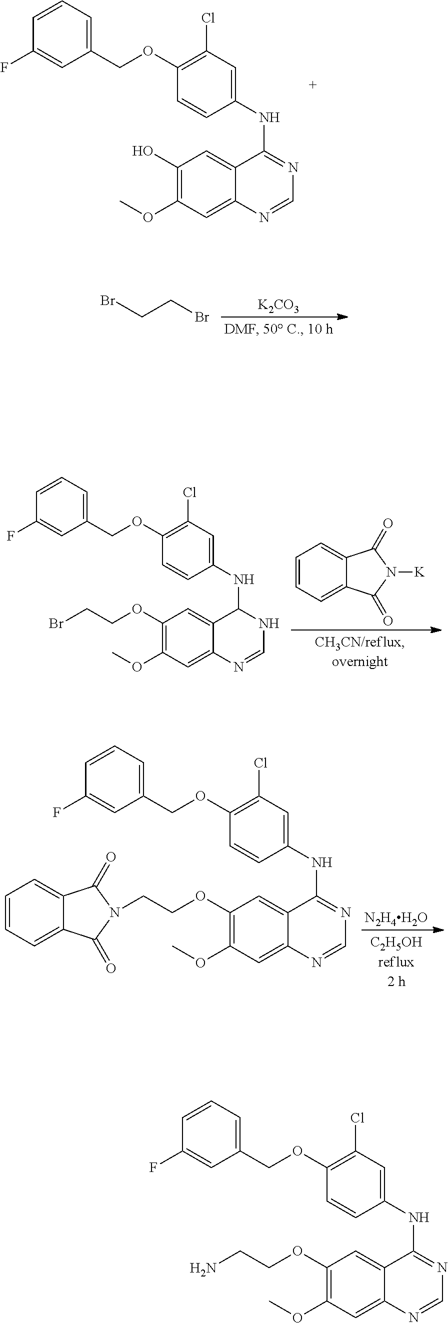

The solution of 10 g (24 mmol) 4-(3-chloro-4-(3-fluorobenzyloxy)phenylamino)-7-methoxyquinazolin-6-ol (Cai et al., 2010 J Med Chem 53, 2000-2009), 6.6 g (48 mmol) in 100 mL dimethylformamide was stirred at 50° C. for 10 hours. Water was added and the mixture was extracted three times with dichloromethane. The collected organic layer was washed with brine, dried with magnesium sulfate, and the solvent was evaporated under reduced pressure. The residue was purified by silica gel column chromatography to give the 5 g (11.8 mmol, yield 42%) compound 6-(2-bromoethoxy)-N-(3-chloro-4-(3-fluorobenzyloxy)phenyl)-7-methoxy-3,4-dihydroquinazolin-4-amine, which was then dissolved in 80 mL acetonitrile. To that solution was added 2.6 g (14 mmol) potassium 1,3-dioxoisoindolin-2-ide and the resulting mixture was refluxed overnight. The solvent was removed under reduced pressure and the residue was re-dissolved in 100 mL ethanol. Then 1.03 mL (18 mmol) hydrazine hydrate was added and the reaction mixture was stirred and refluxed for 2 hours. Ethanol was removed under reduced pressure and the residue was purified by silica gel column chromatography to give the pure product.

Yield 2.7 g (63%)

HPLC 99%, Rt: 4.516 min; Mol. Mass. 469 [M+1]⁺; HRMS (ESI) calculated for C₂₄H₂₃ClFN₄O₃ [M+1]⁺, 469.1443, found 469.1433.

¹H NMR (400 MHz, CD₃OD) δ 8.41 (d, J=1.0 Hz, 1H), 7.87 (dd, J=2.5, 1.0 Hz, 1H), 7.71 (s, 1H), 7.57 (ddd, J=8.9, 2.6, 1.0 Hz, 1H), 7.45-7.40 (m, 1H), 7.33 (d, J=7.6 Hz, 1H), 7.27 (d, J=9.8 Hz, 1H), 7.18-7.12 (m, 2H), 7.10-7.05 (m, 1H), 5.22 (s, 2H), 4.22 (t, J=5.1 Hz, 1H), 4.02 (s, 3H), 3.14 (t, J=5.1 Hz, 2H). ¹³C NMR (100 MHz, CD₃OD) δ 165.61, 163.18, 158.59, 156.72, 154.01, 152.20, 150.29, 147.62, 141.23, 141.16, 134.36, 131.38, 131.29, 126.32, 123.93, 123.90, 123.87, 123.79, 115.67, 115.46, 115.44, 114.99, 114.77, 110.38, 107.29, 103.87, 71.92, 71.22, 71.20, 56.60, 41.70.

### 6.16.5. 2,4, Diaminopyrimidine, Pyrazole Ligand Synthesis

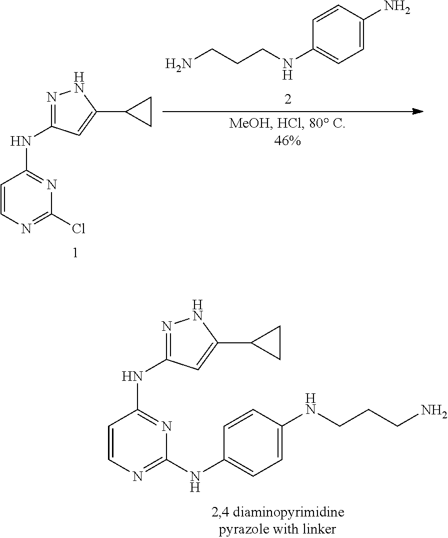 [SMILES: NCCCNc1ccc(N)cc1 ; Clc1nccc(Nc2cc(C3CC3)[nH]n2)n1 ; NCCCNc1ccc(Nc2nccc(Nc3cc(C4CC4)[nH]n3)n2)cc1]

Intermediate 1 was prepared according to the procedures described in J. Am. Chem. Soc., 2008, 130 (51), 17568-17574. Intermediate 2 was prepared according to the procedures described in GB 966083/U.S. Pat. No. 3,198,763.

Procedure: A solution of Intermediate 1 (460 mg, 1.95 mmol), Intermediate 2 (668 mg, 4 0 mmol), and conc. HCl (30 drops) in 4 mL of MeOH was heated at 80° C. by microwave irradiation for 40 min. The solution was cooled to room temperature and filtered. The solid was washed with MeOH to give the desired 2,4 diaminopyrimindine pyrazole compound (364 mg, 46%).

¹H NMR (300 MHz, DMSO): δ ppm 8.13 (br, 1H), 7.89 (br, 1H), 7.41 (br, 2H), 7.16 (br, 2H), 6.44 (br, 1H), 3.62 (br, 2H), 2.92 (t, J=7.5 Hz, 2H), 1.97 (t, J=7.2 Hz, 2H), 1.92-1.87 (m, 1H), 0.97-0.93 (m, 2H), 0.60 (br, 2H). HPLC: 95%, RT 1.678 min. MS (ESI) m/z 365.0[M+H]⁺.

### 6.16.6. Synthesis of 3-(2-(5-chloro-7H-pyrrolo[2,3-d]pyrimidin-4-yl)-2,8-diazaspiro[4.5]decan-8-yl)propan-1-amine

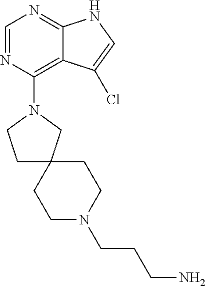 [SMILES: NCCCN1CCC2(CC1)CCN(c1ncnc3[nH]cc(Cl)c13)C2]

Synthetic Scheme:

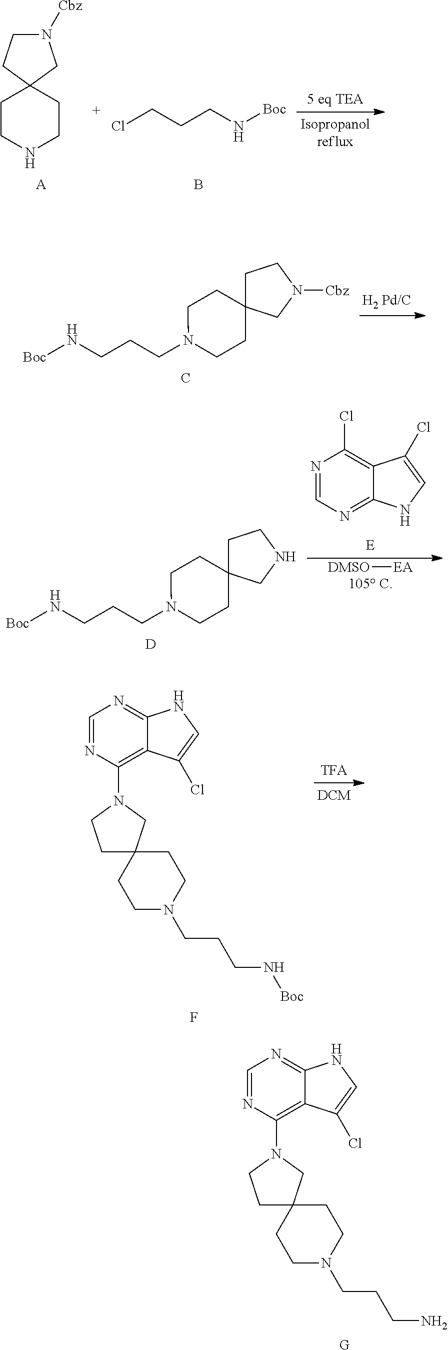

### Synthetic Procedures

### Benzyl 8-(3-(tert-butoxycarbonylamino)propyl)-2,8-diazaspiro[4.5]decane-2-carboxylate (C)

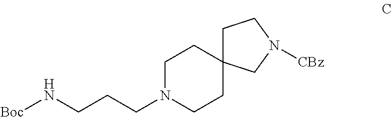

To the solution of Benzyl 2,8-diazaspiro[4.5]decane-2-carboxylate (A, 1.1 g, 4.01 mmol) and tert-butyl 3-chloropropy-lcarbamate (B, 1.16 g, 6.01 mmol) in isopropanol (30 mL) was added TEA (2.0 mL, 13.8 mmol). The mixture was heated to reflux for 20 h until TLC indicated A had been consumed (DCM:MeOH, 10:1, Chromogenic reagent Ninhydrin). The isopropanol was removed under reduced pressure, and the residue was purified by flash column (silica gel, DCM:MeOH, 30:1) to provide C as a soft white solid (600 mg, 35%).

### tert-Butyl 3-(2,8-diazaspiro[4.5]decan-8-yl)propylcarbamate (D)

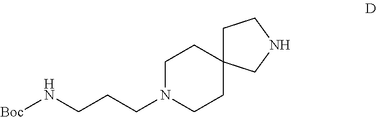 [SMILES: CC(C)(C)OC(=O)NCCCN1CCC2(CCNC2)CC1]

Benzyl 8-(3-(tert-butoxycarbonylamino)propyl)-2,8-diazaspiro[4.5]decane-2-carboxylate (C, 580 mg, 1.34 mmol) was dissolved in MeOH (10 mL). The solution was purged with vacuum/nitrogen cycles (×3) before the addition of Pd/C (140 mg, 24%) under vacuum. And the mixture was purged with vacuum/Hydrogen cycles (×3). Then the resulting mixture was stirred under H₂ for 6 h. The mixture was filtered through Celite and the filtrate was concentrated in vacuo to provide D as a white solid 490 mg that was used directly in the next step without further purification.

### tert-Butyl 3-(2-(5-chloro-7H-pyrrolo[2,3-d]pyrimidin-4-yl)-2,8-diazaspiro[4.5]decan-8-yl)propylcarbamate (F)

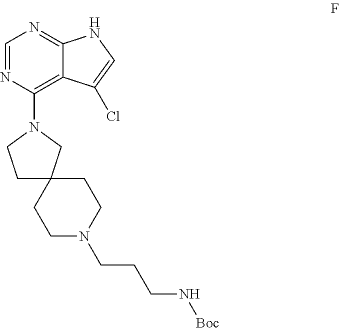 [SMILES: CC(C)(C)OC(=O)NCCCN1CCC2(CC1)CCN(c1ncnc3[nH]cc(Cl)c13)C2]

To a solution of 4,5-dichloro-7H-pyrrolo[2,3-d]pyrimidine (E, 271 mg, 1.44 mmol) and tert-butyl 3-(2,8-diazaspiro[4.5]decan-8-yl)propylcarbamate (D, 472 mg, 1.59 mmol) in DMSO (5 mL) was added DIPEA (0.5 mL, 2.88 mmol), followed by Ethyl Acetate (5 mL). The resulting reaction mixture was stirred overnight at 102° C. until TLC indicated E had been consumed (DCM:MeOH, 10:1). The reaction mixture was cooled to rt, diluted with H₂O (60 mL), extracted with DCM (25 mL×3), dried over Na₂SO₄, concentrated in vacuo to give the residue which was purified via a flash column (silica gel, DCM:MeOH, 30:1) to provide F as a yellowish solid (180 mg, 28%). ¹H NMR (300 MHz, CDCl3): δ 11.597 (1H, brs), 8.284 (1H, s), 7.120 (1H, s), 5.587 (1H, brs), 3.950 (2H, t, J=7.2 Hz), 3.730 (2H, s), 3.205 (2H, d, J=5.7 Hz), 2.623-2.459 (6H, m), 1.885 (2H, t, J=7.2 Hz), 1.782-1.642 (6H, m), 1.446 (9H, s).

### 3-(2-(5-Chloro-7H-pyrrolo[2,3-d]pyrimidin-4-yl)-2,8-diazaspiro[4.5]decan-8-yl)propan-1-amine (G)

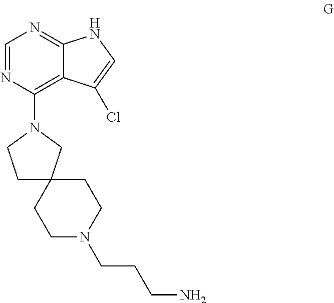 [SMILES: NCCCN1CCC2(CC1)CCN(c1ncnc3[nH]cc(Cl)c13)C2]

To a solution of tert-butyl 3-(2-(5-chloro-7H-pyrrolo[2,3-d]pyrimidin-4-yl)-2,8-diazaspiro[4.5]decan-8-yl)propylcarbamate (F, 198 mg, 0.44 mmol) in DCM (5 mL) was added TFA (0.5 mL). The resulting reaction mixture was stirred at room temperature until TLC indicated F had been consumed (DCM:MeOH, 10:1). Solvent was removed and residue was purified by HPLC to give the desired compound in TFA salt. After washing with saturated aqueous Na₂CO₃ solution and extracted with DCM, final compound G was obtained as yellow solid (85 mg, 55%). ¹H NMR (400 MHz, cd₃od) δ 8.08 (s, 1H), 7.17 (s, 1H), 3.89 (t, J=7.1 Hz, 2H), 3.71 (s, 2H), 3.34 (s, 1H), 2.99 (t, J=6.9 Hz, 2H), 2.61 (bs, 2H), 2.53 (t, J=6.8 Hz, 2H), 2.43 (bs, 2H), 1.94-1.88 (m, 2H), 1.82 (p, J=6.9 Hz, 2H), 1.68 (t, J=5.6 Hz, 4H).

### 6.16.7. Synthesis of N-(2-(4-((8-bicyclo[2.2.1]heptan-2-yl)-7-oxo-7,8-dihydropyrido[2,3-d]pyrimidin-2-yl)amino)phenoxy)ethyl)-5-(3aS,4S,6aR)-2-oxohexahydro-1H-thieno[3,4-d]imidazol-4-yl)pentanamide

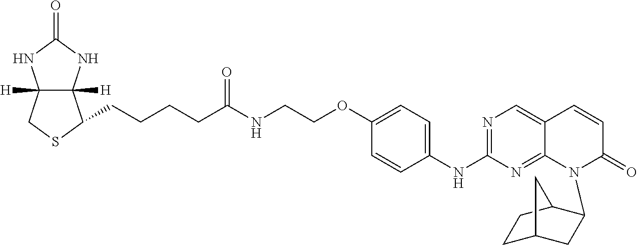 [SMILES: O=C(CCCC[C@@H]1SC[C@@H]2NC(=O)N[C@@H]21)NCCOc1ccc(Nc2ncc3ccc(=O)n(C4CC5CCC4C5)c3n2)cc1]

To a solution of VI16832 (83 mg, 0.212 mmol) (prepared according to published procedures (Daub et al., Mol. Cell, 2008, 31, 438-448) and Biotin (78 mg, 0.318 mmol) in DMF (1.0 mL) was added DMAP (5 mg, 0.041 mmol) and N-(3-Dimethylaminopropyl)-N′-ethylcarbodiimide hydrochloride (EDC, 81 mg, 0.424 mmol) at 0° C. The resulting mixture was stirred overnight at rt and purified by preparative HPLC to give the title compound as a yellow solid (96 mg, 73%). ¹H NMR (400 MHz, MeOH-d4) δ 8.58 (s, 1H), 7.65 (d, J=9.3 Hz, 1H), 7.60-7.51 (m, 2H), 7.00-6.89 (m, 2H), 6.29 (d, J=9.3 Hz, 1H), 5.34 (t, J=7.9 Hz, 1H), 4.41 (dd, J=7.9, 4.3 Hz, 1H), 4.19 (dd, J=7.9, 4.5 Hz, 1H), 4.05 (t, J=5.4 Hz, 2H), 3.62-3.52 (m, 2H), 3.11-3.03 (m, 1H), 2.85 (dd, J=12.7, 5.0 Hz, 1H), 2.69-2.57 (m, 2H), 2.50-2.43 (m, 1H), 2.42-2.34 (m, 1H), 2.24 (t, J=7.2 Hz, 2H), 2.20-2.11 (m, 1H), 1.83-1.50 (m, 7H), 1.48-1.34 (m, 3H), 1.33-1.20 (m, 2H). MS (ESI): 618 [M+H]⁺. HPLC: 100%, RT: 4.69 min.

### 6.16.8. Synthesis of N-(2-(4-((8-bicyclo[2.2.1]heptan-2-yl)-7-oxo-7,8-dihydropyrido[2,3-d]pyrimidin-2-yl)amino)phenoxy)ethyl)-1-(5-((3aS,4S,6aR)-2-oxohexahydro-1H-thieno[3,4-d]imidazol-4-yl)pentanamido)-3,6,9,12,15,18,21,24,27,30,33,36-dodecaoxanonatriacontan-39-amide

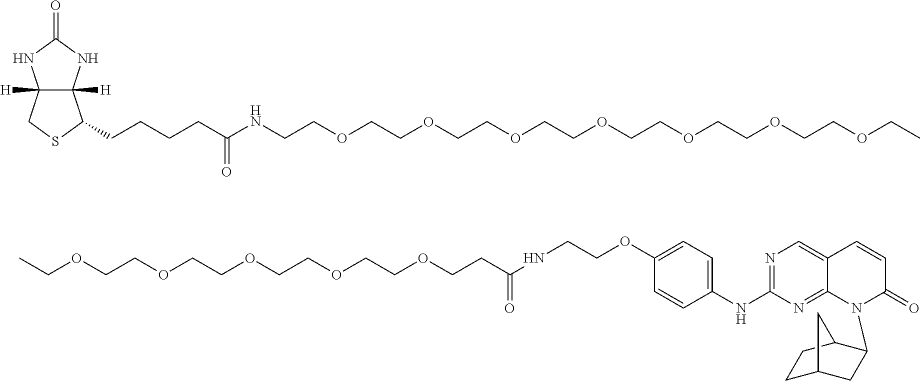

The desired compound was prepared following the procedures described for the above compound. The title compound was obtained as a yellow oil (46 mg, 62% yield). ¹H NMR (400 MHz, MeOH-d4) δ 8.61 (s, 1H), 7.68 (d, J=9.4 Hz, 1H), 7.60-7.53 (m, 2H), 7.02-6.94 (m, 2H), 6.33 (d, J=9.3 Hz, 1H), 5.34-5.27 (m, 1H), 4.49 (ddd, J=7.9, 4.9, 0.8 Hz, 1H), 4.30 (dd, J=7.9, 4.5 Hz, 1H), 4.07 (t, J=5.5 Hz, 2H), 3.86-3.32 (m, 52H), 3.23-3.17 (m, 1H), 2.92 (dd, J=12.7, 5.0 Hz, 1H), 2.70 (d, J=12.7 Hz, 1H), 2.63-2.56 (m, 1H), 2.51-2.46 (m, 3H), 2.40-2.35 (m, 1H), 2.21 (t, J=7.4 Hz, 2H), 2.19-2.12 (m, 1H), 1.82-1.52 (m, 7H), 1.50-1.33 (m, 3H), 1.32-1.19 (m, 2H). MS (ESI): 1218 [M+H]⁺. HPLC: 100%, RT: 4.90 min.

RNA sequencing: Polyadenylated (poly-A) mRNA was isolated from 10 μg total RNA using Dynal oligo(dT) beads (Invitrogen). Poly-A mRNA was fragmented for five minutes at 70° C. using Fragmentation buffer from Ambion. First strand cDNA synthesis used random hexamer primers and SuperScriptII (Invitrogen). Second strand cDNA synthesis was performed using DNApolI (Invitrogen) and was purified using QIAquick PCR spin columns (Qiagen). Library preparation was performed according to manufacturer“s instructions (Illumina).

RNA-seq Alignment and Transcript Expression Analysis: 76-bp Illumina RNA-seq reads for a claudin-low tumor (3 lanes), SUM159 (4 lanes), and MDA-MB-231 (3 lanes) were obtained from the TCGA and aligned to the UCSC human knownGene mRNA from NCBI build 37 (hg19) using Bowtie. Langmead et al., 2009 Genome Biology 10, R25. The alignment was performed allowing just one mismatch in each read and only the best resulting alignment was reported for each aligned read. Duplicate reads were removed using Picard (http://picard.sourceforge.net) and in-house scripts were used to obtain read counts for protein kinases. Read counts were summed for all isoforms of each kinase gene. The raw kinase transcript read counts were then normalized with a calculation of reads per kilobase of exon model per million mapped reads (RPKM). Mortazavi et al., 2008 Nat Methods 5 621-628. The value of “N” (total number of mappable reads) in the RPKM formula was defined as the total number of aligned reads minus the duplicate reads. Additionally, the mean isoform length for each gene was used in the RPKM calculations. All read counts and RPKM values for each cell type are in Tables 1.

Western blotting: Proteins from cell lysates were separated by SDS-PAGE chromatography, transferred to nitrocellulose membranes, and probed with the indicated primary antibodies. Antibodies recognizing pAKT (S473), pAKT (T308), AURA, pAXL (Y702), AXL, c-Myc, DDR1, EGFR, pERK1/2 (T202/Y204), pHER3 (Y1197), MAX, pMEK1/2 (S217/S221), MEK1/2, MKP3, pP70S6K (T389), pPDGFRβ (Y751), pPDGFRβ (Y1009), pPDGFR (Y857), PDGFRβ, pRAF (S338), pRSK1 (T359/S363), pVEGFR2 (Y1175), VEGFR2 were obtained from Cell Signaling Technology. Antibodies for Cyclin A2, Cyclin B1, Cyclin D1, ERK2, RAF, and pRAF (S259) were obtained from Santa Cruz Biotechnology. The antibody recognizing Bim was obtained from Chemicon. The antibody recognizing p-c-Myc (S62) was obtained from Abeam. Secondary HRP-anti-rabbit and HRP-anti-goat secondary antibodies were from Jackson Immunoresearch Laboratories and Santa Cruz Biotechnology, respectively. Western blots were visualized by incubation with SuperSignal West Pico Chemiluminescent Substrate (Thermo Scientific).

Nuclear extract precipitation and immunoprecipitation: Nuclear extracts were isolated from SUM159 cells treated with DMSO or 5 μM AZD6244 for 4 and 72 hrs. Briefly, lysates were harvested in 200 μL of (10 mM HEPES (pH 7.9), 1.5 mM MgCl2, 10 mM KCl, 0.5% NP40, 1× protease inhibitor cocktail (Roche), and 1% each of phosphatase inhibitor cocktails 2 and 3 (Sigma)). Following 10 min on ice, cells were centrifuged for 15 min at 5, 000 rpm at 40 C. Nuclei fractions were collected by removing the supernatant and pellets were resuspended into 100 μL of (20 mM HEPES (pH 7.9), 25% Glycerol (v/v), 1.5 mM MgCl2, 0.5 mM EDTA, 0.5 M KCl, 1× protease inhibitor cocktail (Roche), and 1% each of phosphatase inhibitor cocktails 2 and 3 (Sigma)) and incubated on ice for 1 hrs. Following centrifugation at 14,000 rpm for 30 mM, supernatants were isolated and protein concentration determined by Bradford assay. For immunoprecipitations, cell lysates (300 μg protein) were immunoprecipitated with anti-MAX (Cell Signaling) and protein G-sepharose (Invitrogen) at 40 C overnight. The protein G-sepharose pellets were washed five times with cell lysis buffer (20 mM Tris (pH 7.5), 150 mM NaCl, 1 mM EDTA, 1 mM EGTA, 1% Triton X-100, 2.5 mM Sodium pyrophosphate, 1 mM β-glycerophosphate, 1 mM Na3VO4, 1 μg/ml Leupeptin) boiled in 40 μL of sample buffer for 5 min and resolved in SDS-PAGE. Transferred nitrocellulose membranes were then probed with anti-Myc (Cell Signaling) antibody to detect Myc-Max complexes.

RTK arrays: Cells were harvested in RTK array lysis buffer containing 20 mM Tris-HCl (pH 8.0), 1% NP-40, 10% glycerol, 137 mM NaCl, 2 mM EDTA, 1×EDTA-free protease inhibitor cocktail (Roche), and 1% each of phosphatase inhibitor cocktails 1 and 2 (Sigma). After incubating on ice for 20 minutes, cell debris was pelleted at 4° C. Lysates (500 μg protein) were applied to R&D Systems Proteome Profiler™ Human Phospho-RTK antibody arrays. Washing and secondary antibody steps were performed according to the manufacturer“s instructions. RTK arrays were visualized by SuperSignal West Pico Chemiluminescent Substrate (Thermo Scientific).

ChIP-PCR: Cells were fixed for 10 min in 1% formaldehyde, sonicated (VCX130 Ultrasonicator), and immunoprecipitated with 5 μg anti-c-Myc and protein A dynabeads (Invitrogen). Crosslinking was reversed by overnight incubation at 65° C., and DNA was purified with the MinElute PCR purification kit (QIAGEN). ChIP assay was quantified by real-time PCR using Absolute Blue SYBR green PCR mix (Thermoscientific). Fold enrichment was determined by the 2̂-ΔCT method using the following PCR primers designed to amplify 75-100 bp fragments from genomic DNA: (SEQ ID NOs. 1-4) forward 5′-GGCTTTGAGACGTGAAAAGGA-3′ and reverse 5′-GGTCATCCAGCACAGATTGGA-3′; forward 5′-TGGGCCTTGGTTTGTCCTT-3′ and reverse 5′-CATGGAGGAGATGGAAAGATCCT-3′.

### 6.17. Inhibitor

### Bead Coupling Procedures

### 6.17.1. Carbodiimide Coupling

Each kinase inhibitor is re-suspended in 50:50 dimethylformamide (DMF):ethanol (v/v) to give a final concentration of ˜16 mM. An equal volume of ECH (or EAH) sepharose 4B beads (GE Healthcare), pre-washed in 50:50 DMF:ethanol, is then added to the inhibitor solution, along with the carbodiimide EDC (Sigma) to a final concentration of 0.2M. The drug-bead suspension is incubated overnight at 4° C., followed by a two-hour room-temperature incubation in 1 M Ethanolamine and 0.2 M EDC (in 50:50 DMF:ethanol). The coupled beads are then washed alternately with acidic and basic buffers and re-suspended in 20% ethanol for storage.

Inhibitor-conjugated bead preparation: Inhibitor beads were prepared via carbodiimide coupling of kinase inhibitors to ECH Sepharose 4B (Lapatinib, Bisindoylmaleimide-X, SB203580, Dasatinib, PP58 and VI16832) or EAH Sepharose 4B (Purvalanol B) (GE Healthcare). Briefly, ECH-Sepharose and EAH-Sepharose beads were washed with 50% DMF/EtOH followed by incubation with kinase inhibitors in 50% DMF/EtOH and 0.1M EDC (Sigma) at pH 5-6 overnight at 40 C in the dark. Following coupling, excess remaining groups were blocked with 0.1M N-ethyl-N″-(3-dimethylaminopropyl) carbodiimide hydrochloride (EDC) in 50% DMF/EtOH 1M ethanolamine (ECH-Sepharose) or 20 mM HAc in 50% DMF/EtOH (EAH-Sepharose). Subsequently, beads were washed with 50% DMF/EtOH and alternating washes of 0.1 M Tris-HCl (pH 8.3) and 0.1 M acetate (pH 4.0) buffers, each containing 0.5 M NaCl. Inhibitor beads were stored in 20% ethanol at 4° C. in the dark.

### 6.17.2. Biotin-Strepavidin Coupling

Each biotinylated kinase inhibitor is re-suspended in the binding buffer, phosphate-buffered saline (0.1M phosphate, 0.15M NaCl; pH 7.2) at final concentration of 5-15 mM. An equal volume of High Capacity Streptavidin Agarose beads (Pierce), pre-washed in Binding/Wash Buffer (Pierce), is then added to the inhibitor solution and incubated for 30 mM or overnight at 4° C. The drug-bead suspension is then washed with 10 column volumes of Binding Buffer (Pierce) and re-suspended in 20% ethanol for storage.

For kinome affinity purification, cells are lysed on ice for 20 minutes in lysis buffer containing 50 mM HEPES (pH 7.5), 0.5% Triton X-100, 150 mM NaCl, 1 mM EDTA, 1 mM EGTA, 10 mM sodium fluoride, 2.5 mM sodium orthovanadate, 1× protease inhibitor cocktail (Roche), and 1% each of phosphatase inhibitor cocktails 2 and 3 (Sigma). Cell lysate are sonicated (3×10 s) on ice and centrifuged for 15 min (13,000 rpm) at 4° C. and the supernatant was collected and syringe-filtered through a 0.2 uM SFCA membrane. The filtered lysate (approximately 20-40 mg of protein per experiment) was brought to 1M NaCl and pre-cleared by flowing over 500 ul of blocked and washed NHS-activated Sepharose 4 Fast Flow beads (GE Healthcare). The flow-through was collected and passed through a column of layered biotinylated-inhibitor-conjugated beads (Bisindoylmaleimide-X (50 ul), SB203580 (50 ul), Lapatinib (100 ul), Dasatinib (100 ul), Purvalanol B (100 ul), VI16832 (100 ul), PP58 (100 ul)) to isolate protein kinases from the lysates. Kinase-bound inhibitor beads were washed with 20 ml of high-salt buffer and 10 ml of low-salt buffer, each containing 50 mM HEPES (pH 7.5), 0.5% Triton X-100, 1 mM EDTA, 1 mM EGTA, and 10 mM sodium fluoride, and 1M NaCl or 150 mM NaCl, respectively. A final wash of 1 ml 0.1% SDS was applied to the columns before elution in 1 ml of a 0.5% SDS solution in high heat. Elutions from all columns were combined and cysteines were alkylated by sequential incubations with DTT (final concentration 5 mM) for 20 min at 60° C. and iodoacetamide (final concentration 20 mM) for 30 min at room temperature in the dark. The elution was spin-concentrated to 100 ul and detergents were removed by a chloroform/methanol extraction. Briefly, 400 ul of HPLC-grade methanol, 100 ul HPLC-grade chloroform, and 300 ul HPLC-grade water was added to the 100 ul concentrated elution, with vortexing and centrifugation at 13,000 rpm between each addition. After a final mixing, the sample was centrifuged for 5 min to pellet the protein at the interface and the upper phase was removed with care to leave the protein pellet intact. The protein pellet and lower phase were resuspended in 300 ul of methanol, and the sample was again vortexed and centrifuged for 5 min to pellet the protein at the bottom of the tube. The supernatant was removed and one or more methanol washes were performed to ensure the removal of detergents.

### 6.18. Multiplexed Inhibitor Bead Affinity Chromatography

Cells were lysed on ice for 20 minutes in lysis buffer containing 50 mM HEPES (pH 7.5), 0.5% Triton X-100, 150 mM NaCl, 1 mM EDTA, 1 mM EGTA, 10 mM sodium fluoride, 2.5 mM sodium orthovanadate, 50 ng/mL calyculin A, 1× protease inhibitor cocktail (Roche), and 1% each of phosphatase inhibitor cocktails 2 and 3 (Sigma). Cell lysate was sonicated (3×10 s) on ice and centrifuged for 15 minutes (13,000 rpm) at 4° C. and the supernatant was collected and syringe-filtered through a 0.2 μM SFCA membrane. The filtered lysate (approximately 40 mg or protein per experiment) was brought to 1 M NaCl and pre-cleared by flowing over 500 μL of blocked and washed NHS-activated Sepharose 4 Fast Flow beads (GE Healthcare). The flow-through was collected and passed through a column of layered inhibitor-conjugated beads (Bisindoylmaleimide-X (50 μL), SB203580 (50 Lapatinib (100 μL), Dasatinib (100 μL), Purvalanol B (100 μL), VI16832 (100 μL), PP58 (100 4)) to isolate protein kinases from the lysates. Kinase-bound inhibitor beads were washed with 20 mL of high-salt buffer and 10 mL of low-salt buffer, each containing 50 mM HEPES (pH 7.5), 0.5% Triton X-100, 1 mM EDTA, 1 mM EGTA, and 10 mM sodium fluoride, and 1 M NaCl or 150 mM NaCl, respectively. A final wash of 1 mL 0.1% SDS was applied to the columns before elution in 1 mL of a 0.5% SDS solution in high heat. Elutions from all columns were combined and cysteines were alkylated by sequential incubations with DTT (final concentration 5 mM) for 20 minutes at 60° C. and iodoacetamide (final concentration 20 mM) for 30 minutes at room temperature in the dark. The elution was spin-concentrated to 100 μL and detergents were removed by a chloroform/methanol extraction.

Briefly, 400 μL of HPLC-grade methanol, 100 μL HPLC-grade chloroform, and 300 μL HPLC-grade water was added to the 100 μL concentrated elution, with vortexing and centrifugation at 13,000 rpm between each addition. The protein in the sample precipitates upon the addition of water, and localizes to the interface that forms. After a final mixing, the sample was centrifuged for 5 minutes to pellet the protein at the interface and the upper phase was removed with care to leave the protein pellet intact. The protein pellet and lower phase were resuspended in 300 μL of methanol, and the sample was again vortexed and centrifuged for 5 minutes to pellet the protein at the bottom of the tube. The supernatant was removed and at least one more methanol wash was performed to ensure the full removal of detergents.

### 6.19. MIB Statistical Analysis

Data obtained from the MALDI TOF/TOF was processed with the ProteinPilot software (v 3.0) to identify proteins from database searches and quantitate changes in binding of kinases to MIBs using the Paragon Algorithm. The search results are further processed by the Pro Group Algorithm to determine the smallest justifiable set of detectable proteins and relative protein levels. Three replicates of SILAC labeled SUM159 cells treated with AZD6244 (2 ‘heavy’, 1 ‘light’) or DMSO were performed to assess the reproducibility of MIB kinase affinity capture. A total of 113 unique kinases are identified. For each kinase, the pool protein ratio and p-value across the three replicates was calculated as follows.

Let yᵢⱼ denote the log 2 protein ratio for of kinase i, i=1, . . . , 113 in replicate j, j=1, 2, 3. The pool protein ratio for kinase i is defined as 2^{yi}, where

$${y}_{i} = \frac{{\sum }_{j = 1}^{3} {y}_{ij}}{3} .$$

To avoid directional conflict, the two-sided p-value reported in ProteinPilot was converted to one-sided p-value and denote it as pᵢⱼ. Stouffer's z-score method was applied to combine the p-values. Let zᵢⱼ=Φ⁻¹(1−pᵢⱼ), where Φ is the standard Gaussian cumulative distribution function. Define the combined Z-score as

$${z}_{i} = \frac{{\sum }_{j = 1}^{3} {z}_{ij}}{\sqrt{3}} .$$

The combined two-sided p-value for kinase i is given as pᵢ=2(1−Φ(|zᵢ|)).

FIG. 15A shows the pairwise plot of the log 2 protein ratio (FIG. 15B, negative log p-values) for the replicates and the pooled data. The Pearson correlation coefficients printed in the upper corner indicate high reproducibility of the MIB kinase affinity capture technique.

Next, the overlap/concordance between the kinases ranked by p-values was assessed for any two pairs of replicates. FIG. 16 below shows that the overlap/concordance between the individual replicates is greater than 70% among the top 20 kinases. In addition, the overlap is identify kinases which exhibit statistically significant changes in expression based on Benjamini-Hochberg adjusted p-values at FDR of 0.05 to account for multiple comparisons. 24, 13 and 10 kinases are identified to be statistically significant for replicate 1, 2 and 3, respectively. FIG. 17 displays the Venn diagram of the overlaps between these lists of significant kinases. On the other hand, 24 kinases are identified to be statistically significant in the pooled p-values. The list of significant kinases is given in Table 2.

TABLE 2
List of kinases which are significant at FDR of 0.05. “Union Rep1, Rep2,
Rep3” is the list of unique kinases obtained by merging the three lists of
significant kinases from Row1, Row2 and Row3.

| | No. of significant | |
|---|---|---|
| Sample | kinases | List of significant kinases |
| Rep1 | 24 | “AAK1” “ACK1” “ACVR1” “CDC2” “CDK5” |
| | | “CDK6” “CSK” “CSK22” “EPHA2” “GAK” “KC1D” |
| | | “KCC2D” “KCC2G” “KPCD1” “KPCD3” “ERK2” |
| | | “MEK2” “NEK9” “PDGFRB” “PMYT1” “PRKDC” |
| | | “MST1” “TTK” “AXL” |
| Rep2 | 13 | “ACVR1” “CDK5” “CDK6” “CSK22” “EGFR” |
| | | “EPHA2” “KS6A3” “MEK2” “PDGFRB” “PMYT1” |
| | | “PRKDC” “RIOK2” “TBK1” |
| Rep3 | 10 | “CDC2” “CDK6” “DDR1” “EPHA2” “EPHB4” |
| | | “MEK2” “PDGFRB” “PMYT1” “MST1” “AURA” |
| Pooled | 24 | “ACVR1” “CDC2” “CDK5” “CDK6” “CSK22” |
| | | “DDR1” “EGFR”“EPHA2” “FAK2” “KCC2D” |
| | | “KCC2G” “KPCD3” “M3K11” “ERK1” “MEK1” |
| | | “MEK2”“PDGFRB” “PMYT1” “PRKDC” “RIPK2” |
| | | “MST1” “AURA” “TTK” “AXL” |
| Union (Rep1, | 31 | “AAK1” “ACK1” “ACVR1” “CDC2” “CDK5” |
| Rep2, Rep3 list) | | “CDK6” “CSK”“CSK22” “DDR1” “EGFR” “EPHA2” |
| | | “EPHB4” “GAK” “KC1D”“KCC2D” “KCC2G” |
| | | “KPCD1” “KPCD3” “RSK2” “ERK2” |
| | | “MEK2”“NEK9” “PDGFRB” “PMYT1” “PRKDC” |
| | | “RIOK2” “MST1”“AURA” “TBK1” “TTK” “AXL” |

### 6.20. Cell Viability Assays Using siRNA Knock Down of Protein Kinases

siGENOME pooled siRNAs for the genes of interest were obtained from Dharmacon, Thermo Scientific. RNAi assays were performed in either 96- or 384-well clear bottom plates. Prior to the assay, transfection conditions were optimized for SUM159 or MDA-MB-231 cells using Dharmafect transfection reagent and siRNAs for GAPDH (negative control), and UBB (lethality control). A 40 μl mixture of Dharmafect and siRNA was plated into each well by a multi-channel pipette and then followed by adding 160 μl cell suspension using a microplate dispenser. The final assay volume was 200 μl with a dose of 25 nM siRNA. Drug or vehicle solvent was added to the cell suspension before plating the cells. The assay was performed in triplicate and each plate had quadruple positive (UBB) and negative (GAPDH) controls. After 96 h incubation at 37° C. with 5% CO2, the number of viable cells in each well was determined by a luminescence viability assay with a Pherastar microplate reader. The % activity was calculated against the averages of positive and negative controls (% activity=100×(1−[raw value−σp]/[σn−σp], where σp and σn are averages of raw values for the positive and negative controls, respectively. Each median in triplicate was used as a representative of % activity in the figures. Two-to-three independent experiments were performed with each cell line and siRNA.

### 6.21. Deconvolution of siRNA Pools

Individual siRNAs from SMARTpools were tested for ability to knockdown target proteins. Two of four of the individual siRNAs showing target knockdown was required for acceptance of the SMARTpool phenotype. In addition, stable shRNA knockdown of PDGFRβ was shown in both SUM159 and MDA-MB-231 cells to have a synthetic lethal-like phenotype when cells were treated with AZD6244 or U0126.

### 6.22. Phosphoproteomics Analysis of MIBs

Phosphopeptides were enriched from MIB elution digests using TiO2 beads as previously described (Thingholm et al., 2006 Nat Protocola 1, 1929-1935. Tryptic peptides were separated by reverse phase nano-HPLC using a nanoAquity UPLC system (Waters Inc). Peptides were first trapped in a 2 cm trapping column (75 μm ID, C18 beads of 2.5 μm particle size, 200 Å pore size) and then separated on a self-packed 25 cm column (75 μm ID, C18 beads of 2.5 μm particle size, 100 Å pore size) at room temperature. The identity and phosphorylation status of the eluted peptides was determined with a Velos-Orbitrap mass spectrometer (Thermo-Scientific). Specifically, following a FT full scan, MS2 spectral data were acquired by one of three dissociative methods on the 9 most intense ions from the full scan, taking into account dynamic exclusions. For ion dissociation, collision induced dissociation (CID), high energy collision induced dissociation (HCD) or a CID/HCD toggle was employed. The polysiloxane lock mass of 445.120030 was used throughout. All raw data were converted to mzXML format and then searched using Sequest on a Sorcerer 2.0 platform (Sage N Research, Milpitas, Calif.). The search was semi-tryptic on the human IPI database (Oct. 3, 2010) appended with reversed sequences as decoys. Dynamic modifications for phosphorylated serines, threonines, and tyrosines were used, as well as a static modification for carbamidomethylated cysteines. Another search was also performed with the SpectraST algorithm provided in the Transproteomic Pipeline (TPP) version 4.4.1 using the NIST human ion trap database (Jan. 14, 2010). Results from the Sequest and SpectraST searches were analyzed using TPP's PeptideProphet and then combined using IProphet (Shteynberg et al., 2011 Mol Cell Proteomics 10 M111.007690 1-15). SILAC ratios were calculated with the XPRESS algorithm within TPP.XPRESS parameters were heavy arginines' with a mass difference of 10 and heavy lysines' with a mass difference of 6. Protein identifications were output by TPP's ProteinProphet.

### 6.23. Immunofluorescence and TUNEL Assays

Tumors were snap frozen, cryosectioned at 6 μm and fixed in 4% paraformaldehyde for 15 min. Sections mounted on glass slides were incubated overnight with PDGFRβ rabbit antibody (Cell Signaling #3169) at 1:1000 dilution. Secondary antibody was Alexa 555 goat-anti rabbit. Protocol provided by Cell Signaling for staining of cryosections was followed. TUNEL assays were performed using the In Situ Death Detection Kit per manufacturers protocol (Roche, #12156792).

### 6.24. Kinases from Additional Diseases/Tissues

Using the methods described above, the kinome for a head and neck cancer cell line was studied before and after treatment. The cell line was HN12 head and neck cancer cell line. Cells were cultured using standard methods in 150 mL culture flasks and were treated with 100 nM Rapamycin for 1 hr and 20 mg of protein run on the standard MIB cocktail outline using the column procedure in Section 6.18 above and the mass spectroscopy methods in Section 6.22. FIG. 18 shows the changes in the kinome before and after treatment with rapamycin in DMSO.

Using the methods described above, the kinome for a leukemia cell line was studied before and after treatment with a MERTK inhibitor. 697 leukemia cells were treated with 50 nM of the MERTK inhibitor of interest for 2 hrs and treated with pervanadate 10 min before harvesting. 20 mg of protein were run over the standard MIB cocktail outlined above; see the chromatography in Section 6.18 and the mass spectroscopy methods in Section 6.22. FIG. 19 shows the changes in the kinome for a leukemia cell line before and after treatment with MERTK inhibitor in DMSO.

Using the methods described above, the kinome for cells pre- and post-CMV infection. Specifically, primary human foreskin fibroblasts were infected with CMV (strain AD169) for 72 hrs and 7 mg of protein run on the standard MIB cocktail and mass spectroscopy analysis; see the chromatography in Section 6.18 and the mass spectroscopy methods in Section 6.22. FIG. 20 shows the changes in the kinome for the fibroblast cells before and after CMV infection.

### 6.25. Pancreatic Cancer Preliminary Results

The successive disappointments of new therapies for pancreatic cancer coupled with the critical need to rapidly translate findings to the clinic have prompted a systems based approach to define the activated kinome in primary and metastatic pancreatic cancer. For pancreatic cancer where kinase mutations are uncommon, determination of kinase activation profiles will be key to tailoring the use of kinase inhibitors for effective therapy. Several resources are leveraged simultaneously for successful kinome profiling of pancreatic cancer, including the novel MIB/MS approach described above, a library of snap frozen pancreatic tumors from, an expanding library of more than 38 different patient-derived xenografts (PDX) of pancreatic cancer, and an active clinical trial using BKM120, a pan-class 1 phosphatidylinositol 3-kinase (PI3K) inhibitor. This is a Phase I/II clinical trial with a planned Phase II expansion cohort in pancreatic cancer. Phase I began accrual May 2012.

The methods described herein and the ability to measure a wide spectrum of the kinome by quantitative proteomics in cell lines and tumors alike is highly unique. A major advance in using this chemical proteomic technology is the activity-dependent measurement of the “untargeted and understudied” kinome. There simply are no reagents for many of these kinases thereby limiting understanding of their regulation. MIB/MS overcomes these limitations and detects changes in kinases for which reagents such as anti-phospho-antibodies cannot distinguish closely related kinases (e.g. different activity states of MEK1 vs MEK2, ERK1 vs ERK2). In addition MIB/MS easily distinguishes the regulation of related receptor tyrosine kinases (RTKs), e.g., DDR1 and DDR2, for which limited antibodies are available.

Applying these methods to interrogate the kinome of tumors originally collected from pancreatic cancer patients eliminates the traditional challenges and time necessary to translate the relevancy of research from two dimensional cell lines to patients. This approach inherently precludes the need to know the mechanism, genetic or otherwise, underlying kinase activation, and instead attempts to focus first on what kinases are activated. Determining the intrinsic state of kinome activation is critical to narrowing the focus of the studies on specific kinase inhibitors for tumor subtypes, both primary and metastatic tumors. In addition the simultaneous quantitative assessment of kinome activation using MIB/MS allows rational determination of combinatorial approaches for therapies. This reverse approach determines a priori which of 150-320 kinases to focus on and using the well-established preclinical models to quickly extend further studies of key kinases in order to validate and design new therapeutic strategies that can be applied to the clinic.

There is growing evidence that the approach of targeting multiple pathways simultaneously will be the future of personalized therapies. As combinatorial therapies evolve, there is a critical need to identify driver alterations a priori to tailor treatment. For example, studies suggest that PI3K pathway activation is associated with resistance to MEK inhibitor treatment. In fact, ongoing Phase I/II clinical trials of combination therapy with PI3K and MEK inhibitors have shown early promise in patients with advanced solid tumors (Bendell et al., AACR 102nd Annual Meeting, 2011). Previous studies using MIB/MS profiling of the kinome response to MEK inhibitor uncovered a novel and surprising mechanism for drug resistance in preclinical models of TNBC. Using a faithful genetically engineered mouse model (GEMM) of TNBC, the studies above showed it was possible to restore sensitivity to the MEK inhibitor by inhibiting RTKs activated in response to MEK inhibitor, thus eliciting tumor regression. “Kinomining” using MIB/MS identified the profile of activated RTKs that lead to a rational prediction of a combination therapy that was effective in the C3-Tag TNBC GEMM.

LCCC1036 is a Phase I study of BKM120 in combination with a modified (m) FOLFOX (5-fluorouracil, leucovorin, and oxaliplatin; mFOLFOX6) in patients with advanced solid tumors, with a planned expansion cohort in patients with metastatic pancreatic cancer. As multiple PI3K inhibitors have shown preclinical promise in pancreatic cancer, trials are underway to assess their effectiveness in patients. The MIB/MS approach is distinctive in a number of ways include the fact that it accurately assays small samples such as core biopsies from patients.

The kinome response was studied for pancreatic cancer cell lines and PDX tumors to PI3K inhibitors. Similar to the studies above for breast cancer where kinome reprogramming occurred through the expression and activation of RTKs in response to MEK inhibition, pancreatic cancer cell lines and tumors also alter their kinome activation profile in response to kinase inhibitor treatment. It is important to evaluate whether kinome reprogramming can be demonstrated in patients undergoing BKM120 therapy. The rationale for the inclusion of this particular clinical trial is multifactorial. First, the analysis of metastatic tumors from patients will provide a unique validation opportunity for the studies of kinome activation in an existing library of pancreatic tumors. Second, studies in preclinical models will directly parallel this trial, so as to quickly focus on a manageable number of candidate kinases that may confer drug resistance. Third, regardless of whether BKM120 is clearly effective in patients, by leveraging the clinical trial and preclinical studies simultaneously, exactly which combinatorial strategy should be used for BKM120 may be determined. Finally, the additive information from pretreated patients in LCCC1036 will identify kinase inhibitors to secure for a follow-up clinical trial or trials.

Kinase Gene Expression Identifies Candidate Subtypes: Whole exome sequencing of pancreatic cancer has shown significant heterogeneity within primary tumors and between metastases. A gene expression dataset of 70 primary and 6 metastatic tumors was analyzed using the Consensus ClusterPlus (CCP) algorithm to determine if there was significant heterogeneity in protein kinase expression at the transcript level. The tumor kinase profiles could be categorized into 4 subtypes. Based on functional pathway enrichment analysis using Ingenuity Pathway Analysis (IPA, Ingenuity), the 4 subtypes are (1) RTK, (2) migration (3) survival and (4) proliferation. Characterization of the kinome subtypes is being further interrogated by RNAseq that will give even greater depth and quantitative measure of protein kinase expression in the tumors.

MIB/MS Technology Reproducibility: The high reproducibility of the MIB kinase affinity capture technique was described above. Triplicate experiments showed large pairwise Pearson correlation coefficients among the log 2 kinase ratios (r²≈0.6), negative log p-values (r²≈0.55) and number of peptides (r²≈0.95). The overlap/concordance between the individual MIB/MS replicates was greater than 70% among the top 20 kinases.

Kinase gene expression defines 4 possible subtypes: A list of kinases was derived from the Gene Ontology database (http://www.genenames.org). The gene expression of kinases was extracted from a microarray dataset of human primary and metastatic tumors and used to determine whether kinomic subtypes of pancreatic cancer could be defined by ConsensusClusterPlus.

Kinome landscape and kinomic subtypes in pancreatic cancer: The studies with breast cancer have shown that the activation state of the kinome and the reprogramming response to specific kinase inhibitors (e.g., lapatinib, AZD6244, sorafenib) is unique for each subtype (luminalA/B, Her2+, TNBC basal and claudin-low). MIB/MS profiling effectively and reproducibly defined the kinome signature of tumors in terms of activation of kinases and changes in kinome activation in response to drug. Thus, MIB/MS profiling has the potential for the first time to define patterns of kinome activation and response to drug that would identify functional pancreatic tumor kinome subtypes. Using the MIB/MS technology, an initial set of 12 primary pancreatic PDX tumors was profiled. Using CCP on this initial set of primary tumors, three potential kinase subtypes of primary pancreatic cancer were found. Using functional enrichment analysis (IPA, Ingenuity) the three subtypes contained kinases enriched for functions of proliferation, survival, and migration. The dynamic range of kinase activation even in this small set of samples suggests that the classification will become more distinct as the sample size increases.

Kinome reprogramming in pancreatic cancer—cell type and inhibitor specific. The kinome response to drug in 3 pancreatic cancer cell lines (HPAC, AsPC-1, HPAF-II) was also evaluated using 3 kinase inhibitors that are currently in Phase I/II clinical trials: BKM120 (Novartis), an oral pan-class 1 PI3K inhibitor, GSK2126258 (Glaxo Smith-Kline), an ATP competitive PI3K/mTOR inhibitor, and GSK1120212, a reversible allosteric inhibitor of MEK1/2 (Glaxo Smith-Kline). The kinome response for GSK2126458 and GSK1120212 was compared using MIB/MS and RTK arrays to determine if kinome reprogramming was inhibitor-specific. In HPAC cells GSK2126458 inhibited p70 S6 kinase activity consistent with mTOR inhibition while GSK1120212 inhibited MEK1 activation. GSK2126458 induced ALK, RET, INSR and IGF-1R phosphorylation whereas treatment with either BKM120 or GSK1120212 did not. MIB/MS profiling also showed that GSK2126258 altered the kinome profile differently from GSK1120212. Thus, kinome reprogramming is target-specific.

Despite harboring common driver mutations in KRAS, TP53, and CDKN2, baseline RTK phosphorylation varied across the 3 cell lines. In addition, the RTK phosphorylation profile in response to treatment with the 3 inhibitors varies. Thus, intrinsic kinome profiles and kinome reprogramming is cell line-specific.

PDX Program: Mouse models of primary human tumors are increasingly recognized to provide a significant advancement over conventional cell line xenograft models. Several patient derived xenograft models (PDX) were established successfully engrafting 44 different pancreatic ductal adenocarcinoma (PDAC) PDX. Recent reports in lung, pancreatic, and colon cancers, as well as glioblastomas (GBM), suggest that patient tumors directly explanted in immunecompromised mice exhibit response rates to cytotoxic or targeted therapies in keeping with what is observed in. Hidalgo 2011; DeRose 2011. Unlike established cell lines where there is dependence on subpopulations that are able to survive on a plastic dish, these tumors retain the heterogeneity of the original tumors and their original histological characteristics.

Kinome reprogramming in PDX model of pancreatic cancer after BKM120 treatment: The kinome response of a pancreatic cancer PDX to BKM120 treatment was evaluated using MIB/MS. BKM120 inhibited AKT phosphorylation consistent with PI3K inhibition.

Kinome reprogramming in pancreatic cancer cell lines: HPAC, AsPC-1, HPAF-II cell lines were treated with either GSK1120212, GSK2126458, BKM120 or vehicle control and harvested at 48 h. Normalized spot intensity of RTK arrays were measured and activated and repressed RTK were determined in response to either GSK1120212 or GSK2126458 4 h and 72 h after treatment. Treatment with BKM120 resulted in both expected and unexpected changes in the kinome. Although the sample size was small, BKM120 was not clearly effective in the PDX model. Together this data suggests that upstream signaling pathways that were activated in response to BKM120 may be associated with BKM120 resistance and should be investigated. These data will help determine combinatorial strategies for BKM120 therapy.

Kinome reprogramming in PDX models: De-identified tumors from patients are grafted into immunecompromised mice and passaged. All tumors are characterized using microarray and mutational analysis. Hematoxylin & eosin staining of a patient tumor at the time of operation and after several passages over a 1.5 yr period showing similar glandular architecture with surrounding stroma. Variable levels of pERK1/2 and pAKT(S473) was seen across pancreatic cancer PDX. PDX P100422 with moderate levels of pAKT was selected for BKM120 treatment. pAKT expression in BKM120 versus vehicle treated PDX tumor. Mice were treated daily with either vehicle control (n=3) or BKM120 (n=3) and tumors were harvested 4-6 h after the last dose. PDX tumors were treated daily for 28 days. 4 tumors in the BKM120 growth showed tumor growth inhibition relative to controls. The 3 vehicle control and 3 BKM120 treated tumors showing inhibition of pAKT were analyzed using MIB/MS. Activation and repression of the kinome in response to PI3K inhibition in PDX P100422.

Window trial in TNBC using MEK inhibitor GSK1120212 shows patient tumor kinome reprogramming. Kinome reprogramming in TNBC patients treated with the MEK inhibitor GSK1120212 is being evaluated (LCCC 1122). In this window trial, pre-treatment core needle biopsies are compared to similar amounts of 7-day post-treatment tumors procured at the time of operation. Preliminary results from this trial show two important points. First, this demonstrates the ability to use MIB/MS profiling with small protein amounts obtained from core biopsies and provides proof-of-concept for patient pancreatic tumor studies using MIB/MS. Second, the results show that GSK1120121 effectively inhibited the MEK-ERK pathway and resulted in kinome reprogramming with the upregulation of MRCKG, FER and DDR1

TABLE 3

| Family | Patient TNBC | SUM159 | MDA231 | C3Tag |
|---|---|---|---|---|
| Ser/Thr | AAK1 | AAK1 | AAK1 | AAK1 |
| CAMK | AAPK1 | AAPK1 | AAPK1 | AAPK1 |
| CAMK | AAPK2 | AAPK2 | AAPK2 | AAPK2 |
| TK | ABL1 | ABL1 | ABL1 | ABL1 |
| TK | ABL2 | ABL2 | ABL2 | ABL2 |
| TK | ACK1 | ACK1 | ACK1 | ACK1 |
| TKL | ACV1B | ACV1B | ACV1B | ACV1B |
| TKL | ACVR1 | ACVR1 | ACVR1 | ACVR1 |
| Atypical | ADCK3 | ADCK3 | ADCK3 | ADCK3 |
| Atypical | ADCK4 | ADCK4 | ADCK4 | ADCK4 |
| TK | ALK | ALK | ALK | ALK |
| TKL | ARAF | ARAF | ARAF | ARAF |
| Atypical | ATM | ATM | ATM | ATM |
| Atypical | ATR | ATR | ATR | ATR |
| AGC | AURKB | AURKB | AURKB | AURKB |
| AGC | AURKC | AURKC | AURKC | AURKC |
| TKL | AVR2A | AVR2A | AVR2A | AVR2A |
| TK | AXL | AXL | AXL | AXL |
| TK | BLK | BLK | BLK | BLK |
| TKL | BMP2K | BMP2K | BMP2K | BMP2K |
| TKL | BMPR2 | BMPR2 | BMPR2 | BMPR2 |
| TKL | BMR1A | BMR1A | BMR1A | BMR1A |
| TKL | BMR1B | BMR1B | BMR1B | BMR1B |
| TKL | BRAF1 | BRAF1 | BRAF1 | BRAF1 |
| Atypical | BRD3 | BRD3 | BRD3 | BRD3 |
| Atypical | BRD4 | BRD4 | BRD4 | BRD4 |
| TK | BTK | BTK | BTK | BTK |
| Ser/Thr | BUB1 | BUB1 | BUB1 | BUB1 |
| Ser/Thr | CCRK | CCRK | CCRK | CCRK |
| CMGC | CD2L5 | CD2L5 | CD2L5 | CD2L5 |
| CMGC | CD2L6 | CD2L6 | CD2L6 | CD2L6 |
| CMGC | CD2L7 | CD2L7 | CD2L7 | CD2L7 |
| CMGC | CDC2 | CDC2 | CDC2 | CDC2 |
| CMGC | CDK10 | CDK10 | CDK10 | CDK10 |
| CMGC | CDK2 | CDK2 | CDK2 | CDK2 |
| CMGC | CDK3 | CDK3 | CDK3 | CDK3 |
| CMGC | CDK4 | CDK4 | CDK4 | CDK4 |
| CMGC | CDK5 | CDK5 | CDK5 | CDK5 |
| CMGC | CDK6 | CDK6 | CDK6 | CDK6 |
| CMGC | CDK7 | CDK7 | CDK7 | CDK7 |
| CMGC | CDK8 | CDK8 | CDK8 | CDK8 |
| CMGC | CDK9 | CDK9 | CDK9 | CDK9 |
| CMGC | CDKL5 | CDKL5 | CDKL5 | CDKL5 |
| Metabolic | CHKA | CHKA | CHKA | CHKA |
| Metabolic | CHKB | CHKB | CHKB | CHKB |
| CMGC | CLK1 | CLK1 | CLK1 | CLK1 |
| CMGC | CLK2 | CLK2 | CLK2 | CLK2 |
| CMGC | CLK3 | CLK3 | CLK3 | CLK3 |
| CMGC | CLK4 | CLK4 | CLK4 | CLK4 |
| TK | CSF1R | CSF1R | CSF1R | CSF1R |
| TK | CSK | CSK | CSK | CSK |
| Ser/Thr | CSK21 | CSK21 | CSK21 | CSK21 |
| Ser/Thr | CSK22 | CSK22 | CSK22 | CSK22 |
| AGC | CTRO | CTRO | CTRO | CTRO |
| CAMK | DAPK1 | DAPK1 | DAPK1 | DAPK1 |
| Metabolic | DCK | DCK | DCK | DCK |
| TK | DDR1 | DDR1 | DDR1 | DDR1 |
| TK | DDR2 | DDR2 | DDR2 | DDR2 |
| CMGC | DYR1A | DYR1A | DYR1A | DYR1A |
| Ser/Thr | E2AK1 | E2AK1 | E2AK1 | E2AK1 |
| Ser/Thr | E2AK2 | E2AK2 | E2AK2 | E2AK2 |
| Ser/Thr | E2AK4 | E2AK4 | E2AK4 | E2AK4 |
| TK | EGFR | EGFR | EGFR | EGFR |
| TK | EPHA1 | EPHA1 | EPHA1 | EPHA1 |
| TK | EPHA2 | EPHA2 | EPHA2 | EPHA2 |
| TK | EPHA3 | EPHA3 | EPHA3 | EPHA3 |
| TK | EPHA4 | EPHA4 | EPHA4 | EPHA4 |
| TK | EPHA5 | EPHA5 | EPHA5 | EPHA5 |
| TK | EPHA6 | EPHA6 | EPHA6 | EPHA6 |
| TK | EPHA7 | EPHA7 | EPHA7 | EPHA7 |
| TK | EPHA8 | EPHA8 | EPHA8 | EPHA8 |
| TK | EPHAA | EPHAA | EPHAA | EPHAA |
| TK | EPHB1 | EPHB1 | EPHB1 | EPHB1 |
| TK | EPHB2 | EPHB2 | EPHB2 | EPHB2 |
| TK | EPHB3 | EPHB3 | EPHB3 | EPHB3 |
| TK | EPHB4 | EPHB4 | EPHB4 | EPHB4 |
| TK | EPHB6 | EPHB6 | EPHB6 | EPHB6 |
| TK | ERBB2 | ERBB2 | ERBB2 | ERBB2 |
| Ser/Thr | ERN1 | ERN1 | ERN1 | ERN1 |
| TK | FAK1 | FAK1 | FAK1 | FAK1 |
| TK | FAK2 | FAK2 | FAK2 | FAK2 |
| TK | FER | FER | FER | FER |
| TK | FES | FES | FES | FES |
| TK | FGFR1 | FGFR1 | FGFR1 | FGFR1 |
| TK | FGFR2 | FGFR2 | FGFR2 | FGFR2 |
| TK | FGFR3 | FGFR3 | FGFR3 | FGFR3 |
| TK | FGFR4 | FGFR4 | FGFR4 | FGFR4 |
| TK | FGR | FGR | FGR | FGR |
| Atypical | FRAP | FRAP | FRAP | FRAP |
| TK | FRK | FRK | FRK | FRK |
| TK | FYN | FYN | FYN | FYN |
| Ser/Thr | GAK | GAK | GAK | GAK |
| CMGC | GSK3A | GSK3A | GSK3A | GSK3A |
| CMGC | GSK3B | GSK3B | GSK3B | GSK3B |
| Ser/Thr | GUC2D | GUC2D | GUC2D | GUC2D |
| TK | HCK | HCK | HCK | HCK |
| CMGC | HIPK1 | HIPK1 | HIPK1 | HIPK1 |
| CAMK | HUNK | HUNK | HUNK | HUNK |
| CMGC | ICK | ICK | ICK | ICK |
| TK | IGF1R | IGF1R | IGF1R | IGF1R |
| Ser/Thr | IKKA | IKKA | IKKA | IKKA |
| Ser/Thr | IKKB | IKKB | IKKB | IKKB |
| Ser/Thr | IKKE | IKKE | IKKE | IKKE |
| TKL | ILK | ILK | ILK | ILK |
| TK | INSR | INSR | INSR | INSR |
| TK | INSRR | INSRR | INSRR | INSRR |
| Metabolic | IPMK | IPMK | IPMK | IPMK |
| TKL | IRAK1 | IRAK1 | IRAK1 | IRAK1 |
| TKL | IRAK3 | IRAK3 | IRAK3 | IRAK3 |
| TKL | IRAK4 | IRAK4 | IRAK4 | IRAK4 |
| TK | JAK1 | JAK1 | JAK1 | JAK1 |
| TK | JAK3 | JAK3 | JAK3 | JAK3 |
| Metabolic | K6PF | K6PF | K6PF | K6PF |
| Metabolic | K6PL | K6PL | K6PL | K6PL |
| Metabolic | K6PP | K6PP | K6PP | K6PP |
| CAMK | KALRN | KALRN | KALRN | KALRN |
| AGC | KAPCA | KAPCA | KAPCA | KAPCA |
| AGC | KAPCB | KAPCB | KAPCB | KAPCB |
| CK1 | KC1A | KC1A | KC1A | KC1A |
| CK1 | KC1AL | KC1AL | KC1AL | KC1AL |
| CK1 | KC1D | KC1D | KC1D | KC1D |
| CK1 | KC1E | KC1E | KC1E | KC1E |
| CK1 | KC1G1 | KC1G1 | KC1G1 | KC1G1 |
| CK1 | KC1G2 | KC1G2 | KC1G2 | KC1G2 |
| CK1 | KC1G3 | KC1G3 | KC1G3 | KC1G3 |
| CAMK | KCC1A | KCC1A | KCC1A | KCC1A |
| CAMK | KCC1G | KCC1G | KCC1G | KCC1G |
| CAMK | KCC2A | KCC2A | KCC2A | KCC2A |
| CAMK | KCC2B | KCC2B | KCC2B | KCC2B |
| CAMK | KCC2D | KCC2D | KCC2D | KCC2D |
| CAMK | KCC2G | KCC2G | KCC2G | KCC2G |
| CAMK | KCC4 | KCC4 | KCC4 | KCC4 |
| AGC | KGP1A | KGP1A | KGP1A | KGP1A |
| AGC | KGP1B | KGP1B | KGP1B | KGP1B |
| AGC | KGP2 | KGP2 | KGP2 | KGP2 |
| TK | KIT | KIT | KIT | KIT |
| Metabolic | KITM | KITM | KITM | KITM |
| AGC | KPCA | KPCA | KPCA | KPCA |
| AGC | KPCB | KPCB | KPCB | KPCB |
| AGC | KPCD | KPCD | KPCD | KPCD |
| CAMK | KPCD1 | KPCD1 | KPCD1 | KPCD1 |
| CAMK | KPCD2 | KPCD2 | KPCD2 | KPCD2 |
| CAMK | KPCD3 | KPCD3 | KPCD3 | KPCD3 |
| AGC | KPCG | KPCG | KPCG | KPCG |
| CAMK | KPSH2 | KPSH2 | KPSH2 | KPSH2 |
| AGC | KS6A1 | KS6A1 | KS6A1 | KS6A1 |
| AGC | KS6A2 | KS6A2 | KS6A2 | KS6A2 |
| AGC | KS6A3 | KS6A3 | KS6A3 | KS6A3 |
| AGC | KS6A4 | KS6A4 | KS6A4 | KS6A4 |
| AGC | KS6A5 | KS6A5 | KS6A5 | KS6A5 |
| AGC | KS6A6 | KS6A6 | KS6A6 | KS6A6 |
| AGC | KS6C1 | KS6C1 | KS6C1 | KS6C1 |
| TKL | KSR2 | KSR2 | KSR2 | KSR2 |
| TK | KSYK | KSYK | KSYK | KSYK |
| Metabolic | KT3K | KT3K | KT3K | KT3K |
| AGC | LATS1 | LATS1 | LATS1 | LATS1 |
| AGC | LATS2 | LATS2 | LATS2 | LATS2 |
| TK | LCK | LCK | LCK | LCK |
| TKL | LIMK1 | LIMK1 | LIMK1 | LIMK1 |
| TKL | LIMK2 | LIMK2 | LIMK2 | LIMK2 |
| TKL | LRRK2 | LRRK2 | LRRK2 | LRRK2 |
| TK | LYN | LYN | LYN | LYN |
| STE | M3K1 | M3K1 | M3K1 | M3K1 |
| STE | M3K11 | M3K11 | M3K11 | M3K11 |
| STE | M3K15 | M3K15 | M3K15 | M3K15 |
| STE | M3K2 | M3K2 | M3K2 | M3K2 |
| STE | M3K3 | M3K3 | M3K3 | M3K3 |
| STE | M3K4 | M3K4 | M3K4 | M3K4 |
| STE | M3K5 | M3K5 | M3K5 | M3K5 |
| STE | M3K6 | M3K6 | M3K6 | M3K6 |
| STE | M3K9 | M3K9 | M3K9 | M3K9 |
| STE | M4K2 | M4K2 | M4K2 | M4K2 |
| STE | M4K3 | M4K3 | M4K3 | M4K3 |
| STE | M4K4 | M4K4 | M4K4 | M4K4 |
| STE | M4K5 | M4K5 | M4K5 | M4K5 |
| CMGC | MAK | MAK | MAK | MAK |
| CAMK | MARK1 | MARK1 | MARK1 | MARK1 |
| CAMK | MARK2 | MARK2 | MARK2 | MARK2 |
| CAMK | MARK3 | MARK3 | MARK3 | MARK3 |
| CAMK | MARK4 | MARK4 | MARK4 | MARK4 |
| AGC | MAST1 | MAST1 | MAST1 | MAST1 |
| AGC | MAST2 | MAST2 | MAST2 | MAST2 |
| AGC | MAST3 | MAST3 | MAST3 | MAST3 |
| AGC | MAST4 | MAST4 | MAST4 | MAST4 |
| TK | MATK | MATK | MATK | MATK |
| CAMK | MELK | MELK | MELK | MELK |
| TK | MERTK | MERTK | MERTK | MERTK |
| TK | MET | MET | MET | MET |
| STE | MINK1 | MINK1 | MINK1 | MINK1 |
| CMGC | MK01 | MK01 | MK01 | MK01 |
| CMGC | MK03 | MK03 | MK03 | MK03 |
| CMGC | MK04 | MK04 | MK04 | MK04 |
| CMGC | MK06 | MK06 | MK06 | MK06 |
| CMGC | MK07 | MK07 | MK07 | MK07 |
| CMGC | MK08 | MK08 | MK08 | MK08 |
| CMGC | MK09 | MK09 | MK09 | MK09 |
| CMGC | MK10 | MK10 | MK10 | MK10 |
| CMGC | MK11 | MK11 | MK11 | MK11 |
| CMGC | MK12 | MK12 | MK12 | MK12 |
| CMGC | MK13 | MK13 | MK13 | MK13 |
| CMGC | MK14 | MK14 | MK14 | MK14 |
| CMGC | MK15 | MK15 | MK15 | MK15 |
| STE | MLTK | MLTK | MLTK | MLTK |
| CMGC | MP2K1 | MP2K1 | MP2K1 | MP2K1 |
| CMGC | MP2K2 | MP2K2 | MP2K2 | MP2K2 |
| CMGC | MP2K3 | MP2K3 | MP2K3 | MP2K3 |
| CMGC | MP2K4 | MP2K4 | MP2K4 | MP2K4 |
| CMGC | MP2K5 | MP2K5 | MP2K5 | MP2K5 |
| CMGC | MP2K6 | MP2K6 | MP2K6 | MP2K6 |
| AGC | MRCKB | MRCKB | MRCKB | MRCKB |
| AGC | MRCKG | MRCKG | MRCKG | MRCKG |
| TK | MUSK | MUSK | MUSK | MUSK |
| CAMK | MYLK | MYLK | MYLK | MYLK |
| CAMK | MYLK2 | MYLK2 | MYLK2 | MYLK2 |
| CAMK | MYLK4 | MYLK4 | MYLK4 | MYLK4 |
| Metabolic | NDKA | NDKA | NDKA | NDKA |
| Metabolic | NDKB | NDKB | NDKB | NDKB |
| Metabolic | NDKM | NDKM | NDKM | NDKM |
| Ser/Thr | NEK1 | NEK1 | NEK1 | NEK1 |
| Ser/Thr | NEK10 | NEK10 | NEK10 | NEK10 |
| Ser/Thr | NEK2 | NEK2 | NEK2 | NEK2 |
| Ser/Thr | NEK3 | NEK3 | NEK3 | NEK3 |
| Ser/Thr | NEK5 | NEK5 | NEK5 | NEK5 |
| Ser/Thr | NEK6 | NEK6 | NEK6 | NEK6 |
| Ser/Thr | NEK7 | NEK7 | NEK7 | NEK7 |
| Ser/Thr | NEK9 | NEK9 | NEK9 | NEK9 |
| CAMK | NIM1 | NIM1 | NIM1 | NIM1 |
| CMGC | NLK | NLK | NLK | NLK |
| TK | NTRK3 | NTRK3 | NTRK3 | NTRK3 |
| CAMK | NUAK1 | NUAK1 | NUAK1 | NUAK1 |
| CAMK | NUAK2 | NUAK2 | NUAK2 | NUAK2 |
| CAMK | OBSCN | OBSCN | OBSCN | OBSCN |
| STE | PAK4 | PAK4 | PAK4 | PAK4 |
| CMGC | PCTK1 | PCTK1 | PCTK1 | PCTK1 |
| CMGC | PCTK2 | PCTK2 | PCTK2 | PCTK2 |
| CMGC | PCTK3 | PCTK3 | PCTK3 | PCTK3 |
| Ser/Thr | PDK1L | PDK1L | PDK1L | PDK1L |
| Metabolic | PDK3 | PDK3 | PDK3 | PDK3 |
| Metabolic | PDXK | PDXK | PDXK | PDXK |
| CMGC | PFTK1 | PFTK1 | PFTK1 | PFTK1 |
| TK | PGFRA | PGFRA | PGFRA | PGFRA |
| TK | PGFRB | PGFRB | PGFRB | PGFRB |
| CAMK | PHKG2 | PHKG2 | PHKG2 | PHKG2 |
| Ser/Thr | PI3R4 | PI3R4 | PI3R4 | PI3R4 |
| Metabolic | PI42A | PI42A | PI42A | PI42A |
| Metabolic | PI42B | PI42B | PI42B | PI42B |
| Metabolic | PI42C | PI42C | PI42C | PI42C |
| Metabolic | PK3C3 | PK3C3 | PK3C3 | PK3C3 |
| AGC | PKN2 | PKN2 | PKN2 | PKN2 |
| AGC | PKN3 | PKN3 | PKN3 | PKN3 |
| Ser/Thr | PLK1 | PLK1 | PLK1 | PLK1 |
| Ser/Thr | PLK4 | PLK4 | PLK4 | PLK4 |
| Ser/Thr | PMYT1 | PMYT1 | PMYT1 | PMYT1 |
| Atypical | PRKDC | PRKDC | PRKDC | PRKDC |
| Ser/Thr | PRPK | PRPK | PRPK | PRPK |
| TK | PTK6 | PTK6 | PTK6 | PTK6 |
| TK | PTK7 | PTK7 | PTK7 | PTK7 |
| CAMK | QSK | QSK | QSK | QSK |
| TKL | RAF1 | RAF1 | RAF1 | RAF1 |
| Atypical | RIOK2 | RIOK2 | RIOK2 | RIOK2 |
| TKL | RIPK1 | RIPK1 | RIPK1 | RIPK1 |
| TKL | RIPK2 | RIPK2 | RIPK2 | RIPK2 |
| TKL | RIPK3 | RIPK3 | RIPK3 | RIPK3 |
| TK | RON | RON | RON | RON |
| TK | ROS | ROS | ROS | ROS |
| Ser/Thr | SBK1 | SBK1 | SBK1 | SBK1 |
| CAMK | SIK1 | SIK1 | SIK1 | SIK1 |
| CAMK | SIK2 | SIK2 | SIK2 | SIK2 |
| STE | SLK | SLK | SLK | SLK |
| CAMK | SPEG | SPEG | SPEG | SPEG |
| TK | SRC | SRC | SRC | SRC |
| TK | SRMS | SRMS | SRMS | SRMS |
| CMGC | SRPK1 | SRPK1 | SRPK1 | SRPK1 |
| CMGC | SRPK2 | SRPK2 | SRPK2 | SRPK2 |
| CMGC | SRPK3 | SRPK3 | SRPK3 | SRPK3 |
| CAMK | ST17B | ST17B | ST17B | ST17B |
| STE | STK10 | STK10 | STK10 | STK10 |
| CAMK | STK11 | STK11 | STK11 | STK11 |
| Ser/Thr | STK16 | STK16 | STK16 | STK16 |
| STE | STK24 | STK24 | STK24 | STK24 |
| STE | STK25 | STK25 | STK25 | STK25 |
| STE | STK3 | STK3 | STK3 | STK3 |
| Ser/Thr | STK31 | STK31 | STK31 | STK31 |
| Ser/Thr | STK35 | STK35 | STK35 | STK35 |
| Ser/Thr | STK36 | STK36 | STK36 | STK36 |
| STE | STK4 | STK4 | STK4 | STK4 |
| CAMK | STK40 | STK40 | STK40 | STK40 |
| AGC | STK6 | STK6 | STK6 | STK6 |
| STE | STRAA | STRAA | STRAA | STRAA |
| STE | TAOK1 | TAOK1 | TAOK1 | TAOK1 |
| STE | TAOK2 | TAOK2 | TAOK2 | TAOK2 |
| STE | TAOK3 | TAOK3 | TAOK3 | TAOK3 |
| Ser/Thr | TBK1 | TBK1 | TBK1 | TBK1 |
| TK | TEC | TEC | TEC | TEC |
| TKL | TESK1 | TESK1 | TESK1 | TESK1 |
| Ser/Thr | TEX14 | TEX14 | TEX14 | TEX14 |
| TKL | TGFR1 | TGFR1 | TGFR1 | TGFR1 |
| TKL | TGFR2 | TGFR2 | TGFR2 | TGFR2 |
| TK | TIE1 | TIE1 | TIE1 | TIE1 |
| TK | TIE2 | TIE2 | TIE2 | TIE2 |
| CAMK | TITIN | TITIN | TITIN | TITIN |
| Ser/Thr | TLK1 | TLK1 | TLK1 | TLK1 |
| Ser/Thr | TLK2 | TLK2 | TLK2 | TLK2 |
| STE | TNIK | TNIK | TNIK | TNIK |
| TK | TNK1 | TNK1 | TNK1 | TNK1 |
| Atypical | TRPM6 | TRPM6 | TRPM6 | TRPM6 |
| Ser/Thr | TTK | TTK | TTK | TTK |
| TK | TYK2 | TYK2 | TYK2 | TYK2 |
| TK | TYRO3 | TYRO3 | TYRO3 | TYRO3 |
| Ser/Thr | ULK3 | ULK3 | ULK3 | ULK3 |
| Ser/Thr | ULK4 | ULK4 | ULK4 | ULK4 |
| TK | VGFR1 | VGFR1 | VGFR1 | VGFR1 |
| TK | VGFR2 | VGFR2 | VGFR2 | VGFR2 |
| TK | VGFR3 | VGFR3 | VGFR3 | VGFR3 |
| CK1 | VRK2 | VRK2 | VRK2 | VRK2 |
| Ser/Thr | WEE1 | WEE1 | WEE1 | WEE1 |
| Ser/Thr | WNK1 | WNK1 | WNK1 | WNK1 |
| TK | YES | YES | YES | YES |
| TK | ZAP70 | ZAP70 | ZAP70 | ZAP70 |

Table 3 shows the kinases and their families found in the sample from the TNBC patient, C3Tag mouse, SUM159 and MDA cell lines. The kinase names in bold were detected in the sample of interest. A total of 320 kinases were detected. For comparison, the R&D Systems RTK Array only has 40 kinases (AXL, c-Ret, Dtk, EGFR, EphA1, EphA2, EphA3, EphA4, EphA6, EphA7, EphB1, EphB2, EphB4, EphB6, FGFR1, FGFR2, FGFR3, FGFR4, flt3, HER2, HER3, HER4, HGF R, IGF1-R, INSR, M-CSF R, Mer, MSP R, MuSK, PDGFRa, PDGFRb, ROR1, ROR2, SCF R, Tie-1, Tie-2, TrkA, TrKB, VEGFR1, VEGFR2, VEGFR3).

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A multi-analyte column comprising a first and a second layer wherein:
(a) the first layer comprises a first solid support having at least two different affinity ligands with specific kinase binding affinity; and
(b) the second layer comprises a second solid support having at least two different affinity ligands with non-specific kinase binding affinity.

2. The multi-analyte column of claim 1, wherein the first solid support has specific binding affinity for one or more tyrosine kinases.

3. The multi-analyte column of claim 1, wherein the first solid support has specific binding affinity for one or more serine/threonine kinases.

4. The multi-analyte column of claim 1, wherein the specific binding affinities are for kinases selected from the group consisting of Abl, ATK, BRAF, c-KIT, COT, EGFR, FLT-3, HER1, HER2, HER3, HER4, IGF-1R, InsR, LYN, MEK, MET, P38, PDGFRβ, PKC/GSK3β, Src, and VEGFR.

5. The multi-analyte column of claim 4, wherein the specific binding affinities are for kinases selected from the group consisting of Abl, EGFR, HER2, LYN, P38, and PKC/GSK3β.

6. The multi-analyte column of claim 1, wherein each affinity ligand of the first solid support binds 20 or fewer kinases.

7. The multi-analyte column of claim 6, wherein the affinity ligands are selected from the group consisting of a bisindoylmaleimide-X ligand, a GW-572016 ligand, and a SB203580 ligand.

8. The multi-analyte column of claim 1, wherein each affinity ligand of the second solid support binds 50 or more kinases.

9. The multi-analyte column of claim 8, wherein the affinity ligands are selected from the group consisting of a 2,4-diaminopyrimidine, pyrazole ligand, PP58 ligand, purvalanol B ligand, and a VI16832 ligand.

10. The multi-analyte column of claim 1 wherein the specific binding affinity kinase solid supports and the non-specific binding kinase solid supports are present in a molar ratio of ranging from about 4:1 to about 1:4.

11. The multi-analyte column of claim 10 wherein the specific binding affinity kinase solid supports and the non-specific binding kinase solid supports are present in a molar ratio of ranging from about 1.5:1 to about 1:1.5.

12. The multi-analyte column of claim 1, wherein the first solid support comprises at least three different affinity ligands having specific kinase binding affinity.

13. The multi-analyte column of claim 1, wherein the second solid support comprises at least three different affinity ligands having non-specific kinase binding affinity.

14. The multi-analyte column of claim 1, wherein the first solid support comprises at least three different affinity ligands having specific kinase binding affinity and the second solid support comprises at least three different affinity ligands having non-specific kinase binding affinity.

15. A method for detecting low abundant kinases in a sample comprising:
(a) loading a sample on a multi-analyte column comprising a first and a second layer wherein:
(i) the first layer comprises a first solid support having at least two different affinity ligands with specific kinase binding affinity; and
(ii) the second layer comprises a second solid support having at least two different affinity ligands with non-specific kinase binding affinity;
(b) washing the multi-analyte column to remove any unbound proteins;
(c) eluting any kinases bound to the multi-analyte column with a denaturing agent; and
(d) detecting the eluted kinases.

16. The method of claim 15 wherein the detection in step (d) is done by mass spectrometry.

17. The method of claim 16 wherein the method is performed on a plurality of samples and at least one sample is labeled with a detectable label.

18. The method of claim 17 wherein the detectable label is prepared by SILAC (stable isotope labeling with amino acids in cell culture).

19. The method of claim 16, wherein an isotope labeled spike is added to the sample.

20. The method of claim 15, wherein greater than 150 kinases are detected from a 5 mg protein portion of the sample.

21. The method of claim 20, wherein greater than 180 kinases are detected from the 5 mg protein portion of the sample.

22. The method of claim 15, wherein 40 or more kinases are detected from a single sample and changes in phosphorylation states of the kinases are also measured.

23-42. (canceled)
